(19) United States
(12) Patent Application Publication
(10) Pub. No.: US 2010/0035857 A1
(43) Pub. Date: Feb. 11, 2010

(54) ANTI-HYPERCHOLESTEROLEMIC COMPOUNDS

(76) Inventors: Robert J. DeVita, Westfield, NJ (US); Gregori J. Morriello, Randolph, NJ (US); Peter Lin, Edison, NJ (US)

Correspondence Address: MERCK AND CO., INC, P O BOX 2000, RAHWAY, NJ, 07065-0907, US

(21) Appl. No.: 12/518,771

(22) PCT Filed: Dec. 14, 2007
(86) PCT No.: PCT/US2007/025638

§ 371 Date: Jun. 11, 2009

Related U.S. Application Data
(60) Provisional application No. 60/875,889, filed on Dec. 20, 2006.

(51) Int. Cl. A61K 31/397; C07D 205/08; A61P 7/00

(52) U.S. Cl. 514/210.02; 540/200

(57) ABSTRACT

This invention provides cholesterol absorption inhibitors of Formula I:I and the pharmaceutically acceptable salts thereof. The compounds are useful for lowering plasma cholesterol levels, particularly LDL cholesterol, and for treating and preventing atherosclerosis and atherosclerotic disease events.

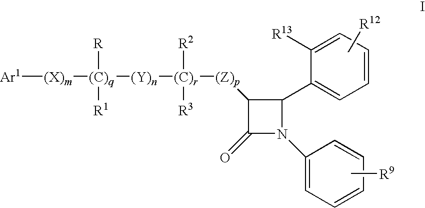

### BACKGROUND OF THE INVENTION

The instant invention relates to substituted 2-azetidinones and the pharmaceutically acceptable salts and esters there of, and to their use alone or in combination with other active agents to treat hypercholesterolemia and for preventing, halting or slowing the progression of atherosclerosis and related conditions and disease events.

It has been clear for several decades that elevated blood cholesterol is a major risk factor for coronary heart disease, and many studies have shown that the risk of CHD events can be reduced by lipid-lowering therapy. Prior to 1987, the lipid-lowering armamentarium was limited essentially to a low saturated fat and cholesterol diet, the bile acid sequestrants (cholestyramine and colestipol), nicotinic acid (niacin), the fibrates and probucol. Unfortunately, all of these treatments have limited efficacy or tolerability, or both. Substantial reductions in LDL (low density lipoprotein) cholesterol accompanied by increases in HDL (high density lipoprotein) cholesterol could be achieved by the combination of a lipid-lowering diet and a bile acid sequestrant, with or without the addition of nicotinic acid. However, this therapy is not easy to administer or tolerate and was therefore often unsuccessful except in specialist lipid clinics. The fibrates produce a moderate reduction in LDL cholesterol accompanied by increased HDL cholesterol and a substantial reduction in triglycerides, and because they are well tolerated these drugs have been more widely used. Probucol produces only a small reduction in LDL cholesterol and also reduces HDL cholesterol, which, because of the strong inverse relationship between HDL cholesterol level and CHD risk, is generally considered undesirable. With the introduction of lovastatin, the first inhibitor of HMG-CoA reductase to become available for prescription in 1987, for the first time physicians were able to obtain large reductions in plasma cholesterol with very few adverse effects.

Recent studies have unequivocally demonstrated that lovastatin, simvastatin and pravastatin, all members of the HMG-CoA reductase inhibitor class, slow the progression of atherosclerotic lesions in the coronary and carotid arteries. Simvastatin and pravastatin have also been shown to reduce the risk of coronary heart disease events, and in the case of simvastatin a highly significant reduction in the risk of coronary death and total mortality has been shown by the Scandinavian Simvastatin Survival Study. This study also provided some evidence for a reduction in cerebrovascular events. Despite the substantial reduction in the risk of coronary morbidity and mortality achieved by simvastatin, the risk is still substantial in the treated patients. For example, in the Scandinavian Simvastatin Survival Study, the 42% reduction in the risk of coronary death still left 5% of the treated patients to die of their disease over the course of this 5 year study. Further reduction of risk is clearly needed.

A more recent class of anti-hyperlipidemic agents that has emerged includes inhibitors of cholesterol absorption. Ezetimibe, the first compound to receive regulatory approval in this class, is currently marketed in the U.S. under the tradename ZETIA®. Ezetimibe has the following chemical structure and is described in U.S. Pat. No.'s Re. 37721 and 5,846,966:

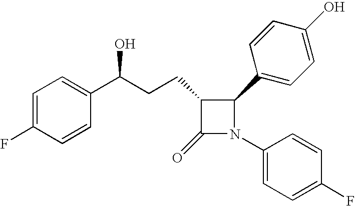

Sugar-substituted 2-azetidinones, including glucuronidated analogs of the following general structure:

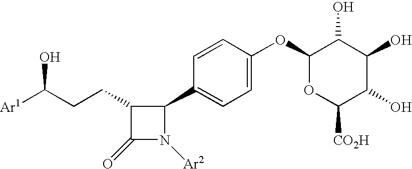

and methods for making them are disclosed in U.S. Pat. No. 5,756,470, wherein Ar¹ and Ar² are unsubstituted or substituted aryl groups.

Additional cholesterol absorption inhibitors are described in WO2002/066464 A1 (applied for by Kotobuki Pharmaceutical Co.), and US2002/0137689 A1 (Glombik et al.). WO2002/066464 A1 discloses hypolipidemic compounds of general formula

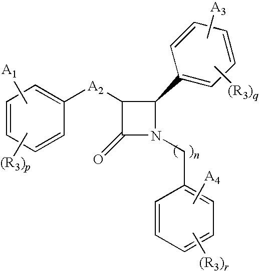

wherein, among other definitions, A₁, A₃ and A₄ can be

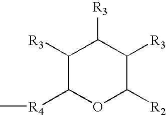

and wherein R₂ is —CH₂OH, —CH₂OC(O)—R₁, or —CO₂R₁; R₃ is —OH or —OC(O)R₁, and R₄ is —(CH₂)ₖR₅(CH₂)ᵢ— where k and i are zero or integers of one or more, and k+i is an integer of 10 or less; and R₅ is a single bond, —CH═CH—, —OCH₂—, carbonyl or —CH(OH).

US2002/0137689 A1 discloses hypolipidemic compounds of general formula

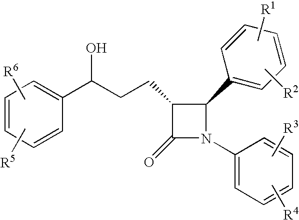

wherein, among other definitions, R¹, R², R³, R⁴, R⁵, R⁶ independently of one another can be (C₀-C₃₀)-alkylene-(LAG), where one or more carbon atoms of the alkylene radical may be replaced by —O—, —(C═O)—, —CH═CH—, —C≡C—, —N((C₁-C₆)-alkyl)-, —N((C₁-C₆)-alkylphenyl) or —NH—; and (LAG) is a sugar residue, disugar residue, trisugar residue, tetrasugar residue; a sugar acid, or an amino sugar.

In the ongoing effort to discover novel treatments for hyperlipidemia and atherosclerotic process, the instant invention provides novel cholesterol absorption inhibitors, described below.

### SUMMARY OF THE INVENTION

One object of the instant invention is to provide novel cholesterol absorption inhibitors of Formula I

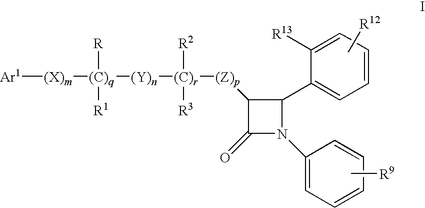

and the pharmaceutically acceptable salts thereof.

A second object of the instant invention is to provide a method for inhibiting cholesterol absorption comprising administering a therapeutically effective amount of a compound of Formula I to a patient in need of such treatment. Another object is to provide a method for reducing plasma cholesterol levels, especially LDL-cholesterol, and treating hypercholesterolemia comprising administering a therapeutically effective amount of a compound of Formula I to a patient in need of such treatment.

As a further object, methods are provided for preventing or reducing the risk of developing atherosclerosis, as well as for halting or slowing the progression of atherosclerotic disease once it has become clinically evident, comprising the administration of a prophylactically or therapeutically effective amount, as appropriate, of a compound of Formula I to a patient who is at risk of developing atherosclerosis or who already has atherosclerotic disease. Another object of the present invention is the use of the compounds of the present invention for the manufacture of a medicament useful in treating, preventing or reducing the risk of developing these conditions. Other objects of this invention are to provide processes for making the compounds of Formula I and to provide novel pharmaceutical compositions comprising these compounds.

Additionally the compounds of this invention, particularly radioactive isotopes of the compounds of Formula I, can be used in screening assays, where the assay is designed to identify new cholesterol absorption inhibitors that have the same mechanism of action as ezetimibe. Additional objects will be evident from the following detailed description.

### DETAILED DESCRIPTION OF THE INVENTION

The novel cholesterol absorption inhibitors of the instant invention include compounds of structural Formula I

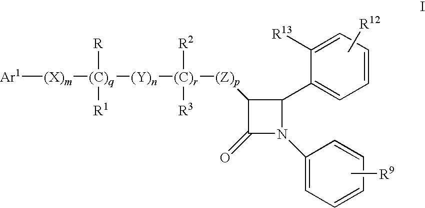

and the pharmaceutically acceptable salts thereof, wherein
Ar¹ is selected from the group consisting of aryl and R⁴-substituted aryl;
X, Y and Z are independently selected from the group consisting of —CH₂—, —CH(C₁₋₆alkyl)- and —C(C₁₋₆alkyl)₂-;
R is selected from the group consisting of —OR⁶, —O(CO)R⁶, —O(CO)OR⁸, —O(CO)NR⁶R⁷, a sugar residue, a disugar residue, a trisugar residue and a tetrasugar residue;
R¹ is selected from the group consisting of —H, —C₁₋₆alkyl and aryl, or R and R¹ together are oxo;
R² is selected from the group consisting of —OR⁶, —O(CO)R⁶, —O(CO)OR⁸ and —O(CO)NR⁶R⁷;
R³ is selected from the group consisting of —H, —C₁₋₆alkyl and aryl, or R² and R³ together are oxo;
q and r are integers each independently selected from 0 and 1 provided that at least one of q and r is 1;
m, n and p are integers each independently selected from 0, 1, 2, 3 and 4, provided that the sum of m, n, p, q and r is 1, 2, 3, 4, 5 or 6;
t is an integer selected from 0, 1 and 2;
R⁴ is 1-5 substituents independently selected at each occurrence from the group consisting of: —OR⁵, —O(CO)R⁵, —O(CO)OR⁸, —O—C₁₋₅alkyl-OR⁵, —O(CO)NR⁵R⁶, —NR⁵R⁶, —NR⁵(CO)R⁶, —NR⁵(CO)OR⁸, —NR⁵(CO)NR⁶R⁷, —NR⁵SO₂R⁸, —COOR⁵, —CONR⁵R⁶, —COR⁵, —SO₂NR⁵R⁶, —S(O)ₜR⁸, —O—C₁₋₁₀ alkyl-COOR⁵, —O—C₁₋₁₀ alkyl-CONR⁵R⁶ and fluoro;
R⁵, R⁶ and R⁷ are independently selected at each occurrence from the group consisting of —H, —C₁₋₆alkyl, aryl and aryl-substituted-C₁₋₆alkyl;
R⁸ is selected from the group consisting of —C₁₋₆alkyl, aryl and aryl-substituted-C₁₋₆alkyl;
R⁹ is selected from the group consisting of chloro, fluoro,
   —C≡C—C₁₋₆alkyl-NR¹⁰R¹¹,
   —(CH₂)ₓCH═CH—C₁₋₆alkyl-NR¹⁰R¹¹,
   —C₁₋₈alkyl-NR¹⁰R¹¹,
   —C≡C—C₁₋₄alkyl-CH—(CH₂—NR¹⁰R¹¹)₂,
   —(CH₂)ₓCH═CH—C₁₋₄alkyl —CH—(CH₂—NR¹⁰R¹¹)₂,
   —C₁₋₆alkyl-CH—(CH₂—NR¹⁰R¹¹)₂
   —C≡C—C₁₋₆alkyl-R^{11a},
   —(CH₂)ₓCH═CH—C₁₋₆alkyl-R^{11a},
   —C₁₋₈ alkyl-R^{11a},
   —C≡C—C₁₋₆alkyl,
   —(CH₂)ₓCH═CH—C₁₋₆alkyl,
   —C₁₋₈alkyl,
   —C₂₋₁₅alkynyl mono- or poly-substituted with —OH and optionally substituted with R¹⁴,
   —C₂₋₁₅alkenyl mono- or poly-substituted with —OH and optionally substituted with R¹⁴,
   —C₁₋₁₅alkyl mono- or poly-substituted with —OH and optionally substituted with R¹⁴, and
x is an integer selected from 0, 1 and 2;
R¹⁰ is independently selected at each occurrence from the group consisting of —H and —C₁₋₃alkyl;
R¹¹ is independently selected at each occurrence from the group consisting of —H, —C₁₋₃alkyl, —C(O)—C₁₋₃alkyl, —C(O)—NR¹⁰R¹⁰, —SO₂—C₁₋₃alkyl and —SO₂-phenyl;
R^{11a} is selected from the group consisting of —C(O)—NR¹⁰R¹⁰, —SO₂—C₁₋₃alkyl, and —SO₂-phenyl;
R¹² is selected from the group consisting of —C₂₋₁₅alkynyl mono- or poly-substituted with —OH and optionally substituted with R¹⁴, —C₂₋₁₅alkenyl mono- or poly-substituted with —OH and optionally substituted with R¹⁴, —C₁₋₁₅alkyl mono- or poly-substituted with —OH and optionally substituted with R¹⁴;
R¹³ is selected from the group consisting of —H and —OH; and
R¹⁴ is a sugar residue optionally substituted with —COOH, —COOC₁₋₃alkyl and —C₁₋₃alkyl-OH;
provided that when R⁹ is selected from the group consisting of —C≡C—(CH₂)₁₋₆—NR¹⁰R¹¹, —CH═CH—(CH₂)₁₋₆—NR¹⁰R¹¹ and —(CH₂)₁₋₈—NR¹⁰R¹¹, then R¹² is not selected from the group consisting of —C₁₋₁₅alkyl mono- or poly-substituted with —OH, —CH═CH—C₁₋₁₃alkyl mono- or poly-substituted with —OH, —C≡C—C₁₋₁₃alkyl mono- or poly-substituted with —OH,

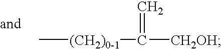

and excluding (3R,4S)-4-{4-[3,4-dihydroxy-3-(hydroxymethyl)butyl]phenyl}-3-[(3S)-3-(4-fluorophenyl)-3-hydroxypropyl]-1-[4-(3-hydroxypropyl)phenyl]azetidin-2-one.

In an embodiment of this invention are compounds of Formula I wherein the sum of m, q and n is 1, 2, 3, 4, or 5 when p is O and r is 1.

In another embodiment of this invention are compounds of Formula I wherein r is zero and m is zero, i.e., compounds of structural Formula Ic:

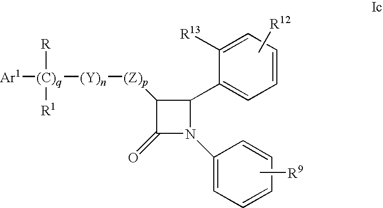

and the pharmaceutically acceptable salts thereof, wherein the variables (Ar¹, R, R¹, R⁹, R¹², R¹³, Y, Z, q, n, p) are as defined in Formula I.

In another embodiment of this invention are compounds Formula I having structural Formula Ia,

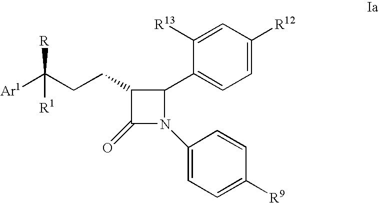

and the pharmaceutically acceptable salts thereof, wherein the variables (Ar¹, R, R¹, R⁹, R¹², R¹³) are as defined in Formula I.

In another embodiment of this invention are compounds Formula I having structural Formula Ib,

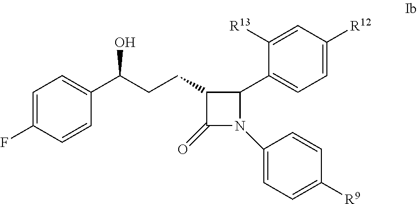

and the pharmaceutically acceptable salts thereof, wherein the variables (R⁹, R¹², R¹³) are as defined in Formula I.

In another embodiment of this invention are compounds of Formula I or Ia wherein Ar¹ is selected from the group consisting of aryl and R⁴-substituted aryl wherein R⁴ is 1-2 substituents independently selected at each occurrence from the group consisting of: —OR⁵, —O(CO)R⁵, —O(CO)OR⁸, —O—C₁₋₅alkyl-OR⁵, —O(CO)NR⁵R⁶, —NR⁵R⁶, —NR⁵(CO)R⁶, —NR⁵(CO)OR⁸, —NR⁵(CO)NR⁶R⁷, —NR⁵SO₂R⁸, —COOR⁵, —CONR⁵R⁶, —COR⁵, —SO₂NR⁵R⁶, —S(O)ₜR⁸, —O—C₁₋₁₀alkyl-COOR⁵, —O—C₁₋₁₀alkyl-CONR⁵R⁶ and fluoro. In a class of this embodiment, Ar¹ is unsubstituted, mono- or di-substituted phenyl. In a sub-class, Ar¹ is phenyl mono-substituted with fluoro, and particularly 4-fluoro-phenyl.

In another embodiment of this invention are compounds of Formula I or Ia wherein R is —OR⁶; in a class of this embodiment, R is —OH.

In another embodiment of this invention are compounds of Formula I or Ia wherein R¹ is —H.

In another embodiment of this invention are compounds of Formula I or Ia wherein R² is —OR⁶; in a class of this embodiment, R² is —OH.

In another embodiment of this invention are compounds of Formula I or Ia wherein R³ is —H.

In another embodiment of this invention are compounds of Formula I, Ia or Ib wherein R⁹ is selected from the group consisting of —C≡C—C₁₋₆alkyl-NR¹⁰R¹¹, —(CH₂)ₓCH═CH—C₁₋₄alkyl-NR¹⁰R¹¹, —C₁₋₈alkyl-NR¹⁰R¹¹, —C≡C≡C₁₋₄alkyl-CH—(CH₂—NR¹⁰R¹¹)₂, —(CH₂)ₓCH═CH—C₁₋₄alkyl-CH—(CH₂—NR¹⁰R¹¹)₂, and —C₁₋₆alkyl-CH—(CH₂—NR¹⁰R¹¹)₂. More particularly, R⁹ is selected from —C₁₋₈alkyl-NR¹⁰R¹¹ and —C₁₋₆alkyl-CH—(CH₂—NR¹⁰R¹¹)₂. In a class of this embodiment, R¹¹ is selected from —SO₂—C₁₋₃alkyl and —SO₂-phenyl, and more particularly it is —SO₂CH₃. In another class of this embodiment, R¹² is —C₁₋₁₅alkyl mono- or poly-substituted with —OH and optionally substituted with R¹⁴. In another class of this embodiment, R¹² is —C₃₋₆alkyl substituted with one to three of —OH and substituted with R¹⁴.

In another embodiment of this invention are compounds of Formula I, Ia or Ib wherein R⁹ is selected from the group consisting of —C₂₋₁₅alkynyl mono- or poly-substituted with —OH and optionally substituted with R¹⁴, —C₂₋₁₅alkenyl mono- or poly-substituted with —OH and optionally substituted with R¹⁴, and —C₁₋₁₅alkyl mono- or poly-substituted with —OH and optionally substituted with R¹⁴. More particularly, R⁹ is selected from the group consisting of —C₁₋₈alkyl mono- or poly-substituted with —OH and optionally substituted with R¹⁴, —(CH₂)ₓCH═CH—C₁₋₆alkyl mono- or poly-substituted with —OH and optionally substituted with R¹⁴, and —C≡C—C₁₋₆alkyl mono- or poly-substituted with —OH and optionally substituted with R¹⁴. In a class of this embodiment, R¹² is —C₃₋₆alkyl mono-, di-, or tri-substituted with —OH and optionally substituted with R¹⁴.

In another embodiment of this invention are compounds of Formula I, Ia or Ib wherein R⁹ is selected from the group consisting of —C≡C—C₁₋₆alkyl-R^{11a}, —(CH₂)ₓCH═CH—C₁₋₁₄alkyl-R^{11a} and —C₁₋₈ alkyl-R^{11a}. In a class of this embodiment, R¹² is —C₃₋₆alkyl mono-, di-, or tri-substituted with —OH and optionally substituted with R¹⁴.

In another embodiment of this invention are compounds of Formula I, Ia or Ib wherein R¹² is selected from the group consisting of —C₂₋₁₅alkynyl mono- or poly-substituted with —OH and substituted with R¹⁴, —C₂₋₁₅alkenyl mono- or poly-substituted with —OH and substituted with R¹⁴, and —C₁₋₁₅alkyl mono- or poly-substituted with —OH and substituted with R¹⁴. In a class of this embodiment, R¹² is selected from the group consisting of —C₁₋₈alkyl mono- or poly-substituted with —OH and optionally substituted with R¹⁴, —(CH₂)ₓCH═CH—C₁₋₆alkyl mono- or poly-substituted with —OH and optionally substituted with R¹⁴, and —C≡C—C₁₋₆alkyl mono- or poly-substituted with —OH and optionally substituted with R¹⁴. In a sub-class of this class, R¹² is —C₁₋₈alkyl substituted with 1, 2, 3, 4, or 5 of —OH and optionally substituted with R¹⁴.

In another embodiment of this invention are compounds of Formula I, Ia or Ib wherein R¹² is selected from the group consisting of —C₂₋₁₅alkynyl mono- or poly-substituted with —OH, —C₂₋₁₅alkenyl mono- or poly-substituted with —OH, and —C₁₋₁₅alkyl mono- or poly-substituted with —OH. In a class of this embodiment, R¹² is selected from the group consisting of —C₁₋₈alkyl mono- or poly-substituted with —OH and optionally substituted with R¹⁴, —(CH₂)ₓCH═CH—C₁₋₆alkyl mono- or poly-substituted with —OH and optionally substituted with R¹⁴, and —C≡C—C₁₋₆alkyl mono- or poly-substituted with —OH and optionally substituted with R¹⁴. In a sub-class of this class, R¹² is —C₁₋₈alkyl substituted with 1, 2, 3, 4, or 5 of —OH and optionally substituted with R¹⁴.

In another embodiment of this invention are compounds of Formula I, Ia or Ib wherein R¹³ is —H.

In another embodiment of this invention are compounds of Formula I, Ia, or Ib wherein R¹⁴ is selected from the group consisting of

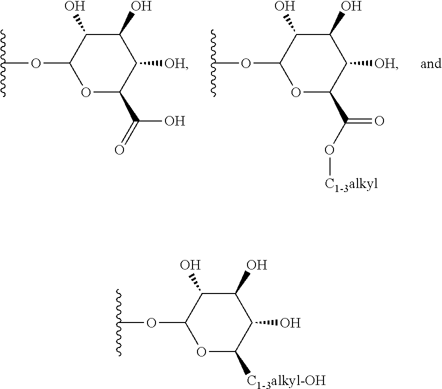

In another embodiment of this invention are compounds of Formula I, Ia or Ic further excluding (3R,4S)-4-{4-[3,4-dihydroxy-3-(hydroxymethyl)butyl]phenyl}-3-[(3S)-3-(4-fluorophenyl)-3-oxopropyl]-1-[4-(3-hydroxypropyl)phenyl]azetidin-2-one.

Compounds within the scope of Formulas I, Ia, Ib and Ic include but are not limited to:

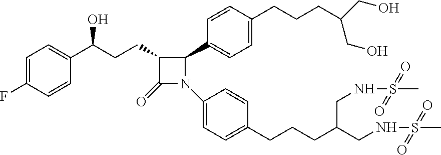

1) N-(5-[4-((2S,3R)-3-[(3S)-3-(4-fluorophenyl)-3-hydroxypropyl]-2-{4-[5-hydroxy-4-(hydroxymethyl)pentyl]phenyl}-4-oxoazetidin-1-yl)phenyl]-2-{[(methylsulfonyl)amino]methyl}pentyl)methanesulfonamide;

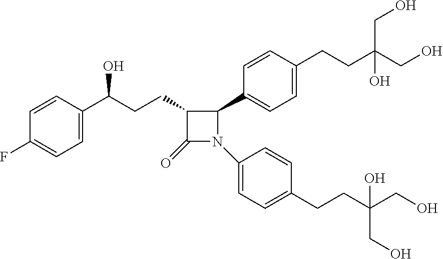

2) (3R,4S)-1,4-bis{4-[3,4-dihydroxy-3-(hydroxymethyl)butyl]phenyl}-3-[(3S)-3-(4-fluorophenyl)-3-hydroxypropyl]azetidin-2-one;

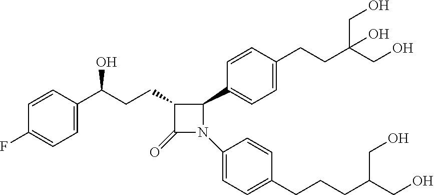

3) (3R,4S)-4-{4-[3,4-dihydroxy-3-(hydroxymethyl)butyl]phenyl}-3-[(3S)-3-(4-fluorophenyl)-3-hydroxypropyl]-1-{4-[5-hydroxy-4-(hydroxymethyl)pentyl]-phenyl}azetidin-2-one;

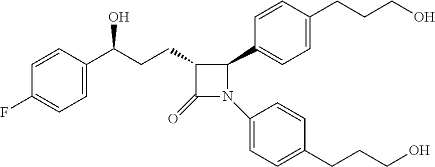

4) (3R,4S)-3-[(3S)-3-(4-fluorophenyl)-3-hydroxypropyl]-1,4-bis[4-(3-hydroxypropyl)phenyl]azetidin-2-one;

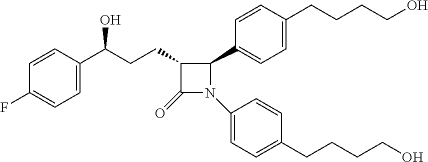

5) (3R,4S)-3-[(3S)−)-3-(4-fluorophenyl)-3-hydroxypropyl]-1,4-bis[4-(4-hydroxybutyl)phenyl]azetidin-2-one;

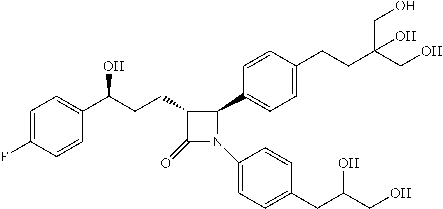

6) (3R,4S)-4-{4-[3,4-dihydroxy-3-(hydroxymethyl)butyl]phenyl}-1-[4-(2,3-dihydroxypropyl)phenyl]-3-[(3S—)-3-(4-fluorophenyl)-3-hydroxypropyl]azetidin-2-one;

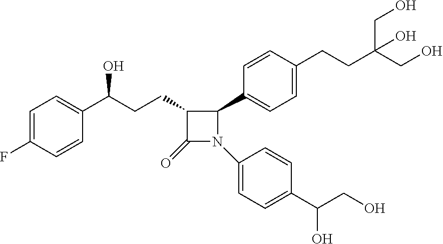

7) (3R,4S)-1-[4-(1,2-dihydroxyethyl)phenyl]-4-{4-[3,4-dihydroxy-3-(hydroxymethyl)butyl]phenyl}-3-[(3S)-3-(4-fluorophenyl)-3-hydroxypropyl]azetidin-2-one;

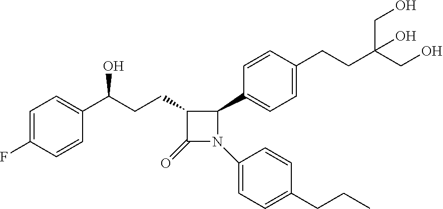

8) (3R,4S)-4-{4-[3,4-dihydroxy-3-(hydroxymethyl)butyl]phenyl}-3-[(3S)-3-(4-fluorophenyl)-3-hydroxypropyl]-1-(4-propylphenyl)azetidin-2-one;

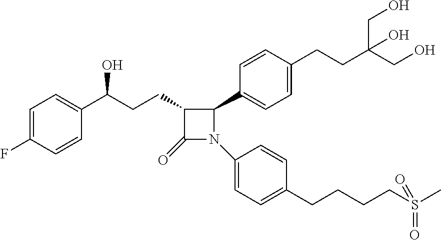

9) (3R,4S)-4-{4-[3,4-dihydroxy-3-(hydroxymethyl)butyl]phenyl}-3-[(3S)-3-(4-fluorophenyl)-3-hydroxypropyl]-1-{4-[4-(methylsulfonyl)butyl]phenyl}azetidin-2-one;

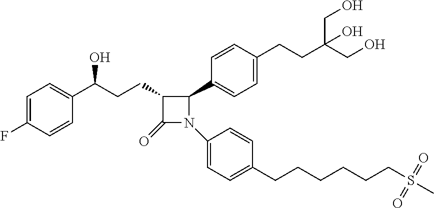

10) (3R,4S)-4-{4-[3,4-dihydroxy-3-(hydroxymethyl)butyl]phenyl}-3-[(3S)-3-(4-fluorophenyl)-3-hydroxypropyl]-1-{4-[6-(methylsulfonyl)hexyl]phenyl}azetidin-2-one;

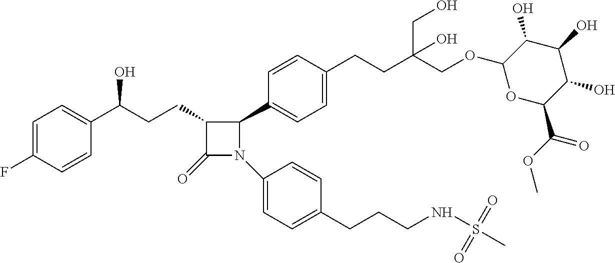

11) methyl (2S,3S,4S,5R)-6-[4-{4-[(2S,3R)-3-[(3S)-3-(4-fluorophenyl)-3-hydroxypropyl]-1-(4-{3-[(methylsulfonyl)amino]propyl}phenyl)-4-oxoazetidin-2-yl]phenyl}-2-hydroxy-2-(hydroxymethyl)butoxy]-3,4,5-trihydroxytetrahydro-2H-pyran-2-carboxylate; and

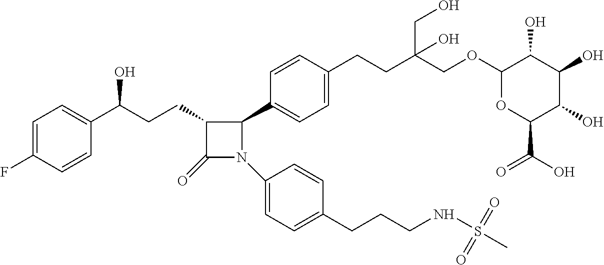

12) (2S,3S,4S,5R)-6-[4-{4-[(2S,3R)-3-[(3S)-3-(4-fluorophenyl)-3-hydroxypropyl]-1-(4-{3-[(methylsulfonyl)amino]propyl}phenyl)-4-oxoazetidin-2-yl]phenyl}-2-hydroxy-2-(hydroxymethyl)butoxy]-3,4,5-trihydroxytetrahydro-2H-pyran-2-carboxylic acid;
   and pharmaceutically acceptable salts thereof.

Another aspect of this invention includes the following cholesterol absorption inhibitor compounds:

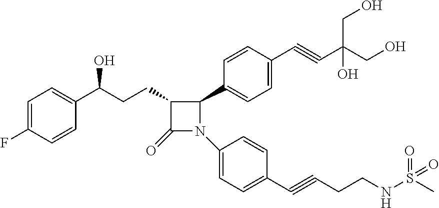

1) N-[4-(4-{(2S,3R)-2-{4-[3,4-dihydroxy-3-(hydroxymethyl)but-1-yn-1-yl]phenyl}-3-[(3S)-3-(4-fluorophenyl)-3-hydroxypropyl]-4-oxoazetidin-1-yl}phenyl)but-3-yn-1-yl]methanesulfonamide;

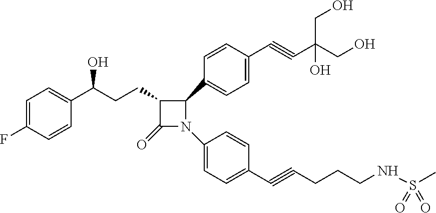

2) N-[5-(4-{(2S,3R)-2-{4-[3,4-dihydroxy-3-(hydroxymethyl)but-1-yn-1-yl]phenyl}-3-[(3S)-3-(4-fluorophenyl)-3-hydroxypropyl]-4-oxoazetidin-1-yl}phenyl)pent-4-yn-1-yl]methanesulfonamide;

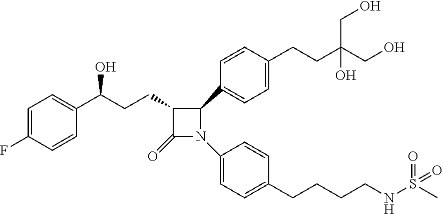

3) N-[4-(4-{(2S,3R)-2-{4-[3,4-dihydroxy-3-(hydroxymethyl)butyl]phenyl}-3-[(3S)-3-(4-fluorophenyl)-3-hydroxypropyl]-4-oxoazetidin-1-yl}phenyl)butyl]methanesulfonamide;

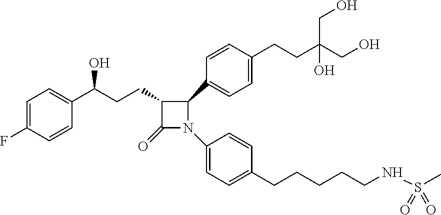

4) N-[5-(4-{(2S,3R)-2-{4-[3,4-dihydroxy-3-(hydroxymethyl)butyl]phenyl}-3-[(3S)-3-(4-fluorophenyl)-3-hydroxypropyl]-4-oxoazetidin-1-yl}phenyl)pentyl]methanesulfonamide;

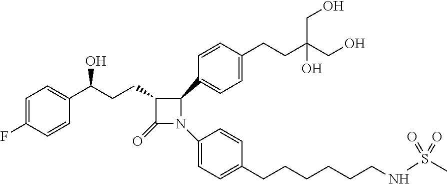

5) N-[6-(4-{(2S,3R)-2-{4-[3,4-dihydroxy-3-(hydroxymethyl)butyl]phenyl}-3-[(3S)-3-(4-fluorophenyl)-3-hydroxypropyl]-4-oxoazetidin-1-yl}phenyl)hexyl]methanesulfonamide;

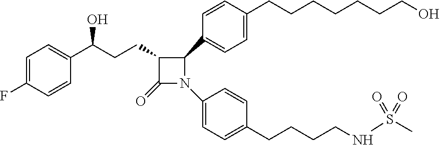

6) N-[4-(4-{(2S,3R)-3-[(3S)-3-(4-fluorophenyl)-3-hydroxypropyl]-2-[4-(7-hydroxyheptyl)phenyl]-4-oxoazetidin-1-yl}phenyl)butyl]methanesulfonamide;

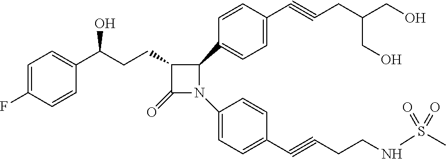

7) N-[4-(4-((2S,3R)-3-[(3S)-3-(4-fluorophenyl)-3-hydroxypropyl]-2-{4-[5-hydroxy-4-(hydroxymethyl)pent-1-yn-1-yl]phenyl}-4-oxoazetidin-1-yl)phenyl]but-3-yn-1-yl}methanesulfonamide;

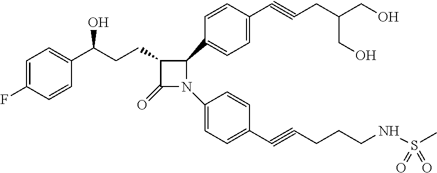

8) N-[5-(4-((2S,3R)-3-[(3S)-3-(4-fluorophenyl)-3-hydroxypropyl]-2-{4-[5-hydroxy-4-(hydroxymethyl)pent-1-yn-1-yl]phenyl}-4-oxoazetidin-1-yl)phenyl]pent-4-yn-1-yl}methanesulfonamide;

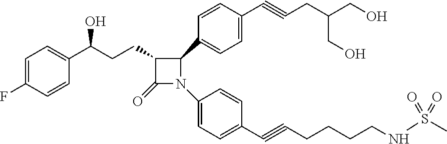

9) N-{6-[4-((2S,3R)-3-[(3S)-3-(4-fluorophenyl)-3-hydroxypropyl]-2-{4-[5-hydroxy-4-(hydroxymethyl)pent-1-yn-1-yl]phenyl}-4-oxoazetidin-1-yl)phenyl]hex-5-yn-1-yl}methanesulfonamide;

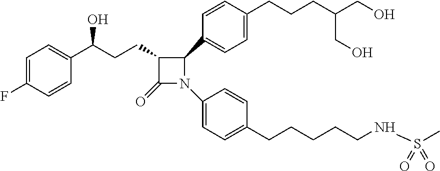

10) N-{5-[4-((2S,3R)-3-[(3S)-3-(4-fluorophenyl)-3-hydroxypropyl]-2-{4-[5-hydroxy-4-(hydroxymethyl)pentyl]phenyl}-4-oxoazetidin-1-yl)phenyl]pentyl}methanesulfonamide;

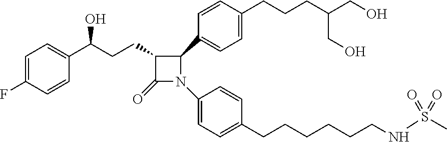

11) N-{6-[4-((2S,3R)-3-[(3S)-3-(4-fluorophenyl)-3-hydroxypropyl]-2-{4-[5-hydroxy-4-(hydroxymethyl)pentyl]phenyl}-4-oxoazetidin-1-yl)phenyl]hexyl}methanesulfonamide;

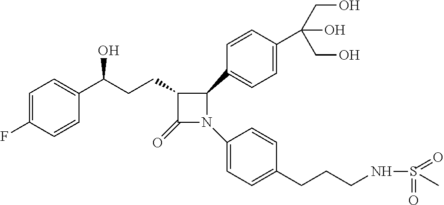

12) N-[3-(4-{(2S,3R)-2-{4-[1,2-dihydroxy-1-(hydroxymethyl)ethyl]phenyl}-3-[(3S)-3-(4-fluorophenyl)-3-hydroxypropyl]-4-oxoazetidin-1-yl}phenyl)propyl]methanesulfonamide;

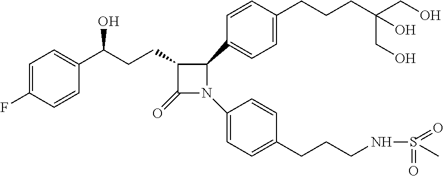

13) N-[3-(4-{(2S,3R)-2-{4-[4,5-dihydroxy-4-(hydroxymethyl)pentyl]phenyl}-3-[(3S)-3-(4-fluorophenyl)-3-hydroxypropyl]-4-oxoazetidin-1-yl}phenyl)propyl]methanesulfonamide;

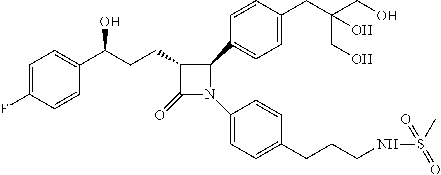

14) N-[3-(4-{(2S,3R)-2-{4-[2,3-dihydroxy-2-(hydroxymethyl)propyl]phenyl}-3-[(3S)-3-(4-fluorophenyl)-3-hydroxypropyl]-4-oxoazetidin-1-yl}phenyl)propyl]methanesulfonamide;

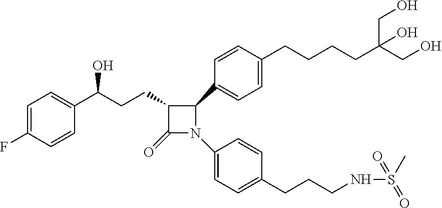

15) N-[3-(4-{(2S,3R)-2-{4-[5,6-dihydroxy-5-(hydroxymethyl)hexyl]phenyl}-3-[(3S)-3-(4-fluorophenyl)-3-hydroxypropyl]-4-oxoazetidin-1-yl}phenyl)propyl]methanesuslfonamide; and

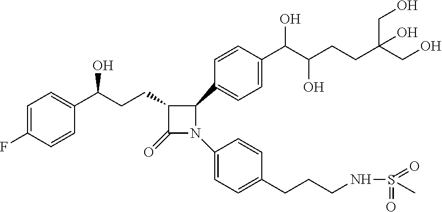

16) N-{3-[4-((3R,4S)-3-[(3S)-3-(4-fluorophenyl)-3-hydroxypropyl]-2-oxo-4-{4-[1,2,5,6-tetrahydroxy-5-(hydroxymethyl)hexyl]phenyl}azetidin-1-yl)phenyl]propyl}methanesulfonamide;
   and pharmaceutically acceptable salts thereof.

Also presented are the following compounds: N-[3-(4-{(2S,3R)-2-{4-[3,4-dihydroxy-3-(hydroxymethyl)butyl]phenyl}-3-[3-(4-fluorophenyl)-3-oxopropyl]-4-oxoazetidin-1-yl}phenyl)propyl]methanesulfonamide; (2S,3S,4S,5R,6R)-6-{[(1S)-3-[(2S,3R)-2-{4-[3,4-dihydroxy-3-(hydroxymethyl)butyl]phenyl}-1-(4-{3-[(methylsulfonyl)amino]propyl}phenyl)-4-oxoazetidin-3-yl]-1-(4-fluorophenyl)propyl]oxy}-3,4,5-trihydroxytetrahydro-2H-pyran-2-carboxylic acid; and (3R,4S)-4-{4-[3,4-dihydroxy-3-(hydroxymethyl)butyl]phenyl}-3-[(3S)-3-(4-fluorophenyl)-3-hydroxypropyl]-1-[4-(3-hydroxypropyl)phenyl]azetidin-2-one, and the pharmaceutically acceptable salts thereof; as well as a pharmaceutical composition comprised of one of said compounds with a pharmaceutically acceptable carrier.

Each embodiment, class or sub-class described above for each variable (i.e., Ar¹, R, R¹, R⁹, R¹², etc.) in Formulas I, Ia and Ib may be combined with one or more of the embodiments, classes or sub-classes described above for one or more other variables, and all such sub-generic combinations are included within the scope of this invention. For example, one or more embodiments, classes or sub-classes described above for the variables can be combined with the Formula Ic embodiment (i.e, Formula I compounds wherein r is zero and m is zero). A further example is a sub-genus composed of compounds of Formula Ia wherein Ar¹ is unsubstituted, mono- or di-substituted phenyl, R is —OR⁶ and R¹² is —C₁₋₈alkyl substituted with 1, 2, 3, 4, or 5 of —OH and optionally substituted with R¹⁴. All such sub-generic combinations are encompassed in the scope of this invention and are not limited to these examples.

As used herein “alkyl” is intended to include both branched- and straight-chain saturated aliphatic hydrocarbon groups having the specified number of carbon atoms. Examples of alkyl groups include, but are not limited to, methyl (Me), ethyl (Et), n-propyl (Pr), n-butyl (Bu), n-pentyl, n-hexyl, and the isomers thereof such as isopropyl (i-Pr), isobutyl (i-Bu), secbutyl (s-Bu), tertbutyl (t-Bu), 1-methylpropyl, 2-methylbutyl, 3-methylbutyl, isopentyl, isohexyl and the like.

Certain alkyl, alkenyl and alkynyl groups defined herein may be “mono- or poly-substituted with —OH,” meaning that one or more hydroxyl substituents is present on the alkyl group, and that each carbon atom available for substitution in the alkyl group may independently be unsubstituted or mono-substituted with hydroxyl provided that at least one carbon atom is substituted with hydroxyl. This encompasses alkyl groups where every available carbon atom is mono-substituted with hydroxyl as well as those where fewer than all available carbon atoms are mono-substituted with hydroxyl.

As used herein, “aryl” is intended to include phenyl (Ph), naphthyl, indenyl, tetrahydronaphthyl or indanyl. Phenyl is preferred.

Hydroxyl protecting groups may be used on intermediates during the synthetic procedures for making final products within the scope of this invention. Suitable protecting groups (designated as “PG” herein) for the hydroxyl groups, for example those in R¹² and R¹³, include but are not limited to those that are known to be useful as hydroxyl protecting groups, such as for example benzyl, acetyl, benzoyl, tert-butyldiphenylsilyl, trimethylsilyl, para-methoxybenzyl, benzylidine, dimethylacetal and methoxy methyl. Conditions required to selectively add and remove such protecting groups are found in standard textbooks such as Greene, T, and Wuts, P. G. M., Protective Groups in Organic Synthesis, John Wiley & Sons, Inc., New York, N.Y., 1999.

Compounds of Formula I may contain one or more asymmetric centers and can thus occur as racemates and racemic mixtures, single enantiomers, enantiomeric mixtures, diastereomeric mixtures and individual diastereomers. All such isomeric forms of the compounds of Formula I are included within the scope of this invention. Furthermore, some of the crystalline forms for compounds of the present invention may exist as polymorphs and as such are intended to be included in the present invention. In addition, some of the compounds of the instant invention may form solvates with water or organic solvents. Such hydrates and solvates are also encompassed within the scope of this invention. Some of the compounds described herein contain olefinic double bonds. The invention includes both E and Z geometric isomers.

Due to their activity as cholesterol absorption inhibitors, the compounds of the present invention can be used in screening assays, where the assay is designed to identify new cholesterol absorption inhibitors. Radioactive isotopes of the compounds of Formula I are particularly useful in such assays, for example compounds of Formula I wherein sulfur is replaced with “hot” —³⁵S—, and particularly wherein the radioactive sulfur isotope is incorporated within the R⁹ moiety. All such radioactive isotopes of the compounds of Formula I are included within the scope of this invention.

Reference to the compounds of this invention as those of “Formula I,” “Formula Ia,” and “Formula Ib” is intended herein to encompass compounds falling within the scope of each of these structural formulas including pharmaceutically acceptable salts and esters thereof where such salts and esters are possible. Herein, the term “pharmaceutically acceptable salts” means non-toxic salts of the compounds employed in this invention which are generally prepared by reacting the free acid with a suitable organic or inorganic base, particularly those formed from cations such as sodium, potassium, aluminum, calcium, lithium, magnesium, zinc and tetramethylammonium, as well as those salts formed from amines such as ammonia, ethylenediamine, N-methylglucamine, lysine, arginine, ornithine, choline, N,N′-dibenzylethylenediamine, chloroprocaine, diethanolamine, procaine, N-benzylphenethylamine, 1-p-chlorobenzyl-2-pyrrolidine-1′-yl-methylbenzimidazole, diethylamine, piperazine, morpholine, 2,4,4-trimethyl-2-pentamine and tris(hydroxymethyl)aminomethane.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

Also, in the case of a carboxylic acid (—COOH) or alcohol group being present in the compounds of this invention, pharmaceutically acceptable esters of carboxylic acid derivatives, such as —C₁₋₄ alkyl, —C₁₋₄ alkyl substituted with phenyl, acetylamino and pivaloyloxymethyl, or acyl derivatives of alcohols, such as O-acetyl, O-pivaloyl, O-benzoyl, O-dimethylamino and O-aminoacyl, can be employed. Included within the scope of this invention are those esters and acyl groups known in the art for modifying the solubility or hydrolysis characteristics of a compound for use as a pro-drug or for sustained-release formulation.

The term “patient” includes mammals, especially humans, who use the instant active agents for the prevention or treatment of a medical condition. Administering of the drug to the patient includes both self-administration and administration to the patient by another person. The patient may be in need of treatment for an existing disease or medical condition, or may desire prophylactic treatment to prevent or reduce the risk for diseases and medical conditions affected by inhibition of cholesterol absorption.

The term “therapeutically effective amount” is intended to mean that amount of a pharmaceutical drug that will elicit the biological or medical response of a tissue, a system, animal or human that is being sought by a researcher, veterinarian, medical doctor or other clinician. The term “prophylactically effective amount” is intended to mean that amount of a pharmaceutical drug that will prevent or reduce the risk of occurrence of the biological or medical event that is sought to be prevented in a tissue, a system, animal or human by a researcher, veterinarian, medical doctor or other clinician. Particularly, the dosage a patient receives can be selected so as to achieve the amount of LDL cholesterol lowering desired; the dosage a patient receives may also be titrated over time in order to reach a target LDL level. The dosage regimen utilizing a compound of the instant invention is selected in accordance with a variety of factors including type, species, age, weight, sex and medical condition of the patient; the severity of the condition to be treated; the potency of the compound chosen to be administered; the route of administration; and the renal and hepatic function of the patient. A consideration of these factors is well within the purview of the ordinarily skilled clinician for the purpose of determining the therapeutically effective or prophylactically effective dosage amount needed to prevent, counter, or arrest the progress of the condition.

The compounds of the instant invention are cholesterol absorption inhibitors and are useful for reducing plasma cholesterol levels, particularly reducing plasma LDL cholesterol levels, when used either alone or in combination with another active agent, such as an anti-atherosclerotic agent, and more particularly a cholesterol biosynthesis inhibitor, for example an HMG-CoA reductase inhibitor. Thus the instant invention provides methods for inhibiting cholesterol absorption and for treating lipid disorders including hypercholesterolemia, comprising administering a therapeutically effective amount of a compound of Formula I to a person in need of such treatment. Further provided are methods for preventing or reducing the risk of developing atherosclerosis, as well as for halting or slowing the progression of atherosclerotic disease once it has become clinically evident, comprising the administration of a prophylactically or therapeutically effective amount, as appropriate, of a compound of Formula I to a mammal who is at risk of developing atherosclerosis or who already has atherosclerotic disease.

Atherosclerosis encompasses vascular diseases and conditions that are recognized and understood by physicians practicing in the relevant fields of medicine. Atherosclerotic cardiovascular disease including restenosis following revascularization procedures, coronary heart disease (also known as coronary artery disease or ischemic heart disease), cerebrovascular disease including multi-infarct dementia, and peripheral vessel disease including erectile dysfunction are all clinical manifestations of atherosclerosis and are therefore encompassed by the terms “atherosclerosis” and “atherosclerotic disease.”

A compound of Formula I may be administered to prevent or reduce the risk of occurrence, or recurrence where the potential exists, of a coronary heart disease event, a cerebrovascular event, and/or intermittent claudication. Coronary heart disease events are intended to include CHD death, myocardial infarction (i.e., a heart attack), and coronary revascularization procedures. Cerebrovascular events are intended to include ischemic or hemorrhagic stroke (also known as cerebrovascular accidents) and transient ischemic attacks. Intermittent claudication is a clinical manifestation of peripheral vessel disease. The term “atherosclerotic disease event” as used herein is intended to encompass coronary heart disease events, cerebrovascular events, and intermittent claudication. It is intended that persons who have previously experienced one or more non-fatal atherosclerotic disease events are those for whom the potential for recurrence of such an event exists.

Accordingly, the instant invention also provides a method for preventing or reducing the risk of a first or subsequent occurrence of an atherosclerotic disease event comprising the administration of a prophylactically effective amount of a compound of Formula I to a patient at risk for such an event. The patient may or may not have atherosclerotic disease at the time of administration, or may be at risk for developing it.

Persons to be treated with the instant therapy include those at risk of developing atherosclerotic disease and of having an atherosclerotic disease event. Standard atherosclerotic disease risk factors are known to the average physician practicing in the relevant fields of medicine. Such known risk factors include but are not limited to hypertension, smoking, diabetes, low levels of high density lipoprotein (HDL) cholesterol, and a family history of atherosclerotic cardiovascular disease. Published guidelines for determining those who are at risk of developing atherosclerotic disease can be found in: Executive Summary of the Third Report of the National Cholesterol Education Program (NCEP) Expert Panel on Detection, Evaluation, and Treatment of High Blood Cholesterol in Adults (Adult Treatment Panel III), JAMA, 2001; 285 pp. 2486-2497. People who are identified as having one or more of the above-noted risk factors are intended to be included in the group of people considered at risk for developing atherosclerotic disease. People identified as having one or more of the above-noted risk factors, as well as people who already have atherosclerosis, are intended to be included within the group of people considered to be at risk for having an atherosclerotic disease event.

The oral dosage amount of the compound of Formula I is from about 0.1 to about 30 mg/kg of body weight per day, preferably about 0.1 to about 15 mg/kg of body weight per day. For an average body weight of 70 kg, the dosage level is therefore from about 5 mg to about 1000 mg of drug per day. However, dosage amounts will vary depending on factors as noted above, including the potency of the particular compound. Although the active drug of the present invention may be administered in divided doses, for example from two to four times daily, a single daily dose of the active drug is preferred. As examples, the daily dosage amount may be selected from, but not limited to, 5 mg, 10 mg, 15 mg, 20 mg, 25 mg, 40 mg, 50 mg, 75 mg, 80 mg, 100 mg and 200 mg.

The active drug employed in the instant therapy can be administered in such oral forms as tablets, capsules, pills, powders, granules, elixirs, tinctures, suspensions, syrups, and emulsions. Oral formulations are preferred, and particularly solid oral formulations such as tablets.

For compounds of Formula I, administration of the active drug can be via any pharmaceutically acceptable route and in any pharmaceutically acceptable dosage form. This includes the use of oral conventional rapid-release, time controlled-release and delayed-release (such enteric coated) pharmaceutical dosage forms. Additional suitable pharmaceutical compositions for use with the present invention are known to those of ordinary skill in the pharmaceutical arts; for example, see Remington's Pharmaceutical Sciences, Mack Publishing Co., Easton, Pa.

In the methods of the present invention, the active drug is typically administered in admixture with suitable pharmaceutical diluents, excipients or carriers (collectively referred to herein as “carrier” materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with a non-toxic, pharmaceutically acceptable, inert carrier such as lactose, starch, sucrose, glucose, modified sugars, modified starches, methyl cellulose and its derivatives, dicalcium phosphate, calcium sulfate, mannitol, sorbitol and other reducing and non-reducing sugars, magnesium stearate, steric acid, sodium stearyl fumarate, glyceryl behenate, calcium stearate and the like. For oral administration in liquid form, the drug components can be combined with non-toxic, pharmaceutically acceptable inert carrier such as ethanol, glycerol, water and the like. Moreover, when desired or necessary, suitable binders, lubricants, disintegrating agents and coloring and flavoring agents can also be incorporated into the mixture. Stabilizing agents such as antioxidants, for example butylated hydroxyanisole (BHA), 2,6-di-tert-butyl-4-methylphenol (BHT), propyl gallate, sodium ascorbate, citric acid, calcium metabisulphite, hydroquinone, and 7-hydroxycoumarin, particularly BHA, propyl gallate and combinations thereof, can also be added to stabilize the dosage forms. When a compound of Formula I is formulated together with an HMG-CoA reductase inhibitor such as simvastatin, the use of at least one stabilizing agent is preferred in the composition. Other suitable components include gelatin, sweeteners, natural and synthetic gums such as acacia, tragacanth or alginates, carboxymethylcellulose, polyethylene glycol, waxes and the like.

The instant invention also encompasses a process for preparing a pharmaceutical composition comprising combining a compound of Formula I with a pharmaceutically acceptable carrier. Also encompassed is the pharmaceutical composition which is made by combining a compound of Formula I with a pharmaceutically acceptable carrier.

One or more additional active agents may be administered in combination with a compound of Formula I, and therefore an embodiment of the instant invention encompasses a drug combination. The drug combination encompasses a single dosage formulation comprised of the compound of Formula I and additional active agent or agents, as well as administration of each of the compound of Formula I and the additional active agent or agents in separate dosage formulations, which allows for concurrent or sequential administration of the active agents. The additional active agent or agents can be lipid modifying agents, particularly a cholesterol biosynthesis inhibitor such as an HMG-CoA reductase inhibitor, or agents having other pharmaceutical activities, or agents that have both lipid-modifying effects and other pharmaceutical activities. Examples of HMG-CoA reductase inhibitors useful for this purpose include statins in their lactonized or dihydroxy open acid forms and pharmaceutically acceptable salts and esters thereof, including but not limited to lovastatin (MEVACOR®; see U.S. Pat. No. 4,342,767); simvastatin (ZOCOR®; see U.S. Pat. No. 4,444,784); dihydroxy open-acid simvastatin, particularly the ammonium or calcium salts thereof; pravastatin, particularly the sodium salt thereof (PRAVACOL®; see U.S. Pat. No. 4,346,227); fluvastatin particularly the sodium salt thereof (LESCOL®; see U.S. Pat. No. 5,354,772); atorvastatin, particularly the calcium salt thereof (LIPITOR®; see U.S. Pat. No. 5,273,995); rosuvastatin (CRESTOR®; see U.S. Pat. No. 5,260,440); and pitavastatin also referred to as NK-104 (see PCT international publication number WO 97/23200). Examples of additional active agents which may be employed include but are not limited to one or more of FLAP inhibitors; 5-lipoxygenase inhibitors; additional cholesterol absorption inhibitors such as ezetimibe (ZETIA®), described in U.S. Pat. Nos. Re. 37721 and 5,846,966; cholesterol ester transfer protein (CETP) inhibitors, for example JTT-705 and torcetrapib, also known as CP529,414; HMG-CoA synthase inhibitors; squalene epoxidase inhibitors; squalene synthetase inhibitors (also known as squalene synthase inhibitors); acyl-coenzyme A: cholesterol acyltransferase (ACAT) inhibitors including selective inhibitors of ACAT-1 or ACAT-2 as well as dual inhibitors of ACAT1 and -2; microsomal triglyceride transfer protein (MTP) inhibitors; niacin; niacin receptor agonists such as acipimox and acifran, as well as niacin receptor partial agonists; LDL (low density lipoprotein) receptor inducers; platelet aggregation inhibitors, for example glycoprotein IIb/IIIa fibrinogen receptor antagonists and aspirin; human peroxisome proliferator activated receptor gamma (PPARγ) agonists including the compounds commonly referred to as glitazones for example pioglitazone and rosiglitazone and, including those compounds included within the structural class known as thiazolidinediones as well as those PPARγ agonists outside the thiazolidinedione structural class; PPARα agonists such as clofibrate, fenofibrate including micronized fenofibrate, and gemfibrozil; PPAR dual α/γ agonists; vitamin B₆ (also known as pyridoxine) and the pharmaceutically acceptable salts thereof such as the HCl salt; vitamin B₁₂ (also known as cyanocobalamin); folic acid or a pharmaceutically acceptable salt or ester thereof such as the sodium salt and the methylglucamine salt; anti-oxidant vitamins such as vitamin C and E and beta carotene; beta-blockers; angiotensin II antagonists such as losartan; angiotensin converting enzyme inhibitors such as enalapril and captopril; calcium channel blockers such as nifedipine and diltiazam; endothelian antagonists; agents that enhance ABC1 gene expression; FXR ligands including both inhibitors and agonists; and LXR ligands including both inhibitors and agonists of all sub-types of this receptor, e.g. LXRα and LXRβ; bisphosphonate compounds such as alendronate sodium; and cyclooxygenase-2 inhibitors such as etoricoxib and celecoxib.

A therapeutically or prophylactically effective amount, as appropriate, of a compound of Formula I can be used for the preparation of a medicament useful for inhibiting cholesterol absorption, as well as for treating and/or reducing the risk for diseases and conditions affected by inhibition of cholesterol absorption, such as treating lipid disorders, preventing or reducing the risk of developing atherosclerotic disease, halting or slowing the progression of atherosclerotic disease once it has become clinically manifest, and preventing or reducing the risk of a first or subsequent occurrence of an atherosclerotic disease event. For example, the medicament may be comprised of about 5 mg to about 1000 mg of a compound of Formula I. The medicament comprised of a compound of Formula I may also be prepared with one or more additional active agents, such as those described supra.

Compounds can be tested for cholesterol absorption activity in assays using, e.g., rats or mice, and are preferably tested in the rat assay described herein. Representative compounds of this invention were determined to inhibit cholesterol absorption employing the Cholesterol Absorption Assay in Rats, below. This assay involves comparing a test compound to ezetimibe with respect to their ability to inhibit cholesterol absorption in rats. Both ezetimibe and the tested compounds of this invention inhibited cholesterol absorption by >75% at the highest dose tested. Preferred compounds inhibited cholesterol absorption by >90%. The tested compounds had an ID 50<10 mg/kg. Preferred compounds had an ID 50<1 mg/kg.

Cholesterol Absorption Assay in Rats: CD male rats (n=5/group), aged 5 weeks, were dosed orally with 0.5 ml 0.25% methyl cellulose solution with or without test compound or ezetimibe (0.0003 to 1 mg/kg). 0.5 to 16 hrs later all of the rats were dosed orally with 0.5 ml INTRALIPID® containing 5 μCi [³H]-cholesterol per rat. Five hours later, the animals were euthanized, and liver and blood were collected. Cholesterol counts in liver and plasma were determined, and percent inhibition of cholesterol absorption was calculated.

Compounds can also be tested for cholesterol absorption activity using a mouse assay, described as follows. Cholesterol Absorption Assay in Mice: C57BL/6 male mice (n=6/group), aged 10-14 weeks, are dosed orally with 0.2 ml 0.25% methyl cellulose solution with or without test compound or ezetimibe (0.12-10 mg/kg). Thirty minutes later all of the mice are dosed orally with 0.2 ml INTRALIPID™ containing 2 μCi [³H]-cholesterol per mouse. Five hours later, the animals were euthanized, and liver and blood are collected. Cholesterol counts in liver and plasma are determined, and percent inhibition of cholesterol absorption is calculated.

The compounds of structural Formula I of the present invention can be prepared according to the procedures of the following Scheme and Examples, using appropriate materials, and are further exemplified by specific examples which follow. Moreover, by utilizing the procedures described herein, one of ordinary skill in the art can readily prepare additional compounds of the present invention claimed herein. The compounds illustrated in the examples are not, however, to be construed as forming the only genus that is considered as the invention. The Examples further illustrate details for the preparation of the compounds of the present invention. Those skilled in the art will readily understand that known variations of the conditions and processes of the following preparative procedures can be used to prepare these compounds.

A variety of chromatographic techniques may be employed in the preparation of the compounds. These techniques include, but are not limited to: High Performance Liquid Chromatography (HPLC) including normal-reversed- and chiral-phase; Medium Pressure Liquid Chromatography (MPLC), Super Critical Fluid Chromatography; preparative Thin Layer Chromatography (prep TLC); flash chromatography with silica gel or reversed-phase silica gel; ion-exchange chromatography; and radial chromatography. All temperatures are degrees Celsius unless otherwise noted.

Some abbreviations used herein include:

Ac Acyl (CH₃C(O)—)

Aq. Aqueous

Bn Benzyl

C. Celsius

calc. Calculated

DCM dichloromethane

DIEA N,N-diisopropylethylamine

DMAP 4-dimethylaminopyridine

DMF N,N-dimethylformamide

equiv. Equivalent(s)

ES-MS Electron Spray Ion-Mass Spectroscopy

EtOAc Ethyl acetate

h Hour(s)

HPLC High performance liquid chromatography

lc-ms Liquid Chromatography-mass spectrometry

min Minute(s)

mp Melting point

MPLC Medium pressure liquid chromatography

MS Mass spectrum

Prep. Preparative

RT (or r.t. or rt) Room temperature

sat. Saturated

TBAI tetrabutylammonium iodide

TBS Tert-butyl dimethylsilyl

TEA Triethyl amine

TFA Trifluoroacetic acid

THF Tetrahydrofuran

TMEDA N,N,N′,N′-Tetramethylethylenediamine

TLC (or tlc) Thin layer chromatography

The general Schemes below illustrate a method for the syntheses of compounds of the present invention. All substituents and variables (e.g., R¹, R², Ar¹, v, w, etc.) are as defined above in Formula I unless indicated otherwise.

In Scheme I, I-1 is treated with a terminal alkyne of type I-2 in the presence of a suitable palladium catalyst such as tetrakistriphenylphosphine palladium(0) or [1,1′-bis(diphenylphosphino)ferrocene]dichloropalladium(II) or the like, and copper(I) iodide. The reaction is usually performed in an inert organic solvent such as DMF, between room temperature and 100° C., for a period of 6-48 h, and the product is an internal alkyne of structural formula I-3. Alkyne I-2 may contain a radioactive atom such as ³⁵S to provide the corresponding radiolabeled adduct upon reaction with I-1. Conversion of I-3 to -I-4 can be achieved by hydrogenation of the triple bond in the R⁹ position, followed by treatment with guanidine and triethylamine in methanol to selectively remove the phenolic acetate; then converting the phenol to the triflate I-4 via treatment with bis(trifluoromethylsulfonyl)amino pyridine in the presence of either triethylamine or N,N diisopropyl-N-ethyl amine in dichloromethane medium. Incorporation of the alkynyl-R^{12a} group is achieved by palladium assisted coupling of the triflate I-4 with either hydroxyl-protected or unprotected alkynyl-R^{12a} derivative I-5. Examples of hydroxyl protecting groups (PG) include, for example, benzyl, acetate, acetal or any other suitable oxygen protecting group, or combinations thereof, compatible with earlier or subsequent chemical reactions. As an example, R^{12a} includes but is not limited to —C₁₋₆alkyl-OBn and

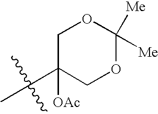

In this method, I-4 is treated with an alkynyl-R^{12a} of type I-5 in the presence of a suitable palladium catalyst such as tetrakistriphenylphosphine palladium(0) and copper(I) iodide with an initiator such as tetrabutylammonium iodide. The reaction is usually performed in an inert organic solvent such as DMF, at 50° C., for a period of 1 to 5 hrs, and the product possesses an alkynyl-R^{12a} of structure I-6. Hydrogenation of the triple bond occurs along with the removal of any benzyl protecting groups contained in R^{12a} by treatment with 10% palladium on carbon catalyst under hydrogen atmosphere in a solvent such as ethyl acetate reacting over 15-24 hours to form I-7. Hydrolysis or cleavage of any remaining hydroxyl protecting groups may be performed at this time, or non-benzylic protecting groups can be removed prior to the hydrogenation step. For example, diols protected as acetals that are contained in R^{12a} may be removed by treatment with aqueous acid. When R^{12a} contains one or more acetate groups, deprotection with potassium cyanide in methanol heated to 50° C. for 1-2 hours affords the free hydroxyl groups.

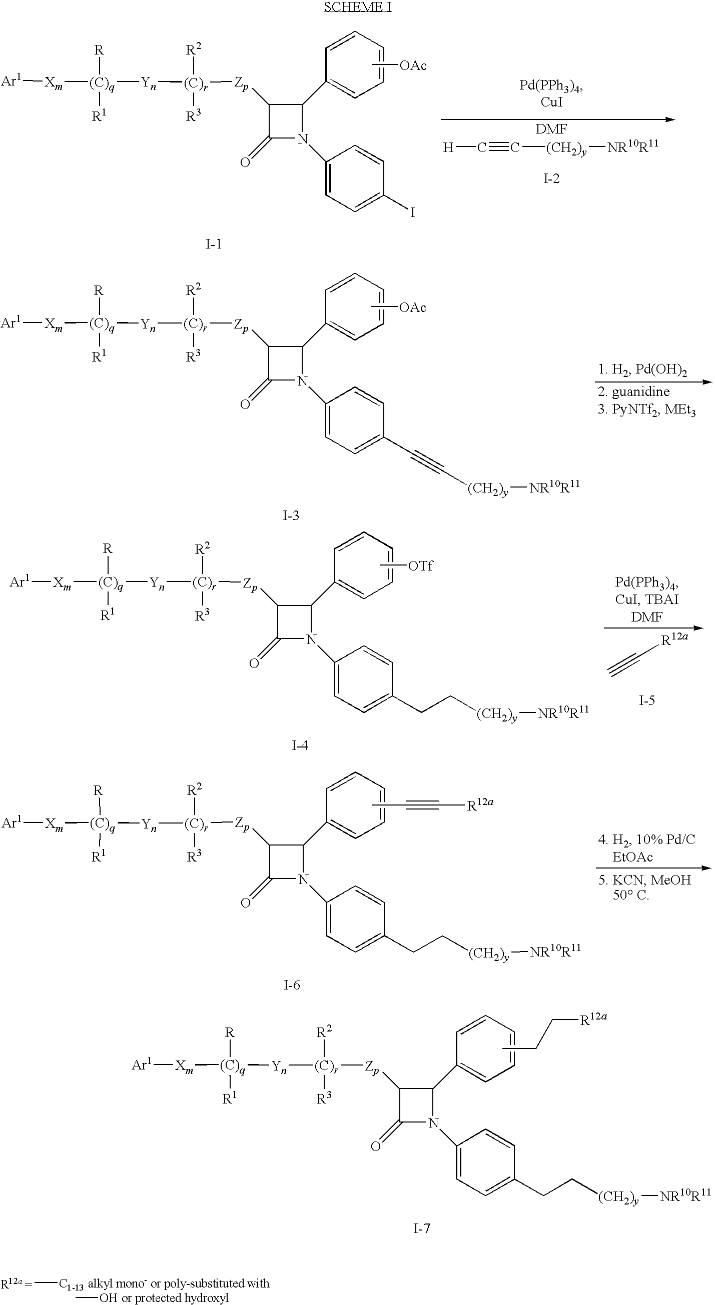

The preparation of compounds possessing a 2-hydroxyphenyl group in the final product I-12 is outlined in Scheme II. The bis(benzyloxy)intermediate I-8 may be treated with a terminal alkyne of type I-2 in the presence of a suitable palladium catalyst such as tetrakistriphenylphosphine palladium(0) or [1,1′-bis(diphenylphosphino)ferrocene]dichloropalladium(II) or the like, and copper(I) iodide. The reaction is usually performed in an inert organic solvent such as DMF, between room temperature and 100° C., for a period of 6-48 h, and the product is an internal alkyne of structural formula I-9. Alkyne I-2 may contain a radioactive atom such as ³⁵S to provide the corresponding radiolabeled adduct upon reaction with I-8. Conversion of I-9 to I-10 can be achieved by hydrogenation of the triple bond, with concomitant selective hydrogenolysis of the benzyl ether which is not at the 2-position, followed by converting the resulting phenol to the triflate I-10 via treatment with triflic anhydride (trifluoromethanesulfonic acid anhydride) in the presence of pyridine in dichloromethane medium. The remaining steps can be performed as described in Scheme I.

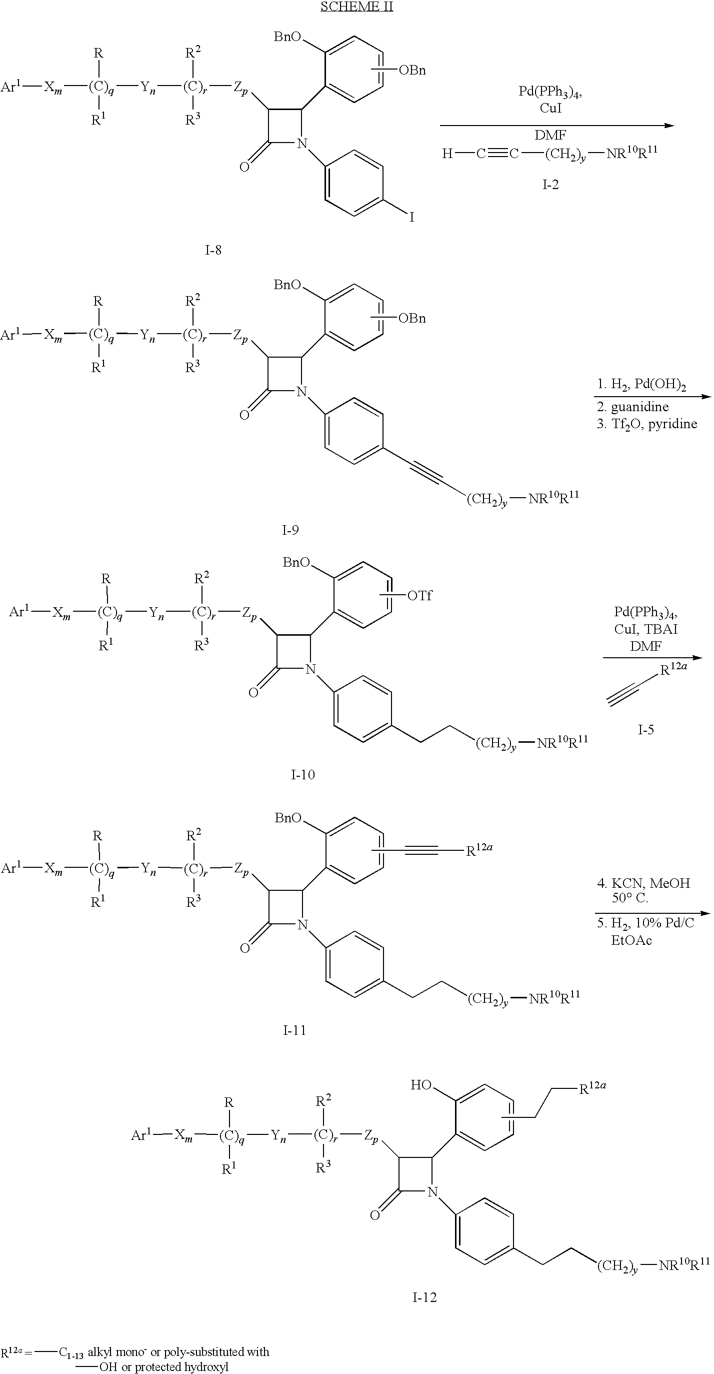

As shown in Scheme III, incorporation of the trihydroxyl alkyl group directly to the phenyl ring may be achieved by palladium assisted coupling of an intermediate such as triflate I-13 with an alkenyl stannyl intermediate, such as I-14, n=0. The reaction may be performed in an inert organic solvent such as DMF or toluene with heating for a period of 1-24 h in the presence of a palladium catalyst such as PdCl₂(PPh₃)₂ or tetrakistriphenylphosphine palladium(0) to afford the product I-15 (n=0). Dihydroxylation of this alkenyl phenyl intermediate may be achieved using standard conditions such as OsO4 (catalytic) with N-Methylmorpholine N-oxide re-oxidant in the presence of a base such as triethylamine in an appropriate solvent. Removal of any protecting groups if present, e.g. benzyl protecting groups, may be achieved by treatment with 10% palladium on carbon catalyst under hydrogen atmosphere in a solvent such as ethyl acetate over 15-24 hours to afford compounds of formula I-16 (n=0). Alternatively, if the intermediate contains protecting groups such as acetate, deprotection to afford the free hydroxyl groups may be achieved as described previously using KCN in MeOH. Products of formula I-15 and I-16 with a one carbon linker to the phenyl ring (n=1) can similarly be prepared following this reaction sequence using the alkenyl stannyl intermediate, I-14 wherein n=1.

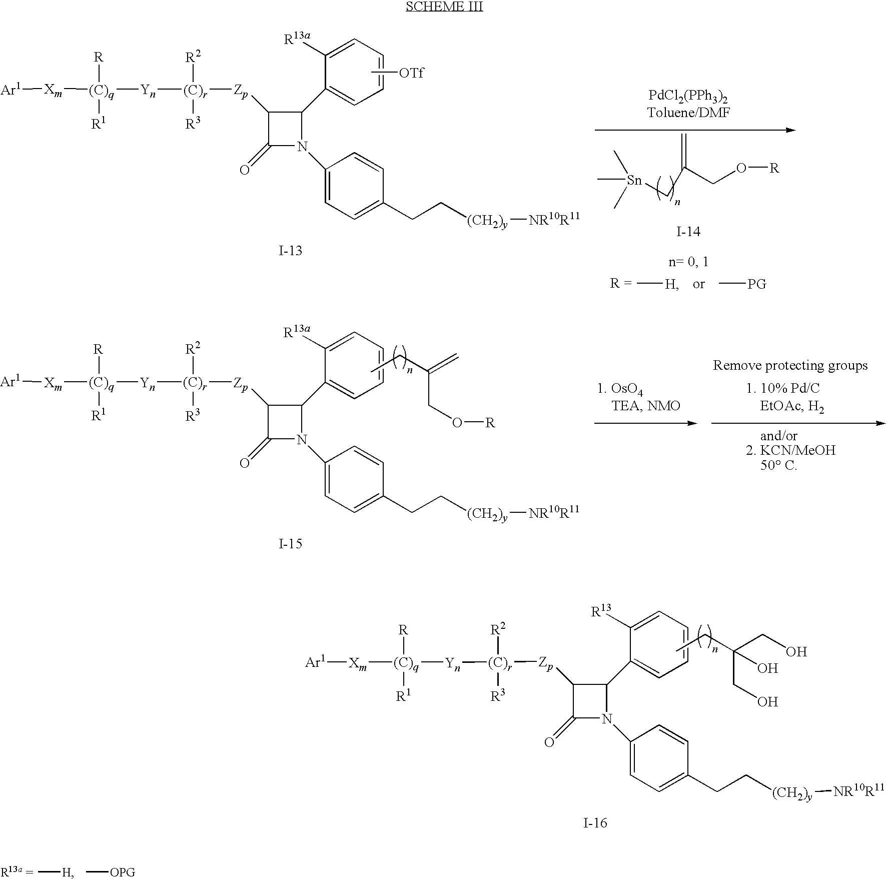

As shown in Scheme IV, compounds containing a 2-carbon linker to the functionalized nitrogen group may be obtained by treating the alkenyl intermediate I-17 with 9-borabicyclo[3.3.1]nonane (9-BBN) to form the alkyl borate ester, which upon palladium catalyzed cross-coupling with the iodide I-18 may afford the intermediate I-19 possessing a 2-carbon-linked nitrogen functional group. Intermediate I-19 may be deprotected and then converted to functionalized nitrogen intermediates using procedures as described herein and those known in the art for sulfonamide formation, carboxamide formation, etc. Subsequent intermediates may then be converted to compounds of the present invention using procedures similar to those previously described above and in Schemes I, II and III.

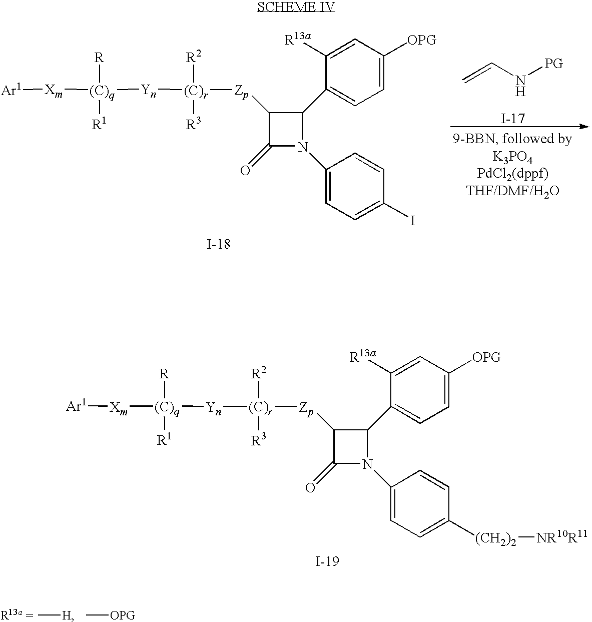

As shown in Scheme V, in a related fashion, compounds containing a one carbon linker may be obtained by treating iodo intermediate I-18 with reagents capable of aryl cyanation such as trimethylsilylcyanide (TMS-CN) and a palladium catalyst to afford aryl cyanide intermediates. This cyano-intermediate may be hydrogenated in the presence of Raney-Nickel catalyst to afford the desired aminomethyl intermediate I-19 with one carbon-linked nitrogen group. This intermediate may then be converted to functionalized nitrogen intermediates using procedures as described herein and those known in the art for sulfonamide formation, carboxamide formation, etc. Further manipulation of compounds of formula I-19 may be achieved by sequence similar to those described in Schemes I-III to make compounds of Formula I.

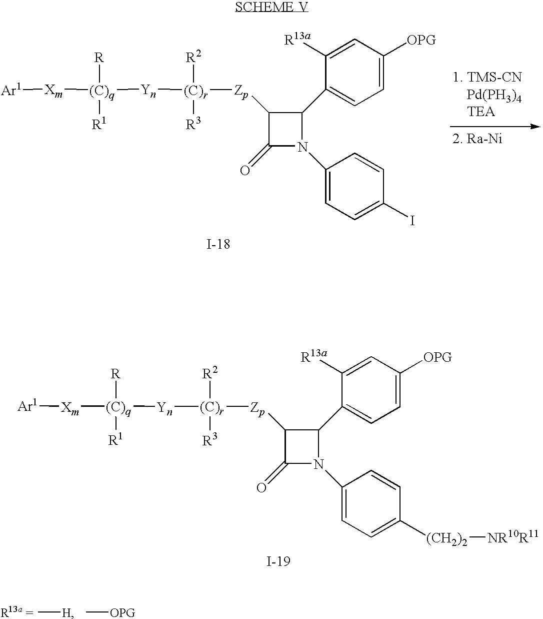

The following examples are provided to illustrate the invention and are not to be construed as limiting the scope of the invention in any matter. Within the following synthetic examples, reference to an intermediate from a prior step is a reference to an intermediate compound made in a prior step within the same example, unless otherwise noted. The following designations are used in the Examples for certain repetitively used intermediates:

### Preparation of N-prop-2-yn-1-ylmethanesulfonamide (i-1)

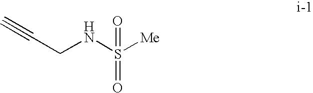

Methansulfonylchloride (1.40 mL, 18.1 mmol) was added dropwise to a stirred solution of propargylamine (1.00 g, 18.1 mmol) and dimethylaminopyridine (44.0 mg, 0.36 mmol) in pyridine (10 mL) at 0° C. After aging for approximately 15 h, the reaction mixture was poured into 1N HCl and extracted twice with ethyl acetate. The combined organic extracts were washed with saturated aqueous sodium bicarbonate, brine, dried (MgSO₄), filtered and concentrated in vacuo, to afford the title compound i-1. Crude i-1 crystallized on standing and was used without further purification. ¹HNMR (500 MHz, CDCl₃) δ: 4.92 (br s, 1H), 3.99 (dd, J=2.3, 6.2 Hz, 2H), 3.11 (s, 3H), 2.70 (br t, J=2.3 Hz).

### Preparation of N-Methyl-N-prop-2-yn-1-ylmethanesulfonamide (i-2)

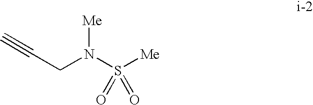

Methanesulfonylchloride (1.12 mL, 14.5 mmol) was added to a stirred solution of N-methylpropargylamine (1.22 mL, 14.5 mmol) and dimethylaminopyridine (35 mg, 0.30 mmol) in pyridine (10 mL) at room temperature. After aging for approximately 15 h, the reaction mixture was poured into ethyl acetate and washed successively with 1N HCl and brine. The organic phase was dried (Na₂SO₄), filtered and concentrated in vacuo, to afford the title compound (i-2), which was used without further purification.

### Preparation of N-prop-2-yn-1-ylacetamide (i-3)

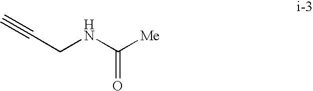

Acetyl chloride (0.52 mL, 7.3 mmol) was added to a stirred solution of propargylamine (0.5 mL, 7.3 mmol) and dimethylaminopyridine (18 mg, 0.14 mmol) in pyridine (2.5 mL) at 0° C., and the resulting mixture was allowed to warm to ambient temperature. After approximately 15 h, the reaction mixture was diluted with ethyl acetate and washed successively with 1N HCl and brine. The organic phase was dried (Na₂SO₄), filtered and concentrated in vacuo to afford the title compound (i-3), which was used without further purification.

### Preparation of N-prop-2-yn-1-ylbenzenesulfonamide (i-4)

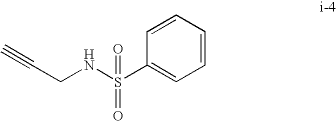

Benzene sulfonyl chloride (1.16 mL, 9.1 mmol) was added to stirred solution of propargylamine (0.62 mL, 9.1 mmol) and dimethylaminopyridine (22 mg, 0.18 mmol) in pyridine (5 mL) at room temperature. The resulting solution was aged at ambient temperature for approximately 15 h. The reaction mixture was diluted with ethyl acetate and washed successively with 1N HCl and brine. The organic phase was dried (Na₂SO₄), filtered and concentrated in vacuo to furnish the title compound (i-4), which was used without further purification.

### Preparation of N,N-Dimethyl-N′-prop-2-yn-1-ylurea (i-5)

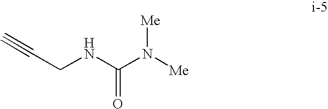

Dimethyl carbamylchloride (0.84 mL, 9.1 mmol) was added to a stirred solution of propargylamine (0.62 mL, 9.1 mmol) and dimethylaminopyridine (22 mg, 0.18 mmol) in pyridine (5 mL) at room temperature. The resulting suspension was stirred at ambient temperature for approximately 15 h. The reaction mixture was diluted with ethyl acetate and washed successively with 1N HCl and brine. The organic phase was dried (Na₂SO₄), filtered and concentrated in vacuo to afford the title compound (i-5), which was used without further purification.

### Preparation of 5-ethynyl-2,2-dimethyl-1,3-dioxan-5-yl acetate (i-6)

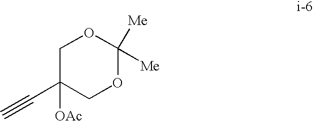

To a dry 250 mL round bottom flask was charged with a 0.5M solution of ethynylmagnesium bromide in THF (115 mL, 57.7 mmol) under nitrogen atmosphere. The resulting solution was cooled to 0° C. in an ice bath. To the cooled solution was added slowly a solution of 2,2-dimethyl-1,3-dioxane-5-one (5 g, 38.44 mmol) in 50 mL dry THF. The ice bath was removed and the resulting reaction mixture was stirred at ambient temperature for 1.5 hrs. The reaction mixture was quenched with sat. aq. NH₄Cl (50 mL) and then extracted with ethyl acetate (100 mL). The organic layer was dried over Na₂SO₄, filtered and the solvent removed under vacuum to afford the crude intermediate.

The crude intermediate was dissolved in CH₂Cl₂ (100 mL) under nitrogen atmosphere. To the resulting solution was added simultaneously by syringe acetic anhydride (4.34 mL, 46 mmol) and TEA (6.4 mL, 46 mmol). To the reaction mixture was added DMAP (0.56 g, 4.6 mmol). The reaction mixture was stirred for 3 hrs at room temperature at which time the reaction was quenched by the addition of 1N aq. HCl (100 mL). The reaction mixture was transferred to separatory funnel and the organic layer was separated. The organic layer was washed with aq. NaHCO₃ (100 mL), water (50 mL), brine, dried, filtered and the solvent removed under vacuum to afford the title compound (i-6) which was used without further purification. ¹HNMR (500 MHz, CDCl₃) δ: 4.14 (d, J=12.6, 2H) 4.07 (d, J=12.6 Hz, 2H), 2.65 (s, 1H), 2.12 (s, 3H), 1.45 (s, 3H), 1.41 (s, 3H).

The compound (3R,4S)-3-[(3S)-3-(4-fluorophenyl)-3-hydroxypropyl]-4-(4-hydroxyphenyl)-1-(4-iodophenyl)azetidin-2-one (i-7) and (i-7a) were prepared according to Burnett, D. S.; Caplen, M. A.; Domalski, M. S.; Browne, M. E.; Davis, H. R. Jr.; Clader, J. W. Bioorg. Med. Chem. Lett. (2002), 12, 311. Compound i-8 is the dihydroxy-protected analog of i-7, where the protecting groups are acetyl.

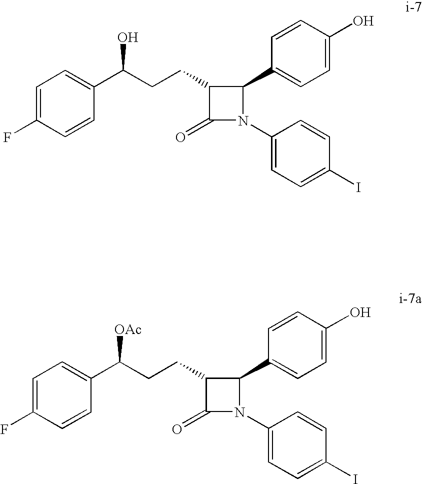

### Preparation of 4-[(2S,3R)-3-[(3S)-3-(acetyloxy)-3-(4-fluorophenyl)propyl]-1-(4-iodophenyl)-4-oxoazetidin-2-yl]phenyl acetate (i-8)

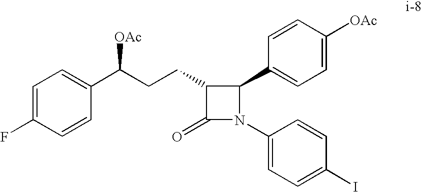

To a solution of (1S)-1-(4-fluorophenyl)-3-[(2S,3R)-2-(4-hydroxyphenyl)-1-(4-iodophenyl)-4-oxoazetidin-3-yl]propyl acetate (1-7a) (2 g, 3.58 mmol) (prepared according to Burnett, D. S.; Caplen, M. A.; Domalski, M. S.; Browne, M. E.; Davis, H. R. Jr.; Clader, J. W. Bioorg. Med. Chem. Lett. (2002), 12, 311) in CH₂Cl₂ (25 mL) under nitrogen atmosphere was added acetic anhydride (0.4 mL, 4.30 mmol), triethylamine (0.75 mL, 5.38 mmol) and DMAP. The reaction mixture was stirred at RT for 1 hr and the solvent removed under vacuum. The residue was purified by MPLC (silica column) with stepwise gradient elution; (0-100% EtOAc/hexanes as eluent) to afford the title compound (i-8). m/z (ES) (M-OAc)⁺. ¹HNMR (500 MHz, CDCl₃) δ: 7.57 (d, J=8.6, 1H) 7.38-7.26 (m, 5H), 7.22 (br d, J=7.1H, 2H), 7.14 (d, J=8.5 Hz, 1H), 7.08-7.02 (m, 3H), 5.74 (t, J=6.7 Hz, 1H), 4.62 (d, J=2.3 Hz, 1H), 3.10 (dt, J=2.3, 7.8 Hz, 1H), 2.34 (s, 3H), 2.08 (s, 3H), 2.09-2.03 (m, 2H), 1.94-1.86 (m, 2H).

Additional intermediates described in the Examples:

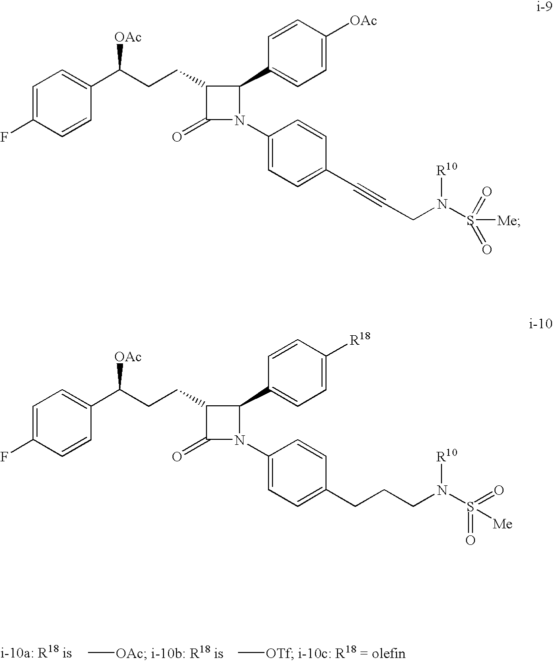

### Preparation of [(hex-5-yn-1-yloxy)methyl]benzene or benzyl hex-5-yn-1-yl ether (i-11)

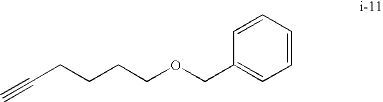

To a solution of 5-hexyn-1-ol (1.17 g, 11.88 mmol) in anhydrous DMF (100 mL) under nitrogen atmosphere was added TBAI (0.87 g, 2.38 mmol) followed by 60% NaH dispersion in oil (0.55 g, 14.26 mmol) in portions over 0.5 h. The reaction mixture was stirred for 0.5 hr at which time benzyl bromide (2.44 g, 14.26 mmol) was added by syringe. The reaction mixture was stirred for 16 h at room temperature at which time the reaction was quenched by the addition of sat. aq. NH₄Cl (100 mL). The reaction mixture was transferred to separatory funnel and extracted with ether (3×75 mL). The combined organic extracts were washed with water (50 mL), brine (75 mL), dried (Na₂SO₄), filtered and the solvent removed under vacuum. The residue was purified by MPLC (silica column) with stepwise gradient elution (0-60% EtOAc/hexanes as eluent) to afford the title compound (i-11).

(1S)-3-[(2S,3R)-2-[2,4-bis(benzyloxy)phenyl]-1-(4-iodophenyl)-4-oxoazetidin-3-yl]-1-(4-fluorophenyl)propyl acetate (i-12) was prepared from 2,4-bisbenzyloxyacetaldehyde and 4-iodoaniline using procedures as described in Vaccaro, W. D. et al., Bioorg. Med. Chem., vol. 6 (1998), 1429-1437.

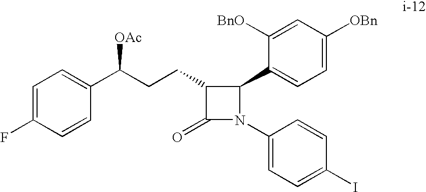

### Preparation of 4-(methylsulfonyl)but-1-yne

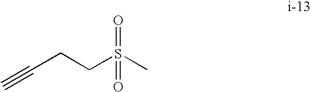

A solution of 3-butyn-1-ol (1000 mg; 14.27 mmol) and methanesulfonyl chloride (1.63 g, 14.27 mmol) in dichloromethane (35 ml) was cooled in a bath to 0° C. and to this solution triethylamine (2.09 ml, 14.98 mmol) in dichloromethane (5 ml) was added drop by drop over about 5 minutes. The resulting reaction mixture was stirred vigorously for 0.5 h at 0° C. and then the stirring was continued for a further 0.5 h at room temperature. The volatiles were removed on a rotary evaporator under reduced pressure and the residues left were partitioned between diethyl ether (2×50 ml) and 1N hydrochloric acid (50 ml). The combined ethereal extracts were dried over anhydrous magnesium sulfate powder, filtered and the resulting filtrates concentrated under reduced pressure to leave a pale yellow mobile liquid which was the but-3-yn-1-yl methanesulfonate ester.

To a solution of the crude but-3-yn-1-yl methanesulfonate (0.5 g, 3.37 mmol) in ethanol (7.5 ml) was added sodium thiomethoxide (248 mg, 3.54 mmol) powder in small batches over about 5 minutes and the resulting mixture stirred under an inert atmosphere for 12 h at room temperature. A few drops of distilled water were added to dissolve up the cloudy solution and give a faint yellow homogeneous solution. A peracetic acid solution was prepared from 30% aqueous hydrogen peroxide (3 ml), acetic acid (5 ml) and 3 drops of conc. sulfuric acid at 0° C. A portion of this peracid solution (3 ml) was added cautiously to the ethanol solution and the reaction mixture was stirred at room temperature for 8 h, then concentrated on a rotary evaporator and the oily residues obtained were partitioned with dichloromethane (3×25 ml) and water. The combined dichloromethane extracts were washed with saturated sodium carbonate solution added to neutralize the acid (tested with pH paper) and with saturated sodium sulfite solution to remove excess oxidant (until negative to starch iodide paper). The dichloromethane layer was dried over anhydrous magnesium sulfate powder, filtered and the filtrates concentrated under reduced pressure. The oil which remained on evaporation was purified on preparative tlc plates that were eluted with dichloromethane:Methanol (97:3 v/v) to give the product 4-(methylsulfonyl)but-1-yne (139.2 mg) as a mobile liquid. ¹H-NMR (400 MHz, CD₃OD) δ: 3.19 (t, J=7 Hz, 2H), 2.98 (s, 3H), 2.74 (dt, J=7, 2.5 Hz, 2H), 2.13 (t, J=2.5 Hz, 1H).

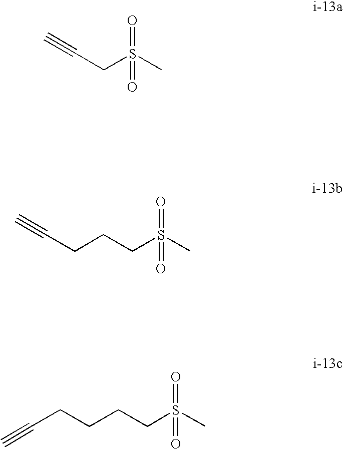

Intermediates related to those described above of varying chain length may be prepared from the appropriate starting materials using the procedures described above.

### Example 1

### N-[3-(4-{(2S,3R)-2-{4-[3,4-dihydroxy-3-(hydroxymethyl)butyl]phenyl}-3-[(3S)-3-(4-fluorophenyl)-3-hydroxypropyl]-4-oxoazetidin-1-yl}phenyl)propyl]methanesulfonamide

### Step A: Preparation of 4-[(2S,3R)-3-[(3S)-3-(acetyloxy)-3-(4-fluorophenyl)propyl]-1-(4-{3-[(methylsulfonyl)amino]prop-1-yn-1-yl}phenyl)-4-oxoazetidin-2-yl]phenyl acetate

### (i-9 wherein R¹⁰ is —H)

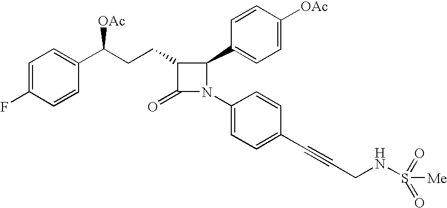

Dichlorobis(triphenylphosphine)palladium(II) (1.27 g, 1.68 mmol) and copper(I) iodide (632 mg, 3.32 mmol) were added to a solution of i-8 (10.0 g, 16.6 mmol) and i-1 (3.34 g, 25.0 mmol) in triethylamine (16.2 mL, 116.34 mmol) and DMF (150 mL). The reaction mixture was saturated with nitrogen and stirred at room temperature. After 2 h, the reaction mixture was partitioned between 400 mL EtOAc and 250 mL water. The organic layer was washed with water (150 mL), brine (150 mL), dried (MgSO₄), filtered and concentrated in vacuo. Purification of the crude residue by MPLC (silica column) with stepwise gradient elution; (0-100% EtOAc/hexanes as eluent) afforded the title compound. m/z (ES) 629 (M+Na)⁺, 547 (M-OAc)⁺. ¹HNMR (500 MHz, CDCl₃) δ: 7.35 (d, J=8.4 Hz, 1H), 7.28 (dd, J=6.4, 8.4 Hz, 1H), 7.19 (d, J=8.5 Hz, 1H), 7.12 (d, J=8.5 Hz, 1H), 7.08 (d, J=8.3 Hz, 1H), 7.02 (dd, J=6.5, 8.6 Hz, 1H), 5.72 (t, 6.6 Hz, 1H), 4.60 (d, J=2.3 Hz, 1H), 4.21-4.16 (m, 1H), 4.15 (overlapped dd, J=7.1, 11 Hz, 1H), 3.15-3.12 (m, 2H), 3.09-3.04 (m, 1H), 2.96 (s, 3H), 2.58 (t, 7.6 Hz, 2H), 2.30 (s, 3H), 2.07 (overlapped s, 3H), 2.09-2.03 (m, 2H), 1.90-1.83 (m, 4H).

### Step B: Preparation of 4-[(2S,3R)-3-[(3S)-3-(acetyloxy)-3-(4-fluorophenyl)propyl]-1-(4-{3-[(methylsulfonyl)amino]propyl}phenyl)-4-oxoazetidin-2-yl]phenyl acetate (i-10a wherein R¹⁰ is —H)

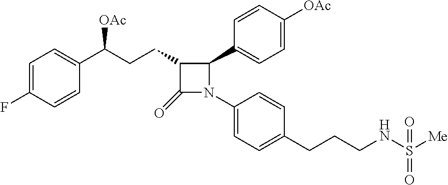

A mixture of the intermediate from Step A (8.5 g, 14 mmol) and 10% palladium on activated carbon (2.2 g) in ethanol (100 mL) and EtOAc (150 mL) was hydrogenated at atmospheric pressure. After 15 h, the reaction mixture was filtered through MgSO₄ and filter aid and the filtered catalyst washed several times with EtOAc. The filtrate was concentrated in vacuo to afford the title compound which was used without further purification. m/z (ES) 663 (M+Na)⁺, 551 (M-OAc)⁺.

### Step C: Preparation of (1S)-1-(4-fluorophenyl)-3-[(3R,4S)-1-(4-{3-[(methylsulfonyl)amino]propyl}phenyl)-2-oxo-4-(4-{[(trifluoromethyl)-sulfonyl]oxy}phenyl)azetidin-3-yl]propyl acetate (i-10b wherein R¹⁰ is —H)

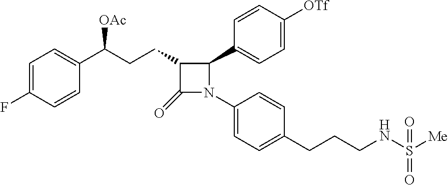

Guanidine hydrochloride (1.34 g, 13.93 mmol) was added to a mixture of the intermediate from Step B, (8.5 g, 13.93 mmol) and triethylamine (1.95 mL, 13.93 mmol) in methanol (150 mL). After 3 h, the solvent was removed under vacuum and the residue was dissolved in EtOAc (200 mL)/water (100 mL) and 2N aq. HCl. The mixture was transferred to a separatory funnel and the layers separated. The organic layer was washed with brine (100 mL), dried (MgSO₄), filtered and concentrated in vacuo to afford a clear oil.

The crude intermediate was dissolved in methylene chloride (100 mL) and to the solution was added (bis(trifluoromethylsulfonyl)amino pyridine (8.14 g, 13.93 mmol), triethylamine (1.95 mL, 13.93 mmol), DMAP (˜100 mg, catalytic). The resulting solution was stirred for 2 h at room temperature. The reaction was quenched with 1N aq. HCl and the organic layer was separated. The organic extract was washed with brine, dried (MgSO₄) and concentrated in vacuo. Purification of the crude residue by MPLC (silica column) with stepwise gradient elution (0-100% EtOAc/hexanes as eluent) afforded the title compound. m/z (ES) 723 (M+Na)⁺, 641 (M-OAc)⁺.

### Step D: Preparation of (1S)-3-[(2S,3R)-2-(4-{[5-(acetyloxy)-2,2-dimethyl-1,3-dioxan-5-yl]ethynyl}phenyl)-1-(4-{3-[(methylsulfonyl)amino]propyl}phenyl)-4-oxoazetidin-3-yl]-1-(4-fluorophenyl)propyl acetate

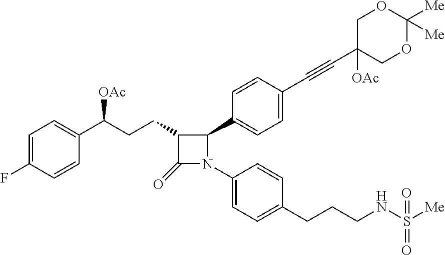

To an oven dried flask 250 mL flask was added CuI (300 mg, 1.44 mmol), tetrabutylammonium iodide (TBAI, 1.58 g, 4.28 mmol). The charged flask was set under nitrogen atmosphere and a solution of the intermediate from Step C, (3.5 g, 4.28 mmol) in 30 mL anhydrous DMF was added to the flask. A solution of 5-ethynyl-2,2-dimethyl-1,3-dioxan-5-yl acetate (i-6) (1.70 g, 8.56 mmol) in DMF (20 mL) was added to the mixture. The flask was then equipped with a condensor, and the mixture was evacuated and set under nitrogen several times to degas the solvent. Solid Pd(PPh₃)₄ (3.32 g, 3 mmol) was then added to the reaction followed by TEA (4.2 mL, 30 mmol). The reaction mixture was heated to 70° C. for 2 hours during which time the reaction mixture became dark brown in color. The reaction was removed from the heating bath, cooled and partitioned with EtOAc (250 mL) and 1N aq. HCl (100 mL). The organic layer was washed with water (100 mL), brine (75 mL), dried over magnesium sulfate, filtered and concentrated under vacuum. The residue was purified by MPLC (silica column) with stepwise gradient elution; (0-100% EtOAc/hexanes as eluent) to afford the title compound. m/z (ES) 689 (M-OAc)⁺. ¹HNMR (500 MHz, CD₃OD) δ: 7.44 (d, J=8.3 Hz, 1H), 7.38-7.32 (m, 4H), 7.16 (d, J=8.5 Hz, 2H), 7.10 (d, J=8.5 Hz, 2H), 7.06 (t, J=8.6 Hz, 2H), 5.70 (app t, 6.3 Hz, 1H4.20 (s, 3H), 3.10-3.05 (m, 1H), 3.02 (d, J=7.0 Hz, 2H), 2.89 (s, 3H), 2.60 (t, 7.4 Hz, 2H), 2.10 (s, 3H), 2.04 (s, 3H), 1.78 (t, J=7.6, 3H), 1.47 (s, 3H), 1.39 (s, 3H).

### Step E: Preparation of (1S)-3-[(2S,3R)-2-(4-{2-[5-(acetyloxy)-2,2-dimethyl-1,3-dioxan-5-yl]ethyl}phenyl)-1-(4-{3-[(methylsulfonyl)amino]propyl}phenyl)-4-oxoazetidin-3-yl]-1-(4-fluorophenyl)propyl acetate

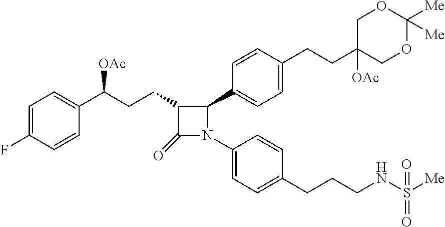

A roundbottom flask was charged with 10% Pd—C (500 mg) and 300 mg 20% Pd(OH)₂—C. EtOAc (˜2 mL) was added to cover the solid catalyst mixture. To this mixture was added a solution of the intermediate from Step D, (1.5 g, 2.0 mmol) in ethanol (40 mL) and ethyl acetate (2 mL). The resulting suspension set under hydrogen atmosphere and stirred vigorously for 1 hr. The catalysts were filtered, solids washed with ethanol and the solvent was removed under vacuum to obtain partially hydrogenated intermediate. The reaction procedure was repeated as above. A round bottom flask was charged with 10% Pd—C (500 mg) and 300 mg 20% Pd(OH)₂—C. EtOAc (˜2 mL) was added to cover the solid catalyst mixture. To this mixture was added a solution of the intermediate from above in ethanol (40 mL) and ethyl acetate (2 mL). The resulting suspension set under hydrogen atmosphere and stirred vigorously for 2 hours. The catalyst was filtered through filter aid and MgSO₄ and washed with EtOH/EtOAc. The filtrate was concentrated in vacuo to afford the title compound which was used without further purification. m/z (ES) 692 (M-OAc)⁺. ¹HNMR (500 MHz, CD₃OD) δ: 7.31-7.24 (m, 6H), 7.21-7.17 (m, 3H), 7.08-7.02 (m, 3H), 5.72 (app t, 6.7 Hz, 1H) 4.60 (d, J=2.1 Hz, 1H), 4.20 (app t, J=6.5, 1H), 4.02 (d, J=12.4 Hz, 2H), 3.90 (d, J=12.2 Hz, 2H), 3.13 (q, J=6.7 Hz, 2H), 3.06 (dt, J=2.2, 7.6 Hz, 1H), 2.94 (s, 3H), 2.60 (app q, 7.4 Hz, 4H), 2.35-2.29 (m, 2H), 2.08 (s, 3H), 2.03 (s, 3H), 1.83-1.90 (m, 3H), 1.45 (s, 3H), 1.40 (s, 3H).

### Step F: Preparation of 3-{4-[(2S,3R)-3-[(3S)-3-(acetyloxy)-3-(4-fluorophenyl)propyl]-1-(4-{3-[(methylsulfonyl)amino]propyl}phenyl)-4-oxoazetidin-2-yl]phenyl}-1,1-bis(hydroxymethyl)propyl acetate

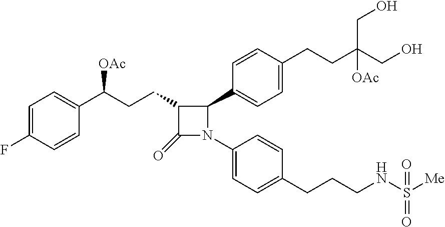

To a solution of the intermediate of step E (1.5 g, 2 mmol) in THF/water (16 mL/4 mL) was added TFA (1 mL). The reaction mixture was stirred at RT for 16 hr. To the reaction mixture was added 100 mL toluene and the water was removed under vacuum with water bath temperature of 40° C. The residue was treated twice with 100 mL toluene followed by azeotropic removal of water. The solvent was completely removed under vacuum. The crude product was purified by MPLC (silica column) with stepwise gradient elution (50-100% EtOAc/hexanes as eluent). Mixed fractions were also isolated and were further purified by prep TLC eluting with CH₂Cl₂/MeOH (95/5). The purified fractions were combined to afford the title compound. m/z (ES) 653 (M-OAc)⁺.

### Step G: Preparation of N-[3-(4-{(2S,3R)-2-{4-[3,4-dihydroxy-3-(hydroxymethyl)butyl]phenyl}-3-[(3S)-3-(4-fluorophenyl)-3-hydroxypropyl]-4-oxoazetidin-1-yl}phenyl)propyl]methanesulfonamide

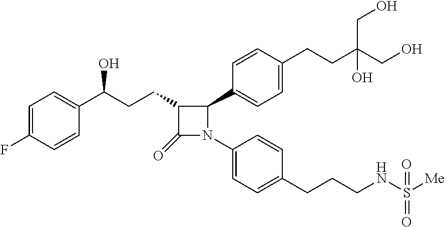

To a solution of the intermediate from Step F, (1.05 g, 1.47 mmol) in methanol (2.5 mL) was added potassium cyanide (100 mg, 1.58 mmol) and the resulting solution stirred at 50° C. for 2 hours. The solution was concentrated and the residue purified by preparative TLC plate eluting with methanol/dichloromethane (10/90) to afford the title compound. This product was further purified by MPLC (silica column) with stepwise gradient elution; (5-10% EtOH/EtOAc as eluent) to afford the title compound. m/z (ES) 611 (M-OAc)⁺ and 651 (M+Na)⁺¹HNMR (500 MHz, CD₃OD) δ: 7.35-7.31 (m, 2H), 7.28-7.234 (m, 4H), 7.18 (d, J=8.5 Hz, 2H), 7.10 (d, J=8.6 Hz, 2H), 7.03 (app, t, J=8.6 Hz, 2H), 4.79 (br d, J=2.1 Hz, 1H), 4.60 (br dd, J=5.1, 6.60 Hz, 1H), 3.53 (s, 4H), 3.09-3.03 (m, 1H), 3.02 (t, J=6.8 Hz, 2H), 2.88 (s, 3H), 2.73-2.67 (m, 2H), 2.61 (t, 7.6 Hz, 2H), 1.97-1.83 (m, 3H), 1.81-1.73 (m, 3H).

### Example 2

### N-[3-(4-{(2S,3R)-2-{4-[1,2-dihydroxy-1-(hydroxymethyl)ethyl]phenyl}-3-[(3s)-3-(4-fluorophenyl)-3-hydroxypropyl]-4-oxoazetidin-1-yl}phenyl)propyl]methanesulfonamide

### Step A: Preparation of Trimethyl{[2-(tributylstannyl)prop-2-en-1-yl]oxy}silane

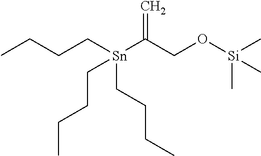

To a solution of trimethylsilylpropyne (1 g, 7.80 mmol) in anhydrous THF (35 mL) was added Dichloro-Bis(triphenylphosphine)palladium (44 mg, 0.06 mmol) and the resulting solution was set under nitrogen atmosphere and cooled to 0° C. using an ice/water bath. Once the internal temperature was 0° C., tributyltin (3.10 mL, 11.70 mmol) was added dropwise to the solution via syringe and the resulting mixture stirred at 0° C. for 45 minutes. Solvent was evaporated in vacuo and the residue purified by flask column eluting with 99/1 hexane/triethylamine to afford the title compound. ¹HNMR (500 MHz, CDCl₃) δ: 5.91 (dd, J=1.8, 3.9 Hz, 1H), 5.27 (dd, J=1.6, 2.1 Hz, 1H), 4.31 (br s, 2H), 1.60-1.44 (m, 8H), 1.39-1.1.31 (m, 8H), 0.99-0.94 (m, 20H).

### Step B: Preparation of (1S)-1-(4-fluorophenyl)-3-[(2S,3R)-2-{4-[1-(hydroxymethyl)vinyl]phenyl-1-(4-{3-[(methylsulfonyl)amino]propyl}phenyl)-4-oxoazetidin-3-yl]propyl acetate

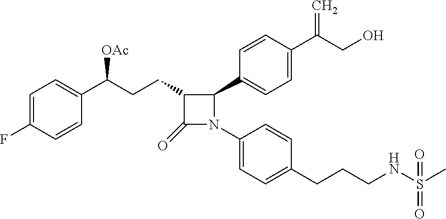

An oven-dried 50 mL round bottom flask was charged with lithium chloride (35 mg, 0.84 mmol) and set under vacuum to remove any trace of water from the salt. After 10 minutes, nitrogen atmosphere was introduced followed by the addition of palladium tetrakis (16 mg, 0.014 mmol) and copper chloride (69 mg, 0.705 mmol) to the flask. The materials were then degassed with nitrogen purging three times. DMSO (dimethyl sulfoxide) (1 mL) was then added followed by the intermediate (i-10b wherein R¹⁰ is —H) (100 mg, 0.14 mmol) and the tin reagent from Step A, (71 mg, 0.17 mmol). The resulting mixture was heated to 60° C. for three hours and then cooled. The solution was diluted with ethyl acetate and washed with water (2×2 mL) and brine (2 mL). The organics were dried over magnesium sulfate, filtered and then evaporated in vacuo. Preparative plate purification eluting with 70% ethyl acetate/30% hexane afforded the title compound. The TMS protecting group was removed during the reaction. m/z (ES) 609 (M+H)⁺, 549 (M-OAc)⁺.

### Step C: Preparation of (1S)-3-[(2S,3R)-2-{4-[1,2-dihydroxy-1-(hydroxymethyl)ethyl]phenyl-1-(4-{3-[(methylsulfonyl)amino]propyl}phenyl)-4-oxoazetidin-3-yl]1-(4-fluorophenyl)propyl acetate

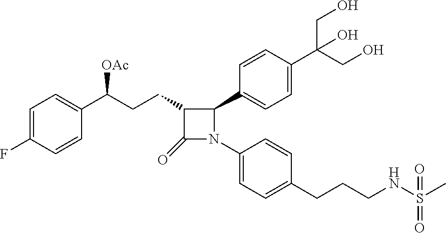

To a solution of the intermediate from step B, (35 mg, 0.057 mmol) in 8:1 acetone/water (1 mL) was added N-methyl morpholine-N-oxide (12 mg, 0.114 mmol) followed by OsO₄ (2.5% solution in isopropanol, 50 μL, 0.001 mmol) and the resulting mixture stirred for 3 hours at room temperature. The solution was diluted with dichloromethane (10 mL) and washed with 1N HCl (3 mL) followed by water (3 mL) and Brine (3 mL). The organics were dried over magnesium sulfate, filtered and evaporated in vacuo. Preparative plate purification eluting with 5% methanol/95% dichloromethane afforded the title compound. m/z (ES) 665 (M+Na)⁺, 583 (M-OAc)⁺.

### Step D: Preparation of N-[3-(4-{(2S,3R)-2-{4-[1,2-dihydroxy-1-(hydroxymethyl)ethyl]phenyl}-3-[(3S)-3-(4-fluorophenyl)-3-hydroxypropyl]-4-oxoazetidin-1-yl}phenyl)propyl]methanesulfonamide

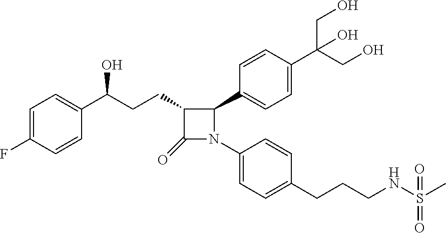

To a solution of the intermediate from Step C, (25 mg, 0.039 mmol) in methanol (1 mL) was added potassium cyanide (2 mg, 0.035 mmol) and the resulting solution stirred at 50° C. for 2 hours. The solution was concentrated and the residue purified by preparative TLC plate eluting with methanol/dichloromethane (10/90) to afford the title compound. m/z (ES) 623 (M+Na)⁺, 583 (M-OH)⁺. ¹HNMR (500 MHz, CD₃OD) δ: 7.56 (d, J=8.3 Hz, 2H), 7.37 (d, J=8.3 Hz, 2H), 7.32 (dd, J=5.5, 8.4 Hz, 2H), 7.208 (d, J=8.5 Hz, 2H), 7.10 (d, J=8.4 Hz, 2H), 7.04 (app, t, J=8.7 Hz, 2H), 4.86 (br d, J=2.3 Hz, 1H), 4.61 (br app t, J=6.6 Hz, 1H), 3.76 (br s, 4H), 3.08-3.04 (m, 1H), 3.02 (t, J=6.8 Hz, 2H), 2.88 (s, 3H), 2.60 (t, 7.6 Hz, 2H), 1.98-1.84 (m, 3H), 1.81-1.75 (m, 3H).

### Example 3

### N-[3-(4-{(2S,3R)-2-{4-[2,3-dihydroxy-2-(hydroxymethyl)propyl]phenyl}-3-(3S)-3-(4-fluorophenyl)-3-hydroxypropyl]-4-oxoazetidin-1-yl}phenyl)propyl]methanesulfonamide

### Step A: Preparation of 2-[(tributylstannyl)methyl]prop-2-en-1-ol

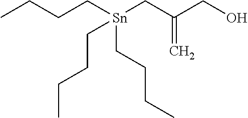

To an oven dried 250 mL round bottom flask was added 10M n-butyllithium (6.1 mL) and the solution set under nitrogen atmosphere. The butyllithium was diluted with ether (34 mL) and the solution cooled to 0° C. using ice/water bath. TMEDA (11.2 mL) and 2-methyl-2-propenyl-1-ol (2 g) was added to the solution via syringe and the resulting mixture stirred for 20 minutes at 0° C. THF (24 mL) was added to the slurry and allowed to warm to room temperature while stirring over night. Tributyltin chloride (8.3 mL) was added to the solution and the resulting solution was stirred for 45 minutes. The solution became cloudy. The solution was quenched with saturated ammonium chloride solution and extracted with ether (100 mL). The organics were then washed with water, brine and then dried over magnesium sulfate. The organics were filtered and evaporated in vacuo. The residue was purified via flash column purification with silica gel eluting with 0-50% ethyl acetate/hexane to afford the title compound. ¹HNMR (500 MHz, CDCl₃) δ: 4.79-4.74 (m, 1H), 4.68-4.64 (m, 1H), 4.00 (dd, J=4.7, 10.9 Hz, 2H), 1.70-1.62 (m, 2H), 1.54-1.45 (m, 6H), 1.40-1.1.29 (m, 10H), 0.96-0.87 (m, 20H).

### Step B: Preparation of (1S)-1-(4-fluorophenyl)-3-[(2S,3R)-2-{4-[2-(hydroxymethyl)prop-2-en-1-yl]phenyl-1-(4-{3-[(methylsulfonyl)amino]propyl}phenyl)-4-oxoazetidin-3-yl]propyl acetate

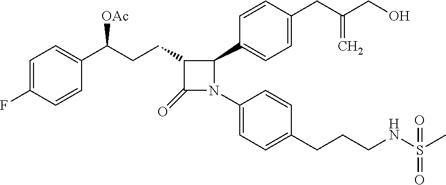

The title compound was prepared using 2-[(tributylstannyl)methyl]prop-2-en-1-ol and (i-10b wherein R¹⁰ is —H) according to the procedure from Example 2, step B. m/z (ES) 645 (M+Na)⁺, 563 (M-OAc)⁺.

### Step C: Preparation of (1S)-3-[(2S,3R)-2-{4-[2,3-dihydroxy-2-(hydroxymethyl)propyl]phenyl-1-(4-{3-[(methylsulfonyl)amino]propyl}phenyl)-4-oxoazetidin-3-yl]1-(4-fluorophenyl)propyl acetate

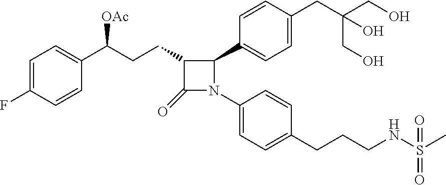

The title compound was prepared from the intermediate of step B according to the procedure from Example 2, step C. m/z (ES) 679 (M+Na)⁺, 597 (M-OAc)⁺.

### Step D: Preparation of N-[3-(4-{(2S,3R)-2-{4-[2,3-dihydroxy-2-(hydroxymethyl)propyl]phenyl}-3-[(3S)-3-(4-fluorophenyl)-3-hydroxypropyl]-4-oxoazetidin-1-yl}phenyl)propyl]methanesulfonamide

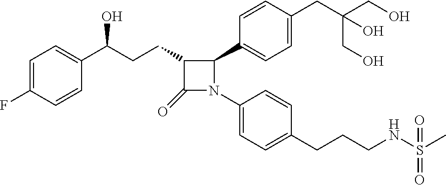

The title compound was prepared from the intermediate of step C according to the procedure from Example 2, step D. m/z (ES) 615 (M+H)⁺, 597 (M-OH)⁺.

### Example 4

### N-[3-(4-[(2S,3R)-2-{4-[4,5-dihydroxy-4-(hydroxymethyl)pentyl]phenyl}-3-[(3S)-3-(4-fluorophenyl)-3-hydroxypropyl]-4-oxoazetidin-1-yl}phenyl)propyl]methanesulfonamide

### Step A: Preparation of 2,2-dimethyl-5-prop-2-yn-1-yl-1,3-dioxan-5-yl acetate

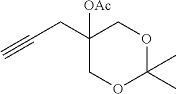

A 100 mL round bottom flask is charged with magnesium turnings (164 mg, 6.73 mmol) and mercury(II)chloride (2.8 mg) and set under nitrogen atmosphere. Ether (1 mL) is added to the solids and the suspension cooled to 0° C. using ice/water bath. Propargyl bromide (1 mL, 6.73 mmol) was added via syringe dropwise maintaining an internal temperature of 5° C. After complete addition, the solution was stirred for 1 hour at 0° C. The cooling bath was removed and the solution warmed to room temperature.

After checking if the gridnard reaction was complete (small acetone quench of an liquot and mass spectra analysis to see Grignard reacted to acetone), 2,2-dimethyl-1,3-dioxan-5-one (500 mg, 3.84 mmol) was added dropwise via syringe and the mixture stirred for two hours. The reaction mixture was quenched with sat. aq. NH₄Cl (25 mL) and then extracted with ethyl acetate (50 mL). The organic layer was dried over Na₂SO₄, filtered and the solvent removed under vacuum to afford the crude intermediate.

The crude intermediate was dissolved in CH₂Cl₂ (10 mL) under nitrogen atmosphere. To the resulting solution was added simultaneously by syringe acetic anhydride (434 μL, 4.60 mmol) and TEA (640 μL, 4.60 mmol). To the reaction mixture was added DMAP (56 mg, 0.46 mmol). The reaction mixture was stirred for 3 hrs at room temperature at which time the reaction was quenched by the addition of 1N aq. HCl (10 mL). The reaction mixture was transferred to a separatory funnel and the organic layer was separated. The organic layer was washed with aq. NaHCO₃ (10 mL), water (5 mL), brine, dried, filtered and the solvent removed under vacuum to afford the title compound which was used without further purification. ¹HNMR (500 MHz, CDCl₃) δ: 4.15 (d, J=12.6 Hz, 2H), 4.03 (J=12.6 Hz, 2H), 2.98 (s, 1H), 2.10 (s, 3H), 1.88 (s, 2H), 1.47 (s, 3H), 1.42 (s, 3H).

### Step B: Preparation of (1S-3-[(2S,3R)-2-(4-{3-[5-(acetyloxy)-2,2-dimethyl-1,3-dioxan-5-yl]prop-1-yn-1-yl}phenyl)-1-(4-{3-[(methylsulfonyl)amino]propyl}phenyl)-4-oxoazetidin-3-yl]-1-(4-fluorophenyl)propyl acetate

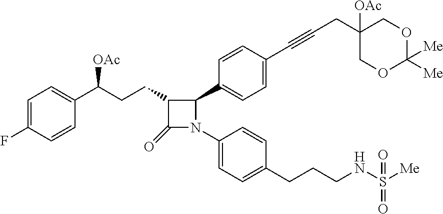

The title compound was prepared from the intermediate of step A and the intermediate (i-10b wherein R¹⁰ is —H) according to the procedure for Example 1, step D. m/z (ES) 703 (M-OAc)⁺.

### Step C: Preparation of (1S)-3-[(2S,3R)-2-(4-{3-[5-(acetyloxy)-2,2-dimethyl-1,3-dioxan-5-yl]propyl}phenyl)-1-(4-{3-[(methylsulfonyl)amino]propyl}phenyl)-4-oxoazetidin-3-yl]-1-(4-fluorophenyl)propyl acetate

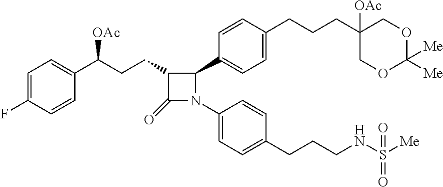

A round bottom flask was charged with 10% Pd—C (50 mg) and 30 mg 20% Pd(OH)₂—C. EtOAc (˜1 mL) was added to cover the solid catalyst mixture. To this mixture was added a solution of the intermediate from Step B, (80 mg, 0.11 mmol) in ethanol (3 mL). The resulting suspension set under hydrogen atmosphere and stirred vigorously for 1 hr. The catalysts were filtered, solids washed with ethanol and the solvent was removed under vacuum to obtain partially hydrogenated intermediate. The reaction procedure was repeated as above. A round bottom flask was charged with 10% Pd—C (50 mg) and 30 mg 20% Pd(OH)₂—C. EtOAc (˜1 mL) was added to cover the solid catalyst mixture. To this mixture was added a solution of the intermediate from above in ethanol (3 mL). The resulting suspension set under hydrogen atmosphere and stirred vigorously for 2 hours. The catalyst was filtered through filter aid and MgSO₄ and washed with EtOH/EtOAc. The filtrate was concentrated in vacuo to afford the title compound which was used without further purification. m/z (ES) 707 (M-OAc)⁺.

### Step D: Preparation of 4-{4-[(2S,3R)-3-[(3S)-3-(acetyloxy)-3-(4-fluorophenyl)propyl]-1-(4-{3-[(methylsulfonyl)amino]propyl}phenyl)-4-oxoazetidin-2-yl]phenyl}-1,1-bis(hydroxymethyl)butyl acetate

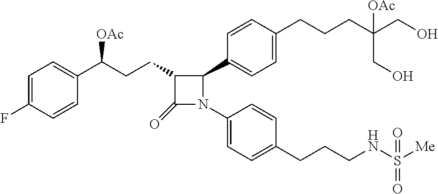

The title compound was prepared from the intermediate of step C according to the procedure for Example 1, step F. m/z (ES) 667 (M-OAc)⁺.

### Step E: Preparation of N-[3-(4-{(2S,3R)-2-{4-[4,5-dihydroxy-4-(hydroxymethyl)pentyl]phenyl}-3-[(3S)-3-(4-fluorophenyl)-3-hydroxypropyl]-4-oxoazetidin-1-yl}phenyl)propyl]methanesulfonamide

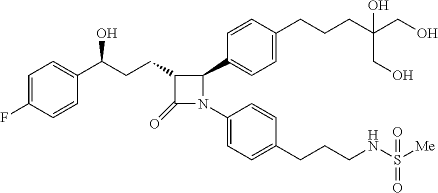

The title compound was prepared from the intermediate of step D according to the procedure for Example 1, step G. m/z (ES) 625 (M-OH)⁺ and 665 (M+Na)⁺¹HNMR (500 MHz, CD₃OD) δ: 7.35-7.31 (m, 2H), 7.27 (d, J=8.1 Hz, 2H), 7.24 (d, J=8.1 Hz, 2H), 7.18 (d, J=8.5 Hz, 2H), 7.10 (d, J=8.5 Hz, 2H), 7.03 (app, t, J=8.6 Hz, 2H), 4.79 (br d, J=2.1 Hz, 1H), 4.60 (br dd, J=5.1, 6.6 Hz, 1H), 3.33-3.31 (m, 4H)), 3.08-3.03 (m, 1H), 3.02 (t, J=6.8 Hz, 2H), 2.89 (s, 3H), 2.64-2.58 (m, 3H), 1.99-1.83 (m, 4H), 1.82-1.74 (m, 2H), 1.74-1.66 (m, 2H), 1.55-1.50 (m, 2H).

### Example 5

### N-[3-(4-{(2S,3R)-2-{4-[5,6-dihydroxy-5-(hydroxymethyl)hexyl]phenyl}-3-[(3S)-3-(4-fluorophenyl)-3-hydroxypropyl]-4-oxoazetidin-1-yl}phenyl)propyl]methanesulfonamide

### Step A: Preparation of (1S)-1-(4-fluorophenyl)-3-[(3R,4S)-1-(4-{3-[(methylsulfonyl)amino]propyl}-phenyl)-2-oxo-4-(4-vinylphenyl)azetidin-3-yl]propyl acetate (i-10c wherein R¹⁰=—H)

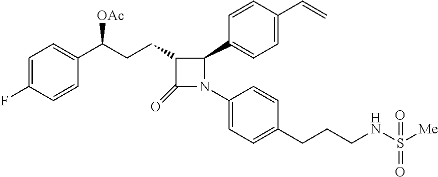

To a solution of (i-10b wherein R¹⁰ is —H and R¹⁸ is vinyl) (200 mg, 0.26 mmol) in anhydrous dioxane (4 mL) was added lithium chloride (33 mg, 0.78 mmol) and dichloro-Bis(triphenylphosphine)palladium (30 mg, 0.03 mmol) and the resulting solution set under nitrogen atmosphere. Vinyl tributyltin (100 μL, 0.312 mmol) was then added to the solution via syringe and the resulting mixture was heated to 90° C. for 4 hours. After cooling to room temperature, the solution was evaporate in vacuo and the residue was dissolved in ethyl acetate (10 mL). The organics were washed with water (5 mL), brine (5 mL), dried over magnesium sulfate, filtered, and evaporated in vacuo. Preparative plate purification eluting with 40% ethyl acetate/60% hexane afforded the title compound. m/z (ES) 519 (M-OAc)⁺ and 579 (M+H)⁺¹HNMR (500 MHz, CDCl₃) δ: 7.44 (d, J=8.2 Hz, 2H), 7.32-7.28 (m, 4H), 7.21 (d, J=8.5 Hz, 2H), 7.07-7.03 (m, 4H), 6.73 (dd, J=11.0, 17.6, 1H), 5.79 (d, J=17.6, 1H), 5.73 (t, J=6.6, 1H), 5.30 (d, J=11.0, 1H), 6.61 (d, J=2.3, 1H), 4.30 (t, J=5.9, 1H), 3.15-3.07 (m, 3H), 2.94 (s, 3H), 2.63 (t, J=7.3, 2H), 2.08-2.03 (m, 5H), 1.92-1.82 (m, 4H).

### Step B: Preparation of 5-but-3-en-1-yl-2,2-dimethyl-1,3-dioxane-5-yl acetate

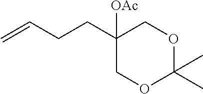

To a dry 100 mL roundbottom flask was charged with a 0.5M solution of ethynylmagnesium bromide in THF (11.5 mL, 5.77 mmol) under nitrogen atmosphere. The resulting solution was cooled to 0° C. in an ice bath. To the cooled solution was added slowly a solution of 2,2-dimethyl-1,3-dioxane-5-one (500 mg, 3.84 mmol) in 5 mL dry THF. The ice bath was removed and the resulting reaction mixture was stirred at ambient temperature for 1.5 hrs. The reaction mixture was quenched with sat. aq. NH₄Cl (5 mL) and then extracted with ethyl acetate (10 mL). The organic layer was dried over Na₂SO₄, filtered and the solvent removed under vacuum to afford the crude intermediate.

The crude intermediate was dissolved in CH₂Cl₂ (10 mL) under nitrogen atmosphere. To the resulting solution was added simultaneously by syringe acetic anhydride (434 μL, 4.60 mmol) and TEA (640 μL, 4.60 mmol). To the reaction mixture was added DMAP (56 mg, 0.46 mmol). The reaction mixture was stirred for 3 hrs at room temperature at which time the reaction was quenched by the addition of 1N aq. HCl (10 mL). The reaction mixture was transferred to separatory funnel and the organic layer was separated. The organic layer was washed with aq. NaHCO₃ (10 mL), water (5 mL), brine, dried, filtered and the solvent removed under vacuum to afford the title compound which was used without further purification. ¹HNMR (500 MHz, CDCl₃) δ: 5.86-5.76 (m, 1H), 5.06 (dd, J=1.4, 17.0 Hz, 1H), 5.00 (dd, J=1.4, 10.3 Hz, 1H), 4.03 (d, J=12.3 Hz, 2H), 3.87 (d, J=12.3 Hz, 2H), 2.09 (s, 3H), 2.08-2.05 (m, 4H), 1.44 (s, 3H), 1.41 (s, 3H).

### Step C: Preparation of (1S)-3-[(2S,3R)-2-(4-{(1E)-4-[5-(acetyloxy)-2,2-dimethyl-1,3-dioxan-5-yl]but-1-en-1-yl}phenyl)-1-(4-{3-[(methylsulfonyl)amino]propyl}phenyl)-4-oxoazetidin-3-yl]-1-(4-fluorophenyl)propyl acetate:

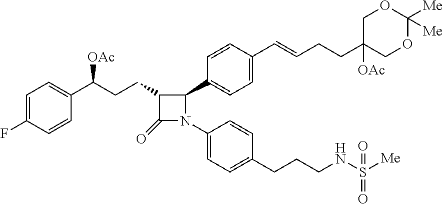

To a solution of the intermediate from Step A (100 mg, 0.17 mmol) and the intermediate from step B (48 mg, 0.20 mmol) in anhydrous dichloromethane (1 mL) under nitrogen atmosphere was added Zhan catalyst (13 mg, 0.20 mmol) and the resulting mixture heated to 40° C. for two hours. The reaction was cooled to room temperature and evaporated in vacuo. Preparative plate purification eluting with 30% ethyl acetate/70% hexane afforded the title compound. m/z (ES) 719 (M-OAc)⁺.

### Step D: Preparation of (1S)-3-[(2S,3R)-2-(4-{4-[5-(acetyloxy)-2,2-dimethyl-1,3-dioxan-5-yl]butyl}phenyl)-1-(4-{3-[(methylsulfonyl)amino]propyl}phenyl)-4-oxoazetidin-3-yl]-1-(4-fluorophenyl)propyl acetate

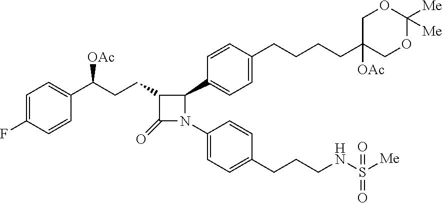

The title compound was prepared from the intermediate of step C according to the procedure for Example 1, step E. m/z (ES) 721 (M-OAc)⁺.

### Step E: Preparation of 5-{4-[(2S,3R)-3-[(3S)-3-(acetyloxy)-3-(4-fluorophenyl)propyl]-1-(4-{3-[(methylsulfonyl)amino]propyl}phenjl)-4-oxoazetidin-2-yl]phenyl}-1,1-bis(hydroxymethyl)pentyl acetate

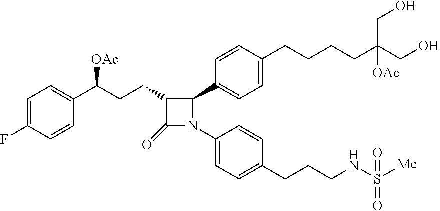

The title compound was prepared from the intermediate of step D according to the procedure for Example 1, step F. m/z (ES) 681 (M-OAc)⁺.

### Step F: Preparation of N-[3-(4-{(2S,3R)-2-{4-[5,6-dihydroxy-5-(hydroxymethyl)hexyl]phenyl}-3-[(3S)-3-(4-fluorophenyl)-3-hydroxypropyl]-4-oxoazetidin-1-yl}phenyl)propyl]methanesulfonamide

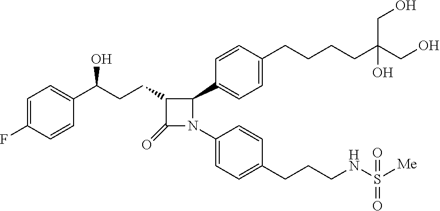

The title compound was prepared from the intermediate of step E according to the procedure for Example 1, step G. m/z (ES) 639 (M-OH)⁺ and 679 (M+Na)⁺¹HNMR (500 MHz, CD₃OD) δ: 7.35-7.31 (m, 2H), 7.27 (d, J=8.1 Hz, 2H), 7.24 (d, J=8.1 Hz, 2H), 7.18 (d, J=8.5 Hz, 2H), 7.10 (d, J=8.5 Hz, 2H), 7.03 (app, t, J=8.6 Hz, 2H), 4.79 (br d, J=2.1 Hz, 1H), 4.60 (br dd, J=5.1, 6.6 Hz, 1H), 3.33-3.31 (m, 4H)), 3.08-3.03 (m, 1H), 3.02 (t, J=6.8 Hz, 2H), 2.89 (s, 3H), 2.64-2.58 (m, 3H), 1.99-1.83 (m, 4H), 1.82-1.74 (m, 2H), 1.74-1.66 (m, 2H), 1.55-1.50 (m, 2H).

### Example 6

### N-{3,[4-((3R,4S)-3-[(3S)-3-(4-fluorophenyl)-3-hydroxpropyl]-2-oxo-4-{4-[1,2,5,6-tetrahydroxy-5-(hydroxoymethyl)hexyl]phenyl}azetidin-1-yl)phenyl]propyl}methanesulfonamide

### Step A: Preparation of (1S-3-[(2S,3R)-2-(4-{4-[5-(acetyloxy)-2,2-dimethyl-1,3-dioxan-5-yl]-1,2-dihydroxybutyl}phenyl)-1-(4-{3-[(methylsulfonyl)amino]propyl}phenyl)-4-oxoazetidin-3-yl]-1-(4-fluorophenyl)propyl acetate

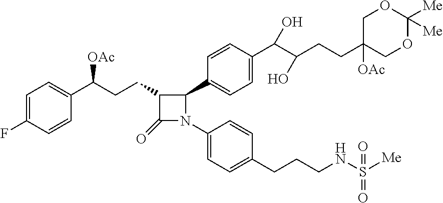

To a solution of (1S)-3-[(2S,3R)-2-(4-{(1E)-4-[5-(acetyloxy)-2,2-dimethyl-1,3-dioxan-5-yl]but-1-en-1-yl}phenyl)-1-(4-{3-[(methylsulfonyl)amino]propyl}phenyl)-4-oxoazetidin-3-yl]-1-(4-fluorophenyl)propyl acetate (see Example 5, Step C) (35 mg, 0.045 mmol) in 8:1 acetone/water (1 mL) was added N-methyl morpholine-N-oxide (9 mg, 0.090 mmol) followed by OsO₄ (2.5% solution in isopropanol, 40 μL, 0.001 mmol) and the resulting mixture stirred for 3 hours at room temperature. The solution was diluted with dichloromethane (10 mL) and washed with 1N HCl (3 mL) followed by water (3 mL) and Brine (3 mL). The organics were dried over magnesium sulfate, filtered and evaporated in vacuo. Preparative plate purification eluting with 5% methanol/95% dichloromethane afforded the title compound. m/z (ES) 835 (M+Na)⁺, 753 (M-OAc)⁺.

### Step B: Preparation of 5-{4-[(2S,3R)-3-[(3S)-3-(acetyloxy)-3-(4-fluorophenyl)propyl]-1-(4-{3-[(methylsulfonyl)amino]propyl}phenyl)-4-oxoazetidin-2-yl]phenyl}-4,5-dihydroxy-1,1-bis(hydroxymethyl)pentyl acetate

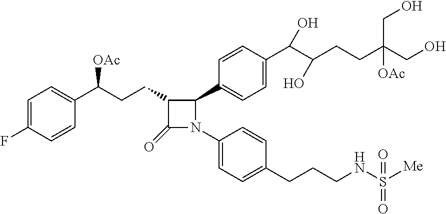

The title compound was prepared from the intermediate of step A according to the procedure for Example 1, step F. m/z (ES) 713 (M-OAc)⁺.

### Step C: Preparation of N-{3,[4-((3R,4S)-3-[(3S)-3-(4-fluorophenyl)-3-hydroxpropyl]-2-oxo-4-{4-[1,2,5,6-tetrahydroxy-5-(hydroxoymethyl)hexyl]phenyl}azetidin-1-yl)phenyl]propyl}methanesulfonamide

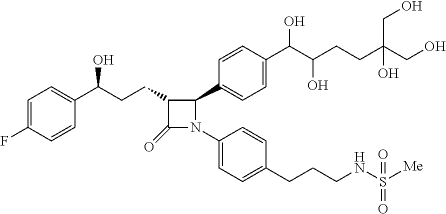

The title compound was prepared from the intermediate of step B according to the procedure for Example 1, step G. m/z (ES) 711 (M+Na)⁺, 671 (M-OH)⁺.

### Example 7

### N-[3-(4-{(2S,3R)-2-{4-[3,4-dihydroxy-3-(hydroxymethyl)butyl]phenyl}-3-[3-(4-fluorophenyl)-3-oxopropyl]-4-oxoazetidin-1-yl}phenyl)propyl]methanesulfonamide

### Step A: Preparation of N-{3-[4-((2S,3R)-3-[(3S)-3-(4-fluorophenyl)-3-hydroxypropyl]-2-{4-[2-(5-hydroxy-2,2-dimethyl-1,3-dioxane-5-yl)ethyl]phenyl}-4-oxoazetidine-1-yl)phenyl]propyl}methanesulfonamide

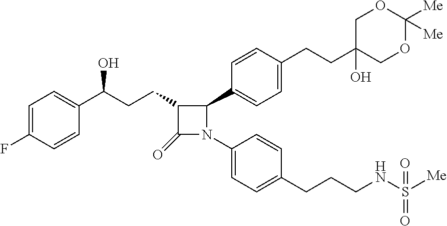

The title compound was prepared from the intermediate (1S)-3-[(2S,3R)-2-(4-{2-[5-(acetyloxy)-2,2-dimethyl-1,3-dioxan-5-yl]ethyl}phenyl)-1-(4-{3-[(methylsulfonyl)amino]propyl}phenyl)-4-oxoazetidin-3-yl]-1-(4-fluorophenyl)propyl acetate (see Ex. 1, Step E) (100 mg, 0.133 mmol) according to the procedure for Example 1, step G. m/z (ES) 651 (M-OH)⁺.

### Step B: Preparation of N-{3-[4-((2S,3R)-3-[3-(4-fluorophenyl)-3-oxopropyl]-2-{4-[2-(5-hydroxy-2,2-dimethyl-1,3-dioxane-5-yl)ethyl]phenyl}-4-oxoazetidine-1-yl)phenyl]propyl}methanesulfonamide

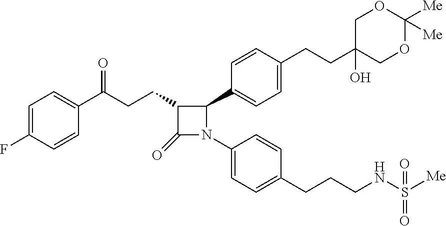

To a solution of the intermediate in Step A (71 mg, 0.12 mmol) in dichloromethane (1 mL) was added via syringe Dess-Martin reagent (15% solution in CH₂Cl₂, 690 μL, 0.24 mmol) and the resulting solution stirred for 2 hours at room temperature. Preparative plate purification eluting with 90% ethyl acetate/10% hexane afforded the title compound. m/z (ES) 667 (M+H)⁺.

### Step C: Preparation of N-[3-(4-{(2S,3R)-2-{4-[3,4-dihydroxy-3-(hydroxymethyl)butyl]phenyl}-3-[3-(4-fluorophenyl)-3-oxopropyl]-4-oxoazetidin-1-yl}phenyl)propyl]methanesulfonamide

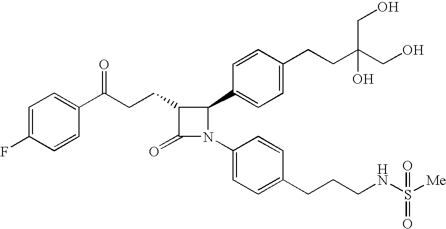

The title compound was prepared from the intermediate of step B according to the procedure for Example 1, step F. m/z (ES) 627 (M+H)⁺. ¹HNMR (500 MHz, CDCl₃) δ: 8.02 (dd, J=5.3, 8.7 Hz, 2H), 7.28 (d, J=8.3 Hz, 2H), 7.22 (dd, J=4.1, 8.1 Hz, 4H), 7.158 (app t, J=8.7 Hz, 2H), 7.07 (d, J=8.5 Hz, 2H), 4.72 (br d, J=2.2 Hz, 1H), 4.23 (br t, J=6.6 Hz, 1H), 3.73 (br d, J=10.5 Hz, 2H), 3.65 (br d, J=10.5 Hz, 2H), 3.35-3.27 (m, 1H), 3.22-3.15 (m, 2H), 3.13 (dd, J=6.6, 13.3 Hz, 2H), 2.94 (s, 3H), 2.74-2.69 (m, 2H), 2.63 (t, J=7.4 Hz, 2H), 2.43 (hept, 7.1 Hz, 1H), 2.34-2.22 (m, 3H), 1.86 (pent, 7.1 Hz, 2H), 1.80-1.75 (m, 2H).

### Example 8

### (2S,3S,4S,5R,6R)-6-[4-{4-[(2S,3R)-3-[(3S)-3-(4-fluorophenyl)-3-hydroxypropyl]-1-(4-{3-[(methylsulfonyl)amino]propyl}phenyl)-4-oxoazetidin-2-yl]pheny;}-2-hydroxy-2-(hydroxymethyl)butoxy]-3,4,5-trihydroxytetrahydro-2H-pyran-2-carboxylic acid

### Step A: Preparation of methyl (2S,3S,4S,5R,6R)-3,4,5-tris(acetyloxy)-6-[2-(acetyloxy)-4-{4-[(2S,3R)-3-[(3S)-3-(acetyloxy)-3-(4-fluorophenyl)propyl]-1-(4-{3-[(methylsulfonyl)amino]propyl}phenyl)-4-oxoazetidin-2-yl]phenyl}-2-(hydroxymethyl)butoxy]tetrahydro-2H-pyran-2-carboxylate

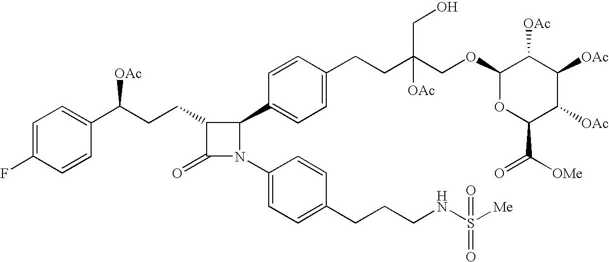

To a solution of 3-{4-[(2S,3R)-3-[(3S)-3-(acetyloxy)-3-(4-fluorophenyl)propyl]-1-(4-{3-[(methylsulfonyl)amino]propyl}phenyl)-4-oxoazetidin-2-yl]phenyl}-1,1-bis(hydroxymethyl)propyl acetate (prep Example 1, Step F; 100 mg, 0.140 mmol) and 2,3,4-tri-O-Acetyl-α-D-glucuronic acid methyl ester, trichloroacetimidate (70 mg, 0.145 mmol) in dichloromethane (3 mL) set under nitrogen atmosphere was added BF₃-etherate (4 μL, 0.028 mmol) and the resulting solution stirred for two hours at room temperature. Additional BF₃-etherate (4 μL, 0.028 mmol) was added and the mixture stirred overnight at room temperature. The mixture was washed with water (2 mL); then the organics separated, dried over magnesium sulfate, filtered and concentrated in vacuo. Preparative plate purification eluting with 5% MeOH/95% dichloromethane afforded the title compound. m/z (ES) 653 (M-OAc and glucoside)⁺, 970 (M-OAc)⁺, and 1051 (M+Na)⁺

### Step B: Preparation of methyl (2S,3S,4S,5R,6R)-6-[4-{4-[(2S,3R)-3-[(3S)-3-(4-fluorophenyl)-3-hydroxypropyl]-1-(4-{3-[(methylsulfonyl)amino]propyl}phenyl)-4-oxoazetidin-2-yl]phenyl}-2-hydroxy-2-(hydroxymethyl)butoxy]-3,4,5-trihydroxytetrahydro-2H-pyran-2-carboxylate

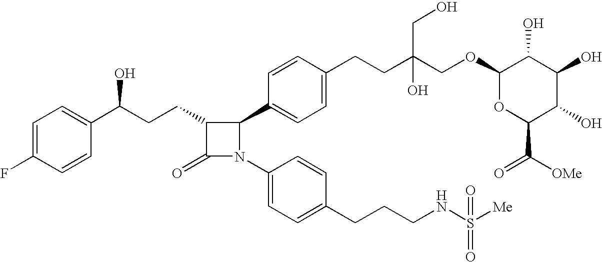

The title compound was prepared from the intermediate of step A (36 mg, 0.035 mmol) according to the procedure for Example 1, step G. m/z (ES) 611 (M-OH and glucoside)⁺ and 841 (M+Na)⁺

### Step C: Preparation of (2S,3S,4S,5R,6R)-6-[4-{4-[(2S,3R)-3-[(3S)-3-(4-fluorophenyl)-3-hydroxypropyl]-1-(4-{3-[(methylsulfonyl)amino]propyl}phenyl)-4-oxoazetidin-2-yl]pheny;}-2-hydroxy-2-(hydroxymethyl)butoxy]-3,4,5-trihydroxytetrahydro-2H-pyran-2-carboxylic acid

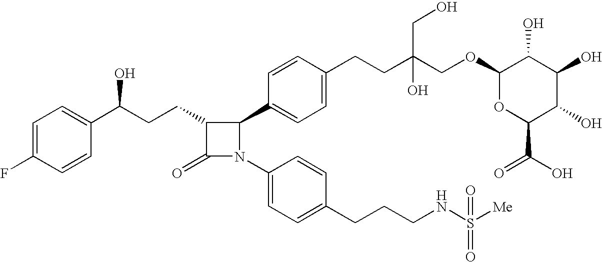

To a solution of the intermediate from step B (7.0 mg, 0.086 mmol) in methanol/water (4:1) was added triethylamine (120 μL, 0.086 mmol) and the resulting solution stirred for 3 hours at room temperature. Purification by Mass-directed HPLC with gradient eluant of 0-100% acetonitrile in water (0.1% TFA buffered) afforded the title compound. (m/z (ES) 611 (M-OH and glucoside)⁺ and 827 (M+Na)⁺¹HNMR (500 MHz, CD₃OD) δ: 7.38-7.30 (m, 2H), 7.28-7.22 (m, 4H), 7.18 (d, J=8.4 Hz, 2H), 7.11 (d, J=8.4 Hz, 2H), 7.03 (app, t, J=8.5 Hz, 2H), 4.80 (br d, J=2.0 Hz, 1H), 4.60 (br dd, J=5.2, 6.6 Hz, 1H), 4.38 (d, J=8.6 Hz, 1H) 3.92-3.85 (m, 1H) 3.75 (s, 2H), 3.71 (s, 2H), 3.60-3.50 (m, 3H), 3.09-3.02 (m, 1H), 3.01 (t, J=6.9 Hz, 2H), 2.89 (s, 3H), 2.75-2.67 (m, 2H), 2.62 (t, 7.5 Hz, 2H), 1.97-1.82 (m, 3H), 1.81-1.73 (m, 3H).

### Example 9

### (2S,3S,4S,5R,6R)-6-{[(1S)-3-[(2S,3R)-2-{4-[3,4-dihydroxy-3-(hydroxymethyl)butyl]phenyl}-1-(4-{3-[(methylsulfonyl)amino]propyl}phenyl)-4-oxoazetidin-3-yl]-1-(4-fluorophenyl)propyl]oxy}-3,4,5-trihydroxytetrahydro-2H-pyran-2-carboxylic acid

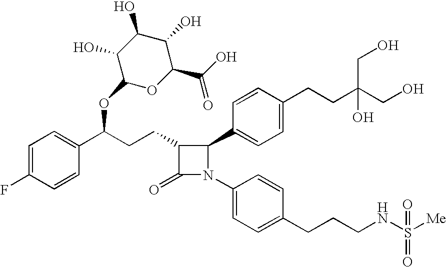

¹HNMR (500 MHz, CDCl₃) δ: 7.40 (dd, J=5.3, 8.7 Hz, 2H), 7.27 (d, J=8.3 Hz, 2H), 7.24 (d, J=8.1 Hz, 2H), 7.18 (d, J=8.5 Hz, 2H), 7.09 (d, J=8.5 Hz, 2H), 7.03 (app, t, J=8.6 Hz, 2H), 5.01 (br dd, J=5.2, 6.6 Hz, 1H), 4.78 (br d, J=2.1 Hz, 1H), 3.99 (d, J=8.6 Hz, 1H) 3.53 (s, 4H), 3.07-3.04 (m, 1H), 3.00 (t, J=6.8 Hz, 2H), 2.87 (s, 3H), 2.72-2.65 (m, 2H), 2.59 (t, 7.6 Hz, 2H), 1.97-1.83 (m, 3H), 1.80-1.74 (m, 3H).

### Example 10

### (3R,4S-4-{4-[3,4-dihydroxy-3-(hydroxymethyl)butyl]phenyl}-3-[(3S)-3-(4-fluorophenyl)-3-hydroxypropyl]1-[4-(3-hydroxypropyl)phenyl]azetidin-2-one

### Step A: Preparation of (1S)-1-(4-fluorophenyl)-3-[(3R,4S)-1-(4-iodophenyl)-2-oxo-4-(4-{[(trifluoromethyl)sulfonyl]oxy}phenyl)azetidin-3-yl]propyl acetate

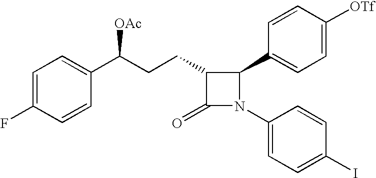

The phenol 1-7a (500 mg; 0.894 mmol) was dissolved in CH₂Cl₂ (5 ml) and triethylamine (143 μl) and N-phenyltrifluoromethane sulfonimide (350 mg; 0.983 mmol) were added and stirred together at room temperature for 2 h. The volatiles from the reaction mixture were removed under reduced pressure and the residues partitioned with 2N-hydrochloric acid (50 ml) and diethyl ether (2×50 ml). The ethereal extracts were combined and dried over anhydrous MgSO4 powder, filtered and concentrated to afford crude product which was purified by silica gel preparative plates eluted with EtOAc and hexanes (1:1) to afford the title compound. m/z (ES) 714 (M+Na)⁺, 632 (M-OAc)⁺.

### Step B: Preparation of (1S)-3-[(3R,4S)-1-[4-(3,3-diethoxyprop-1-yn-1-yl)phenyl]-2-oxo-4-(4-{[(trifluoromethyl)sulfonyl]oxy}phenyl)azetidin-3-yl]-1-(4-fluorophenyl)propyl acetate

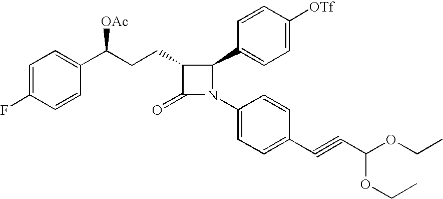

A solution of the iodotriflate (210 mg; 0.304 mmol) from step A, propiolaldehyde diethyl acetal (97.3 mg; 0.759 mmol), tetrakis(triphenylphosphine)palladium(0) (17.4 mg; 0.015 mmol), copper(I) iodide (2.9 mg; 0.015 mmol) and triethylamine (1 ml) were dissolved in dry CH₂Cl₂. Nitrogen gas bubbled slowly through the solution for approximately 5 minutes, the reaction vessel sealed under a nitrogen atmosphere and stirred at room temperature for 4 h. The volatiles were removed from the reaction products under reduced pressure. The product was purified by silica gel preparative plates and eluted with CH₂Cl₂ and MeOH (98:2 v/v) to afford the title compound. m/z (ES) 714 (M+Na)⁺, 632 (M-OAc)⁺.

### Step C: Preparation of (1S)-3-{(2S,3R)-2-(4-{[5-(acetyloxy)-2,2-dimethyl-1,3-dioxan-5-yl]ethynyl}phenyl)-1-[4-(3,3-diethoxyprop-1-yn-1-yl)phenyl]-4-oxoazetidin-3-yl}-1-(4-fluorophenyl)propyl acetate

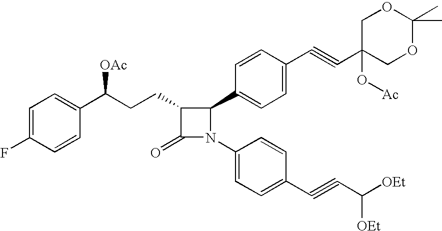

The triflate from step B (750 mg; 1.084 mmol) and 5-ethynyl-2,2-dimethyl-1,3-dioxan-5-yl acetate (i-6) (236 mg; 1.193 mmol), tetrabutyl ammonium iodide (20 mg; 0.054 mmol), tetrakis(triphenylphosphine)palladium(0) (62.6 mg; 0.054 mmol), copper(I) iodide (10.3 mg; 0.054 mmol) were dissolved in DMF (2.5 ml) and triethylamine (2.5 ml). Nitrogen gas was slowly bubbled through the solution for 5 minutes then the reaction vessel was sealed under a nitrogen atmosphere and the contents heated in a bath set at 70° C. for 4 h. The reaction mixture was concentrated under reduced pressure to remove the volatiles. Purification of the resulting product by silica gel preparative plates eluted with dichloromethane and MeOH (97:3 v/v) afforded the title compound. m/z (ES) 680 (M-OAc)⁺.

### Step D: Preparation of (1S)-3-{(2S,3R)-2-(4-{2-[5-(acetyloxy)-2,2-dimethyl-1,3-dioxan-5-yl]ethyl}phenyl)-1-[4-(3,3-diethoxypropyl)phenyl]-4-oxoazetidin-3-yl}-1-(4-fluorophenyl)propyl acetate

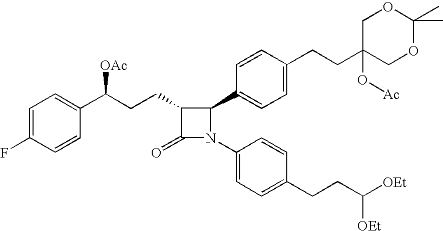

The bis-acetylene compound (300 mg) from Step C was dissolved in ethanol (15 ml) and 20% palladium hydroxide on carbon (50 mg) was added to the ethanol solution. After three vacuum then flush with hydrogen cycles, the ethanol solution was hydrogenated at atmospheric pressure and at room temperature with hydrogen gas contained in a balloon reservoir for 3 h when the reaction was judged to be essentially over by lc-ms. The spent hydrogenation catalyst was removed by filtering through a 0.45-micron Acrodisk syringe filter and the filtrates obtained were concentrated down. Purification of the product was effected by silica gel preparative plates eluted with dichloromethane and MeOH (97:3 v/v) to afford the title compound. m/z (ES) 770 (M+Na)⁺, 688 (M-OAc)⁺.

### Step E: Preparation of (1S)-3-{(2S,3R)-2-{4-[3,4-dihydroxy-3-(hydroxymethyl)butyl]phenyl}-1-[4-(3,3-dihydroxypropyl)phenyl]-4-oxoazetidin-3-yl}-1-(4-fluorophenyl)propyl acetate

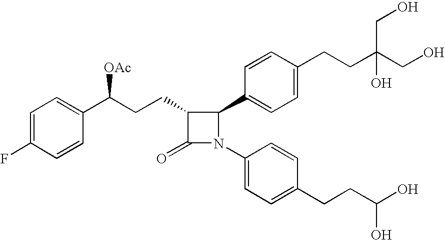

The diethyl acetal (100 mg) from step D was dissolved in THF (1 ml) and 20% TFA in water (1 ml) added. More THF was added using a Pasteur pipette in order to keep the solution homogeneous. The reaction was stirred at room temperature for 22 h and then the volatiles were evaporated using a rotary evaporator with a warm water bath and azeotroped with toluene three times to remove water. The crude reaction product thus obtained was dissolved in MeCN and the resulting solution filtered and the filtrates purified by reverse phase preparative lc-ms collecting on m/z=532. The appropriate fractions containing the product were combined and freeze dried under vacuum to afford the title compound. m/z (ES) 592 (MH⁺)⁺, 532 (M-OAc)⁺.

### Step F: Preparation of (1S)-3-{(2S,3R)-2-{4-[3,4-dihydroxy-3-(hydroxymethyl)butyl]phenyl}-1-[4-(3-hydroxypropyl)phenvyl]-4-oxoazetidin-3-yl}-1-(4-fluorophenyl)propyl acetate

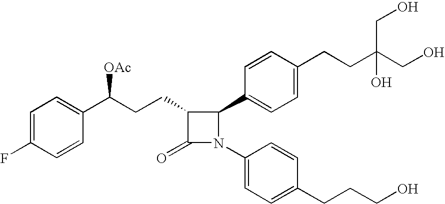

The aldehyde hydrate (10 mg) obtained in step E was dissolved in ethanol (1 ml) and powdered sodium borohydride was added to the alcohol solution. More sodium borohydride was added until 3 mg total of reducing agent was added. The progress of the reaction was monitored by analytical lc-ms. Dilute 2N hydrochloric acid was added from a Pasteur pipette to quench the reaction and the aqueous ethanol solution adjusted to around pH=5 to pH paper. A small amount of water was then added and the crude reaction mixture was partitioned with two 5 ml portions of CH₂Cl₂ and then two 5 ml portions of EtOAc. The combined organic extracts were dried over anhydrous sodium sulfate, filtered and the filtrates obtained concentrated under reduced pressure. The crude product was dissolved in MeCN (2 ml) filtered and this solution purified from the other side products by prep ic-ms collecting on m/z=533.2. The aqueous acetonitrile product fractions from the purification were concentrated down under reduced pressure to give the desired compound. m/z (ES) 616 (M+Na)⁺, 534 (M-OAc)⁺.

### Step G: Preparation of (3R,4S)-4-{4-[3,4-dihydroxy-3-(hydroxymethyl)butyl]phenyl}-3-[(3S)-3-(4-fluorophenyl)-3-hydroxypropyl]-1-[4-(3-hydroxypropyl)phenyl]azetidin-2-one

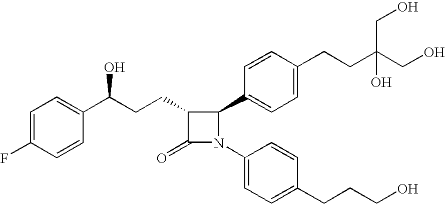

The acetate from step F above (3 mg) was dissolved in ethanol (1 ml) along with potassium cyanide (0.5 mg) and the reaction mixture warmed in a heating bath set at 50° C. for approximately 0.75 h. The reaction was cooled to room temperature and the solution purified by reverse phase preparative lc-ms collecting on m/z=533. The aqueous acetonitrile product fractions were concentrated down under reduced pressure to give the desired compound. m/z (ES) 574 (M+Na)⁺, 534 (M-OH)⁺. ¹H-NMR (400 MHz, CD₃OD) δ: 7.24-7.35 (complex, 6H), 7.18 (d, J=8 Hz, 2H), 7.10 (d, J=8 Hz, 2H), 7.04 (t, J=8 Hz, 2H), 4.80 (br s, 1H), 4.61 (br app t, J=5.5 Hz, 1H), 3.53 (br s, 4H), 3.52 (t, J=6 Hz, 2H); 3.08 (m, 1H), 2.71 (t, J=8.5 Hz, 2H), 2.60 (t, 7.6 Hz, 2H), 1.98-1.84 (m, 3H), 1.81-1.75 (m, 3H).

### Example 11

### 3-(4-{(2S,3R)-2-{4-[3,4-dihydroxy-3-(hydroxymethyl)butyl]phenyl}-3-[(3S)-3-(4-fluorophenyl)-3-hydroxypropyl]-4-oxoazetidin-1-yl}phenyl)propanoic acid

### Step A: Preparation of 4-(4-{(2S,3R)-3-[(35)-3-(acetyloxy)-3-(4-fluorophenyl)propyl]-1-[4-(3,3-dihydroxypropyl)phenyl]-4-oxoazetidin-2-yl}phenyl)-2-hydroxy-2-(hydroxymethyl)butyl acetate

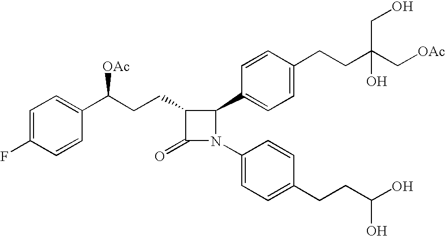

(1S)-3-{(2S,3R)-2-(4-{2-[5-(acetyloxy)-2,2-dimethyl-1,3-dioxan-5-yl]ethyl}phenyl)-1-[4-(3,3-diethoxypropyl)phenyl]-4-oxoazetidin-3-yl}-1-(4-fluorophenyl)propyl acetate (see Example 10 Step D) the diethyl acetal (100 mg) was dissolved in THF (2 ml) and a 20% TFA in water (2 ml) added. More THF was added using a Pasteur pipette in order to keep the solution homogeneous. The reaction was stirred at room temperature for 16 h and then the volatiles were evaporated using a rotary evaporator with a warm water bath. The crude reaction product thus obtained was dissolved in MeCN and the resulting solution filtered and the filtrates purified by reverse phase preparative lc-ms collecting on m/z=634.2 and 573.2. The aqueous acetonitrile product fractions were concentrated down under reduced pressure to give the desired compound. m/z (ES) 657 (M+Na)⁺, 573 (M-OAc)⁺.

### Step B: Preparation of 3-[4-((2S,3R)-3-[(3S)-3-(acetyloxy)-3-(4-fluorophenyl)propyl]-2-{4-[4-(acetyloxy)-3-hydroxy-3-(hydroxymethyl)butyl]phenyl}-4-oxoazetidin-1-yl)phenyl]propanoic acid

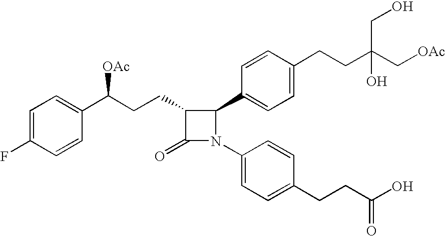

Aqueous solutions of sulfamic acid (3.5 mg in 0.5 ml of water) and sodium chlorite (3.5 mg in 0.5 ml of water) were prepared around 15 minutes in advance of running the oxidation reaction. A solution of the product from Step A (7.5 mg; 0.0015 mmol) in THF was prepared and stirred at room temperature. The aqueous solution of sulfamic acid was added to the aldehyde hydrate THF solution, followed by the solution of sodium chlorite and the color of the reaction solution became pale yellow. After approximately 20 minutes a small aliquot of the reaction was removed and analyzed by lc-ms to determine that the starting material had been consumed and the product had formed. The reaction was treated with aqueous sodium sulfite solution to destroy excess oxidant then the material in the reaction vessel was extracted with CH₂Cl₂ (2×5 ml) and EtOAc (2×5 ml). The organic extracts were combined dried with Na₂SO₄ powder, filtered and concentrated under reduced pressure. The residues from evaporation were triturated with EtOH (5 ml) and the ethanol solution filtered through a 0.45 micron Acrodisk syringe filter. The solution was concentrated down and used in the acetate deprotection step described in Step C below.

### Step C: Preparation of 3-(4-{(2S,3R)-2-{4-[3,4-dihydroxy-3-(hydroxymethyl)butyl]phenyl}-3-[(3S)-3-(4-fluorophenyl)-3-hydroxypropyl]-4-oxoazetidin-1-yl}phenyl)propanoic acid

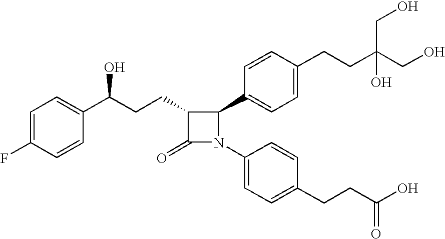

The crude material from Step B (˜3 mg) was dissolved in ethanol (1.5 ml) and potassium cyanide (1.5 mg) powder was added to the ethanol solution. The reaction was monitiored by lc-ms. After 4 h the reaction was judged to be complete. The reaction solution was filtered through a 0.45 micron Acrodisk syringe filter and the solution thus obtained purified by reverse phase preparative lc-ms collecting on m/z=548.2. The aqueous acetonitrile product fractions from the preparative lc-ms were concentrated down under reduced pressure to give the desired compound. m/z (ES) 588 (M+Na)⁺; 548 (M-OH)⁺. ¹H-NMR (400 MHz, CD₃OD) δ: 7.20-7.31 (complex, 6H), 7.16 (d, J=8.5 Hz, 2H) 7.09 (d, J=8.5 Hz, 2H), 7.01 (t, J=8.5 Hz, 2H), 4.77 (d, J=2 Hz, 1H), 4.58 (t, J=5 Hz, 1H), 3.51 (br s, 4H), 3.03 (br, 1H), 2.80 (t, J=8 Hz, 2H), 2.68 (complex, 2H), 2.51 (t, 8 Hz, 2H), 1.78-1.95 (complex, 4H), 1.74 (complex, 2H).

### Example 12

### 3-(4-{(2S,3R)-2-{4-[3,4-dihydroxy-3-(hydroxymethyl)butyl]phenyl}-3-[3-(4-fluorophenyl)-3-oxopropyl]-4-oxoazetidin-1-yl}phenyl)propanoic acid

### Step A: Preparation of (3R,4S)-1-[4-(3,3-diethoxypropyl)phenyl]-3-[(3S)-3-(4-fluorophenyl)-3-hydroxypropyl]-4-{4-[2-(5-hydroxy-2,2-dimethyl-1,3-dioxan-5-yl)ethyl]phenyl}azetidin-2-one

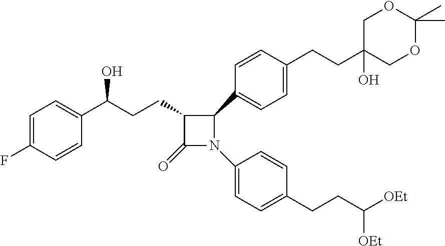

(1S)-3-{(2S,3R)-2-(4-{2-[5-(acetyloxy)-2,2-dimethyl-1,3-dioxan-5-yl]ethyl}phenyl)-1-[4-(3,3-diethoxypropyl)phenyl]-4-oxoazetidin-3-1}-yl-(4-fluorophenyl)propyl acetate from (see Example 10 Step D), the diacetate (34 mg; 0.045 mmol) was dissolved in methanol and potassium cyanide (3 mg) was added and the resulting solution stirred for 6 h in a heating bath set at 50° C. By that time the starting material had essentially been consumed as judged by lc-ms. The reaction mixture was evaporated under reduced pressure to remove the methanol and the product that remained purified by silica gel preparative plate eluted with dichloromethane:methanol (95:5 v/v) to afford the title compound. m/z (ES) 686 (M+Na)⁺; 646 (M-OH)⁺

### Step B: Preparation of (3R,4S)-1-[4-(3,3-diethoxypropyl)phenyl]-3-[3-(4-fluorophenyl)-3-oxopropyl]-4-{4-[2-(5-hydroxy-2,2-dimethyl-1,3-dioxan-5-yl)ethyl]phenyl}azetidin-2-one

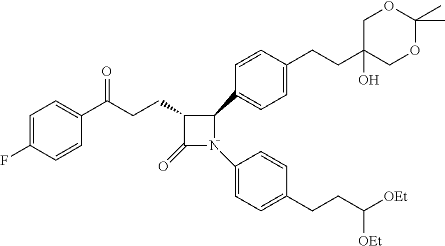

The diol (16 mg; 0.024 mmol) from Step A above was dissolved in CH₂Cl₂ and the Dess-Martin periodinate oxidant (13.3 mg; 0.031 mmol) was added to the solution and the resulting reaction mixture stirred at room temperature. After 1 h the starting material had been consumed. A few drops of saturated aqueous sodium sulfate solution were added to the reaction stirred for 5 minutes to destroy the excess oxidant. The reaction was partitioned between water and CH₂Cl₂ (3×5 ml) and the combined methylene chloride extracts were dried over anhydrous Na₂SO₄ powder, filtered and concentrated under reduced pressure. The product that remained after evaporation was purified on a preparative silica gel plate eluted with CH₂Cl₂ and methanol (95:5 v/v) to afford the title compound. m/z (ES) 684 (M+Na)⁺; 644 (M-OH)⁺

### Step C: Preparation of (3R,4S-4-{4-[3,4-dihydroxy-3-(hydroxymethyl)butyl]phenyl}-1-[4-(3,3-dihydroxypropyl)phenyl]-3-[3-(4-fluorophenyl)-3-oxopropyl]azetidin-2-one

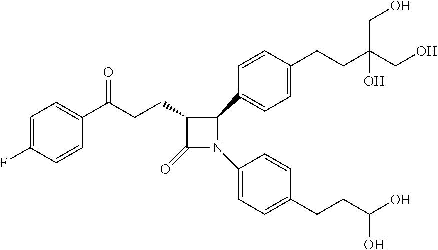

The ketone (8 mg) from Step C of this example was dissolved in THF (0.5 ml) and 0.25 ml of a 20% aqueous TFA solution was added. More THF was added from a Pasteur pipette to keep the reaction mixture homogeneous. The reaction was warmed in a heating bath set at 50° C. for approximately 2.5 h then the volatiles were removed under reduced pressure and the residues azeotrophed with toluene (3×). The crude product was purified by reverse phase lc-ms collecting on m/z=548.2. The aqueous acetonitrile product fractions from the preparative lc-ms were concentrated down under reduced pressure to give the desired compound. m/z (ES) 570 (M+Na)⁺; 530 (M-OH)⁺

### Step D: Preparation of 3-(4-{(2S,3R)-2-{4-[3,4-dihydroxy-3-(hydroxymethyl)butyl]phenyl}-3-[3-(4-fluorophenyl)-3-oxopropyl]-4-oxoazetidin-1-yl}phenyl)propanoic acid

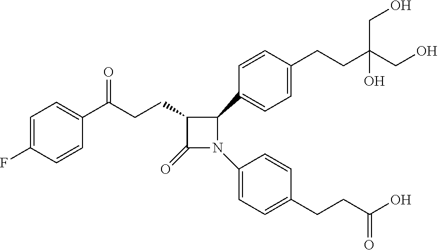

Aqueous solutions of sulfamic acid (2.5 mg in 0.5 ml of water) and sodium chlorite (2.5 mg in 0.5 ml of water) were prepared around 15 minutes in advance of running the oxidation reaction. A solution of the product from Step C (5 mg; 0.009 mmol) in THF was prepared and stirred at room temperature. The aqueous solution of sulfamic acid followed was added to the aldehyde hydrate THF solution, followed by the solution of sodium chlorite. After approximately 15 minutes a small aliquot of the reaction was removed and analyzed by lc-ms to determine that the starting material had been consumed and the product had formed. The reaction was treated with a few drops of aqueous sodium sulfite solution to destroy the excess oxidant, then the material in the reaction vessel was extracted with CH₂Cl₂ (3×2 ml) and EtOAc (3×2 ml) and diethyl ether (3×2 ml). The organic extracts were combined dried with Na₂SO₄ powder, filtered and concentrated under reduced pressure. The residues from evaporation were triturated with EtOH (ml) and the ethanol solution filtered through a 0.45 micron Acrodisk syringe filter and purified by reverse phase preparative lc-ms collecting m/z=546.2. The aqueous acetonitrile product fractions from the preparative lc-ms were concentrated down under reduced pressure to give the desired compound. m/z (ES) 586 (M+Na)⁺; 545 (M-OH)⁺. ¹H-NMR (400 MHz, CD₃OD) δ: 8.05 (complex, 2H), 7.05-7.30 (complex, 10H), 4.86 (br s, 1H), 3.51 (s, 4H), 3.25 (m, 2H), 3.15 (m, 1H), 2.67 (complex, 2H), 2.60 (t, 7 Hz, 2H), 2.30 (q, J=7.5 Hz, 2H), 1.81-1.74 (complex, 4H).

### Example 13

### (3R,4S)-4-{4-[3,4-dihydroxy-3-(hydroxymethyl)butyl]phenyl}-3-[(3S)-3-(4-fluorophenyl)-3-oxopropyl]-1-[4-(3-hydroxypropyl)phenyl]azetidin-2-one

### Step A: Preparation of 4-[(2S,3R)-3-[(35)-3-(4-fluorophenyl)-3-hydroxypropyl]-2-(4-iodophenyl)-4-oxoazetidin-1-yl]phenyl trifluoromethanesulfonate

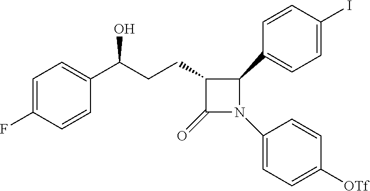

(3R,4S)-3-[(3S)-3-(4-Fluorophenyl)-3-hydroxypropyl]-1-(4-hydroxyphenyl)-4-(4-iodophenyl)azetidin-2-one (2 g; 3.866 mmol) was dissolved in CH₂Cl₂ (40 ml) and then N-phenyltrifluoromethane sulfonimide (1.45 g; 4.059 mmol) followed by triethylamine (593 μl; 3.658 mmol) were added and the reaction mixture stirred for 2 h. The formation of the desired product was monitored by analytical lc-ms over time. The methylene chloride reaction solvent was evaporated off under reduced pressure and the residues obtained were partitioned between 1N hydrochloric acid (25 ml) and diethyl ether (3×25 ml). The ethereal extracts were combined, washed with 2N sodium hydroxide (10 ml) and brine (50 ml). The ethereal solution was dried over anhydrous MgSO4 powder, filtered, and the filtrates evaporated under reduced pressure to leave a yellow oil. Purification of the oil on preparative silica gel plates eluted with EtOAc:Hexanes (3:2 v/v) led to a recovery of the desired triflate compound. m/z (ES) 672 (M+Na)⁺; 650 (MH⁺)⁺; 632 (M-OH)⁺

### Step B: Preparation of 4-[(2S,3R)-3-[3-(4-fluorophenyl)-3-oxopropyl]-2-(4-iodophenyl)-4-oxoazetidin-1-yl]phenyl trifluoromethanesulfonate

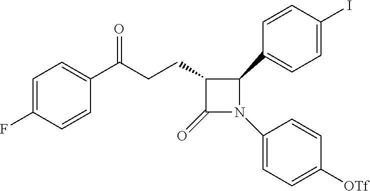

The alcohol from Step A (500 mg; 0.770 mmol) was dissolved in anhydrous CH₂Cl₂ (7.5 ml) to which the Dess-Martin periodinate (408.2 mg; 0.962 mmol) was added in small batches over 3 minutes and the resulting reaction mixture stirred at room temperature for 3 h. The reaction mixture was quenched with saturated aqueous sodium sulfite solution (5 ml), stirred for 5 minutes and then partitioned with water (50 ml) and diethyl ether (3×50 ml). The ethereal extracts were combined, washed with brine and dried over anhydrous MgSO4 powder then filtered. The filtrates obtained were concentrated to leave a yellow oil. Separation by preparative tlc on silica gel plates afforded the title compound. m/z (ES) 670 (M+Na)⁺; 648 (MH⁺)⁺

### Step C: Preparation of 5-({4-[(2S,3R)-3-[3-(4-fluorophenyl)-3-oxopropyl]-4-oxo-1-(4-{[(trifluoromethyl)sulfonyl]oxy}phenyl)azetidin-2-yl]phenyl}ethynyl)-2,2-dimethyl-1,3-dioxan-5-yl acetate

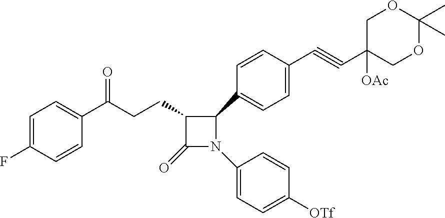

The ketone (350 mg; 0.541 mmol) from Step B and 5-ethynyl-2,2-dimethyl-1,3-dioxan-5-yl acetate (i-6) (161 mg; 0.811 mmol) from above, tetrakis(triphenylphosphine)palladium(0) (31.2 mg; 0.027 mmol), copper(I) iodide (5.1 mg; 0.027 mmol) were dissolved in CH2Cl2 (2 ml) and triethylamine (2 ml). Nitrogen gas bubbled slowly through the solution for approximately 5 minutes. The reaction mixture was sealed under a nitrogen atmosphere in the flask and stirred at room temperature for 4 h. The volatiles from the crude reaction mixture were then removed from the reaction products under reduced pressure to leave a thick yellow oil residue. Purification by preparative tlc on silica gel plates and elution with EtOAc and Hexanes (1:1 v/v) of this afforded the desired acetylene coupling product. m/z (ES) 658 (M-OAc)⁺.

### Step D: Preparation of 5-[(4-{(2S,3R)-1-{4-[3-(benzyloxy)prop-1-vn-1-yl]phenyl}-3-[3-(4-fluorophenyl)-3-oxopropyl]-4-oxoazetidin-2-yl}phenyl)ethynyl]-2,2-dimethyl-1,3-dioxan-5-yl acetate

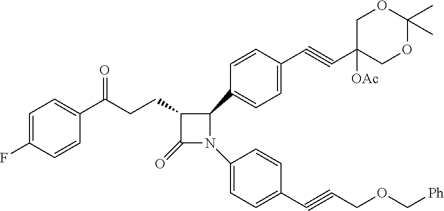

The triflate (300 mg; 0.418 mmol) and benzyl propargyl ether (244 mg; 1.672 mmol) tetrabutylammonium iodide (7.7 mg; 0.021 mmol), tetrakis(triphenylphosphine)-palladium(0) (24.2 mg; 0.021 mmol), copper(I) iodide (4 mg; 0.021 mmol) were dissolved in a mixture of DMF (1.5 ml) and triethylamine (1.5 ml). Nitrogen gas was slowly bubbled through the solution for 3 minutes then the reaction mixture was sealed under a nitrogen atmosphere and the contents heated in a bath set at 70° C. for 5 h. The reaction mixture was concentrated under reduced pressure to remove the volatiles. After evaporation a black oil remained. Purification of the black oil by preparative tlc on silica gel plates eluted with EtOAc and Hexanes (1:1 v/v) afforded the title compound.

m/z (ES) 736 (M+Na)⁺; 654 (M-OAc)⁺.

### Step E: Preparation of 5-[2-(4-{(2S,3R)-3-[3-(4-fluorophenyl)-3-oxopropyl]-1-[4-(3-hydroxypropyl)phenyl]-4-oxoazetidin-2-yl}phenyl)ethyl]-2,2-dimethyl-1,3-dioxan-5-yl acetate

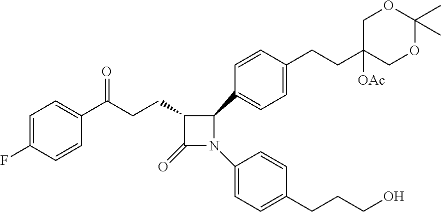

The bis-acetylene compound (75 mg) from Step D was dissolved in ethanol (15 ml) and 20% palladium hydroxide on carbon (5 mg) and 10% palladium on carbon (15 mg) was added to the ethanol solution. After three vacuum then flush with hydrogen cycles, the ethanol solution was hydrogenated at atmospheric pressure and at room temperature with hydrogen gas contained in a balloon reservoir for 4 h when the reaction was judged to be essentially over by lc-ms. The spent hydrogenation catalyst was removed by filtering through a 0.45-micron Acrodisk syringe filter and the filtrates obtained concentrated down to leave a yellow colored oil. Purification of the oil was effected by reverse phase lc-ms. The recovery from the purification of the desired hydrogenated product (collected on m/z-624.2) was 18 mg along with 25 mg the over-reduced product, 5-[2-(4-{(2S,3R)-3-[3-(4-fluorophenyl)-3-hydroxypropyl]-1-[4-(3-hydroxypropyl)phenyl]-4-oxoazetidin-2-yl}phenyl)ethyl]-2,2-dimethyl-1,3-dioxan-5-yl acetate, (collected on m/z=626.2) as an epimeric mixture. m/z (ES) 573 (M-OAc)⁺.

### Step F: Preparation of (3R,4S)-4-{4-[3,4-dihydroxy-3-(hydroxymethyl)butyl]phenyl}-3-[3-(4-fluorophenyl)-3-oxopropyl]-1-[4-(3-hydroxypropyl)phenyl]azetidin-2-one

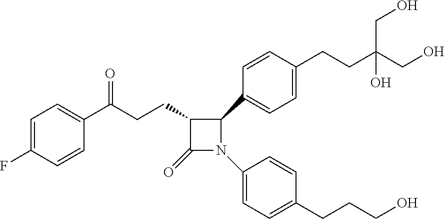

The ketone product (15 mg) from Step E of this example was dissolved in THF (0.5 ml) and 0.25 ml of a 20% aqueous TFA solution was added. A few drops more of THF was added from a Pasteur pipette to make the reaction mixture homogeneous. The reaction was warmed in a heating bath set at 50° C. for approximately 2.5 h then the volatiles were removed under reduced pressure and the residues azeotrophed with toluene (3×). The crude product was purified by reverse phase lc-ms collecting the fractions that had an m/z=591.2 and 531.2. The product containing fractions were concentrated to leave 8.6 mg of products. This 8.6 mg of material was dissolved in ethanol (0.5 ml) and stirred with potassium trimethylsilanoate (2.5 mg) at room temperature for 45 minutes when the reaction was judged to be complete by analytical lc-ms. The alcohol solution was filtered and the filtrates thus obtained purified by preparative reverse phase lc-ms collecting on m/z=549.2 and 531.2. The aqueous acetonitrile product fractions from the preparative lc-ms were concentrated down under reduced pressure to give the desired compound. m/z (ES) 572 (M+Na)⁺; 532 (M-OH)⁺. ¹H-NMR (400 MHz, CD₃OD) δ: 8.03 (complex, 2H), 7.27 (d, J=8 Hz, 2H), 7.19 (complex, 6H),), 7.09 (d, J=8 Hz, 2H), 4.88 (br d, J=2 Hz, 1H), 3.51 (br s, 4H), 3.25 (m, 2H), 3.15 (dt, J=7.5, 2 Hz), 2.67 (complex, 2H), 2.59 (t, J=7 Hz, 2H), 2.27 (q, 7.5 Hz, 2H), 1.69-1.80 (complex, 4H).

### Example 14

### (3R,4S)-4-{4-[3,4-dihydroxy-3-(hydroymethyl)butyl]phenyl}-3-[(3S)-3-(4-fluorophenyl)-3-hydroxypropyl]-1-{4,[4-(methylsulfonyl)butyl]phenyl}azetidin-2-one

### Step A: Preparation of 2-[(acetyloxy)methyl]-2-hydroxybut-3-yn-1-yl acetate

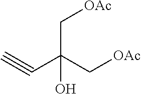

To a cooled solution (0° C.) of 1,3-diacetoxyacetone (17.5 g, 100.0 mmol) in 50 mL dry THF under nitrogen atmosphere was added slowly via syringe a 0.5M solution of ethynylmagnesium bromide in THF (200 mL, 100.0 mmol) and the resulting mixture was stirred for 10 minutes. The ice bath was then removed and the resulting reaction mixture was stirred at ambient temperature for 2.5 hrs. The reaction mixture was quenched with sat. aq. NH₄Cl (100 mL) and then extracted with ethyl acetate (200 mL). The extracts were dried over magnesium sulfate, filtered, and concentrated in vacuo. The residue was purified via Horizon MPLC eluting with a gradient eluant of 0-20% ethyl acetate in hexane to afford the title compound. ¹H-NMR (400 MHz, CDCl₃) δ: 4.28 (d, J=11.5 Hz, 2H), 4.22 (J=11.5 Hz, 2H), 3.26 (s, 1H), 2.55 (s, 1H), 2.13 (s, 6H).

### Step B: Preparation of (1S)-3-[(2S,3R)-2-(4-{4-(acetyloxy)-3-[(acetyloxy)methyl]-3-hydroxybut-1-yn-1-yl}phenyl)-4-oxo-1-(4-{[(trifluoromethyl)sulfonyl]oxy}phenyl)azetidin-3-yl]-1-(4-fluorophenyl)propyl acetate

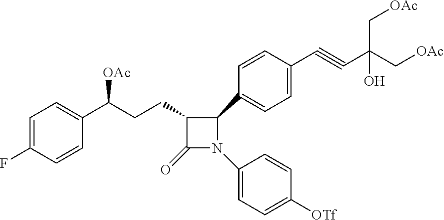

To a solution of 4-[(2S,3R)-3-[(3S)-3-(4-fluorophenyl)-3-hydroxypropyl]-2-(4-iodophenyl)-4-oxoazetidin-1-yl]phenyl trifluoromethanesulfonate (2 g, 3.08 mmol; see Example 13, Step A) in CH₂Cl₂ (25 mL) under nitrogen atmosphere was added acetic anhydride (0.4 mL, 4.30 mmol), triethylamine (0.75 mL, 5.38 mmol) and DMAP. The reaction mixture was stirred at RT for 1 hr and the solvent removed under vacuum. The residue was purified by MPLC (silica column) with stepwise gradient elution; (0-100% EtOAc/hexanes as eluent) to afford the acetate intermediate (i-14).

To a solution of 2-[(acetyloxy)methyl]-2-hydroxybut-3-yn-1-yl acetate (5.2 g; 26.0 mmol) and the acetate intermediate i-14 (10.5 grams, 15.2 mmol) in DMF (150 ml) under nitrogen atmosphere was added dichlorobis(triphenylphosphine)palladium(0) (1.10 g; 1.5 mmol), copper(I) iodide (580 mg; 3.0 mmol) and triethylamine (14.8 ml, 106.4 mmol) and the resulting solution stirred at room temperature for 3 h. The reaction was cooled and then quenched with 1N HCl (150 mL) and diluted with 300 mL ethyl acetate. The organic was separated and washed with water (100 mL) and brine (100 mL). The organics were then dried over magnesium sulfate, filtered and concentrated in vacuo. Horizon MPLC purification eluting with a gradient system of 0-50% ethyl acetate/hexane afforded the title compound. m/z (ES) 704 (M-OAc)⁺.

### Step C: Preparation of (1S)-3-((2S,3R)-2-(4-[4-(acetyloxy)-3-[(acetyloxy)methyl]-3-hydroxybut-1-yn-1-yl}phenyl)-1-{4-[4-(methylsulfonyl)but-1-yn-1-yl]phenyl}-4-oxoazetidin-3-yl)-1-(4-fluorophenyl)propyl acetate

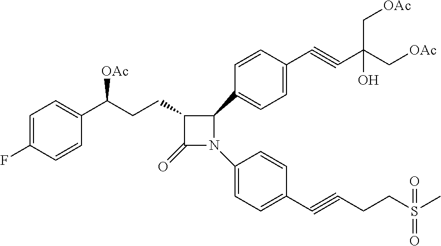

The triflate from Step B (110 mg; 0.14 mmol) and 4-(methylsulfonyl)but-1-yne (i-13) (28 mg; 0.21 mmol) tetrabutylammonium iodide (52 mg; 0.14 mmol), tetrakis(triphenylphosphine)-palladium(0) (106 mg; 0.09 mmol), and copper(I) iodide (9 mg; 0.04 mmol) were dissolved in a mixture of DMF (3 mL) and triethylamine (0.137 mL). Nitrogen gas was slowly bubbled through the solution for 3 minutes then the reaction mixture was sealed under a nitrogen atmosphere and the contents heated in a bath set at 70° C. for 3 h. The reaction mixture was concentrated under reduced pressure to remove the volatiles. Purification of the resulting product by preparative silica gel plates eluted with EtOAc and Hexanes (1:1 v/v) afforded on isolation the title compound. m/z (ES) 746 (M+H)⁺.

### Step D: Preparation of (1S)-3-((2S,3R)-2-(4-[4-(acetyloxy)-3-[(acetyloxy)methyl]-3-hydroxybutyl}phenyl)-1-{4-[4-(methylsulfonyl)butyl]phenyl}-4-oxoazetidin-3-yl)-1-(4-fluorophenyl)propyl acetate

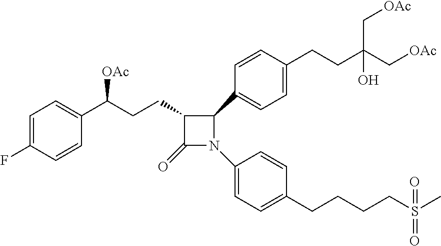

The title compound was prepared from the product of step C according to the procedure from Example 1, step E. m/z (ES) 754 (M+H)⁺.

### Step E: Preparation of (3R,4S)-4-{4-[3,4-dihydroxy-3-(hydroxymethyl)butyl]phenyl}-3-[(3S)-3-(4-fluorophenyl)-3-hydroxypropyl]-1-{4-[4-(methylsulfonyl)butyl]phenyl}azetidin-2-one

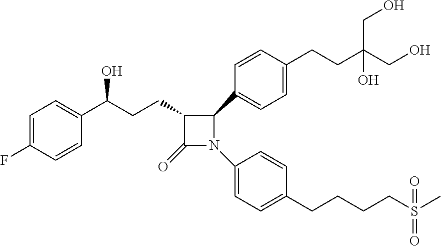

The title compound was prepared from the product of step D according to the procedure from Example 1, step G. m/z (ES) 629 (M+H)⁺.

### Example 15

### (3R,4S)-4-{4-[3,4-dihydroxy-3-(hydroymethyl)butyl]phenyl}-3-[(3S)-3-(4-fluorophenyl)-3-hydroxypropyl]-1-{4,[6-(methylsulfonyl)hexyl]phenyl}azetidin-2-one

### Step A: Preparation of (16)-3-((2S,3R)-2-(4-[4-(acetyloxy)-3-[(acetyloxy)methyl]-3-hydroxybut-1-yn-1-yl}phenyl)-1-{4-[6-(methylsulfonyl)hex-1-yn-1-yl]phenyl}-4-oxoazetidin-3-yl)-1-(4-fluorophenyl)propyl acetate

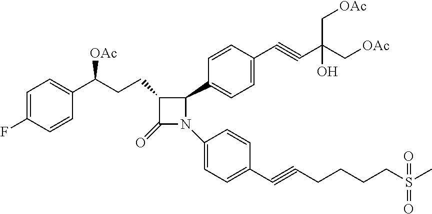

The triflate from Example 14, Step B, (150 mg; 0.20 mmol) and 6-(methylsulfonyl)hex-1-yne (i-13c) (64 mg; 0.40 mmol) tetrabutylammonium iodide (74 mg; 0.20 mmol), tetrakis(triphenylphosphine)-palladium(0) (145 mg; 0.16 mmol), and copper(I) iodide (13 mg; 0.06 mmol) were dissolved in a mixture of DMF (3 mL) and triethylamine (0.20 mL). Nitrogen gas was slowly bubbled through the solution for 3 minutes then the reaction mixture was sealed under a nitrogen atmosphere and the contents heated in a bath set at 70° C. for 3 h. The reaction mixture was concentrated under reduced pressure to remove the volatiles. Purification of the resulting product by preparative silica gel plates eluted with EtOAc and Hexanes (1:1 v/v) afforded on isolation the title compound. m/z (ES) 774 (M+H)⁺.

### Step B: Preparation of (1S)-3-((2S,3R)-2-(4-[4-(acetyloxy)-3-[(acetyloxy)methyl]-3-hydroxybutyl}phenyl)-1-{4-[6-(methylsulfonyl)hexyl]phenyl}-4-oxoazetidin-3-yl)-1-(4-fluorophenyl)propyl acetate

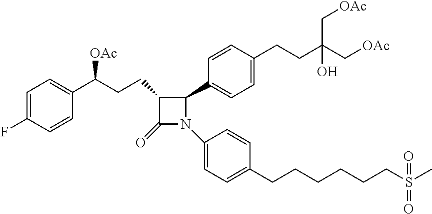

The title compound was prepared from the product of step A according to the procedure for Example 1, step E. m/z (ES) 782 (M+H)⁺.

### Step C: Preparation of (3R,46)-4-{4-[3,4-dihydroxy-3-(hydroxymethyl)butyl]phenyl}-3-[(3S)-3-(4-fluorophenyl)-3-hydroxypropyl]-1-{4-[6-(methylsulfonyl)hexyl]phenyl}azetidin-2-one

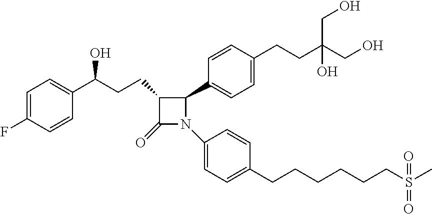

The title compound was prepared from the product of step B according to the procedure for Example 1, step G. m/z (ES) 656 (M+H)⁺.

### Example 16

### Step A: Preparation of (1S)-3-[(2S,3R)-2-(4-{4-(acetyloxy)-3-[(acetyloxy)methyl}-3-hydroxybutyl}phenyl)-4-oxo-1-(4-{[(trifluoromethyl)sulfonyl]oxy}phenyl)azetidin-3-yl]-1-(4-fluorophenyl)propyl acetate

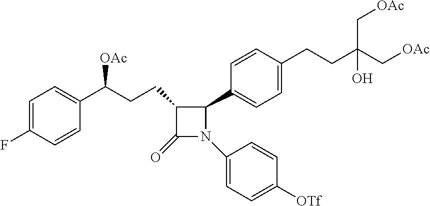

A round bottom flask was charged with 10% Pd—C (500 mg) and EtOAc (˜2 mL) was added to cover the catalyst. To this suspension was added a solution of the intermediate from Example 14, Step B, (2.0, 2.6 mmol) in ethyl acetate (40 mL) and the resulting suspension set under hydrogen atmosphere and stirred vigorously for 1 hr. The catalysts were filtered, solids washed with ethyl acetate and the solvent was removed under vacuum to obtain partially hydrogenated intermediate. The reaction procedure was repeated again as above. The catalyst was filtered through filter aid and MgSO₄ and washed with EtOH/EtOAc. The filtrate was concentrated in vacuo and purified via Horizon MPLC eluting with a linear gradient eluant of 10-70% ethyl acetate in hexane afford the title compound. m/z (ES) 709 (M-OAc)⁺.

### Step B: Preparation of (1S)-3-[(2S,3R)-2-(4-{4-(acetyloxy)-3-[(acetyloxy)methyl}-3-hydroxybutyl}phenyl)-4-oxo-1-(4-vinylphenyl)azetidin-3-yl]-1-(4-fluorophenyl)propyl acetate

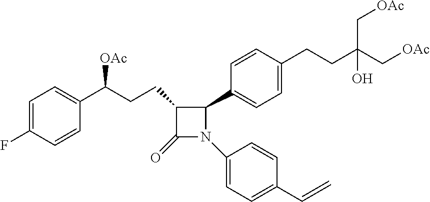

To a solution of the intermediate from Step A (200 mg, 0.26 mmol) in anhydrous dioxane (4 mL) was added lithium chloride (33 mg, 0.78 mmol) and dichloro-Bis(triphenylphosphine)palladium (30 mg, 0.03 mmol) and the resulting solution was set under nitrogen atmosphere. Vinyl tributyltin (100 μL, 0.312 mmol) was then added to the solution via syringe and the resulting mixture was heated to 90° C. for 4 hours. After cooling to room temperature, the solution was evaporate in vacuo and the residue was dissolved in ethyl acetate (10 mL). The organics were washed with water (5 mL), brine (5 mL), dried over magnesium sulfate, filtered, and evaporated in vacuo. Preparative plate purification eluting with 60% ethyl acetate/40% hexane afforded the title compound. m/z (ES) 586 (M-OAc)⁺ and 646 (M+H)⁺

### Step C: Preparation of (1S-3-[(2S,3R)-2-(4-{4-(acetyloxy)-3-[(acetyloxy)methyl}-3-hydroxybutyl}phenyl)-1-[4-(1,2-dihydroxyethyl)phenyl]-4-oxoazetidin-3-yl}-1-(4-fluorophenyl)propyl acetate

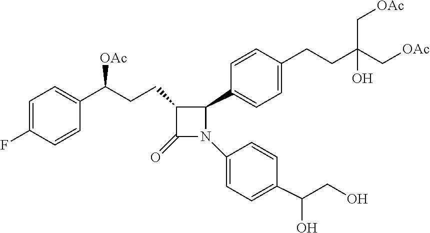

To a solution of the intermediate from Step B (40 mg, 0.06 mmol) in an 8:1 solution of acetone/water was added N-methylmorpholine-N-oxide (14 mg, 0.12 mmol) followed by a 2.5% solution of OsO₄ in isopropanol (60 μL, 0.0006 mmol) and the resulting mixture stirred at room temperature for 3 hours. The mixture was diluted with dichloromethane (10 mL) and washed with 1N HCl (5 mL) followed by Brine (5 mL). The organics were dried over magnesium sulfate, filtered, and concentrated under vacuum. Preparative plate purification eluting with 100% ethyl acetate afforded the title compound. m/z (ES) 620 (M-OAc)⁺, and 680 (M+H)⁺

### Step D: Preparation of (3R,4S)-1-[4-(1,2-dihydroxyethyl)phenyl]-4-{4-[3,4-dihydroxy-3-(hydroxymethyl)butyl]phenyl}-3-[(3S)-3-(4-fluorophenyl)-3-hydroxypropyl}azetidin-2-one

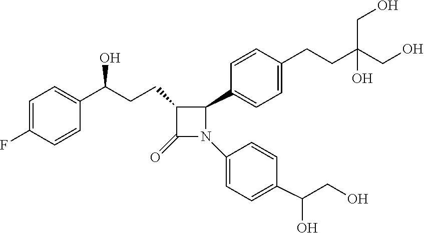

The title compound was prepared from the intermediate of step C according to the procedure for Example 1, step G. m/z (ES) 554 (M+H)⁺, and 536 (MH-H₂O)⁺.

### Example 17

### Step A: Preparation of (1S)-3-[(2S,3R)-2-(4-{4-(acetyloxy)-3-[(acetyloxy)methyl]-3-hydroxybutyl}phenyl)-4-oxo-1-(4-ethylphenyl)azetidin-3-yl]-1-(4-fluorophenyl)propyl acetate

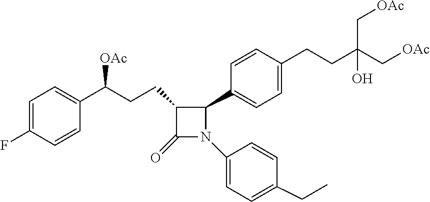

The title compound was prepared from (1S)-3-[(2S,3R)-2-(4-{4-(acetyloxy)-3-[(acetyloxy)methyl}-3-hydroxybutyl}phenyl)-4-oxo-1-(4-vinylphenyl)azetidin-3-yl]-1-(4-fluorophenyl)propyl acetate (Example 16, step B) according to the procedure for Example 1, step E. m/z (ES) 554 (M+H)⁺, and 536 (MH-H₂O)⁺

### Step B

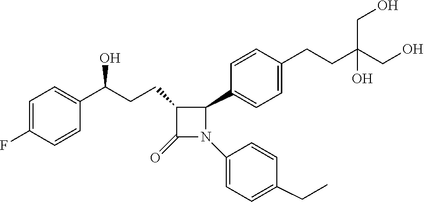

The final product was prepared from the intermediate of step A according to the procedure for Example 1, step G. m/z (ES) 522 (M+H)⁺, and 504 (MH-H₂O)⁺.

Employing procedures similar to those described in Example 16, the following compounds in Table 4 were prepared from their appropriate starting materials:

TABLE 4

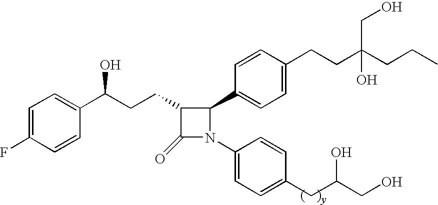

| Example | | |
|---|---|---|
| # | y | m/z (ES) |
| 18 | 1 | 568 (M + H)⁺, 550 (MH—H₂O)⁺ |
| 19 | 2 | 582 (M + H)⁺, 564 (MH—H₂O)⁺ |

Employing procedures similar to those described in Example 17, the following compounds in Table 5 were prepared from their appropriate starting materials:

TABLE 5

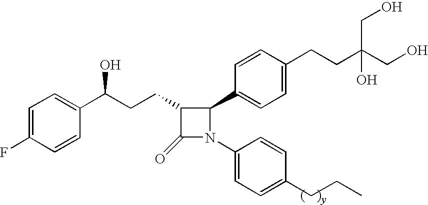

| Example | | |
|---|---|---|
| # | y | m/z (ES) |
| 20 | 1 | 536 (M + H)⁺, 518 (MH—H₂O)⁺ |
| 21 | 2 | 550 (M + H)⁺, 532 (MH—H₂O)⁺ |

### Example 22

### (3R,4S)-3-[(3S)-3-(4-fluorophenyl)-3-hydroxypropyl]-1,4-bis[4-(3-hydroxypropyl)phenyl]azetidin-2-one

### Step A: Preparation of (1S)-3-((2S,3R)-1,2-bis{4-[3-(benzyloxy)prop-1-yn-1-yl]phenyl}-4-oxoazetidin-3-yl)-1-(4-fluorophenyl)propyl acetate

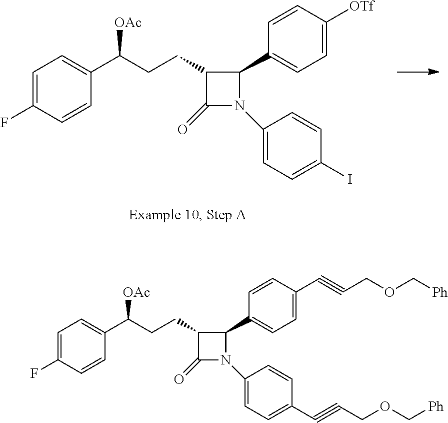

The title compound was prepared from the product of Example 10, Step A and benzyl propargyl ether according to the procedure for Example 1, step D. m/z (ES) 706.1 (M+Na)⁺, 646.2 (M-OAc)⁺.

### Step B: Preparation of (1S)-3-{(2S,3R)-1,2-bis[4-(3-hydroxypropyl)phenyl]-4-oxoazetidin-3-yl}-1-(4-fluorophenyl)propyl acetate

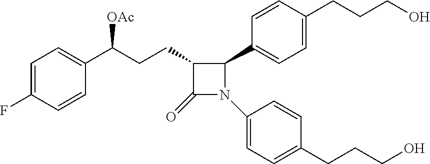

(1S)-3-((2S,3R)-1,2-bis{4-[3-(benzyloxy)prop-1-yn-1-yl]phenyl}-4-oxoazetidin-3-yl)-1-(4-fluorophenyl)propyl acetate (20 mg) was dissolved in ethanol (3 ml) and ethyl acetate (3 ml) and 10% palladium on carbon (7 mg) was added to the solution. After three vacuum then flush with hydrogen cycles, the ethanol solution was hydrogenated at atmospheric pressure and at room temperature with hydrogen gas contained in a balloon reservoir. After 6.5 hours of hydrogenation, the reaction was judged to be essentially over by lc-ms. The spent hydrogenation catalyst was removed by filtering through a 0.45-micron Acrodisk syringe filter and the filtrates obtained concentrated down. This crude product was taken on to the next step. m/z (ES) 556.3 (M+Na)⁺, 496.3 (M-OAc)⁺.

### Step C: Preparation of (3R,4S)-3-[(3S)-3-(4-fluorophenyl)-3-hydroxypropyl]-1,4-bis[4-(3-hydroxypropyl)phenyl]azetidin-2-one

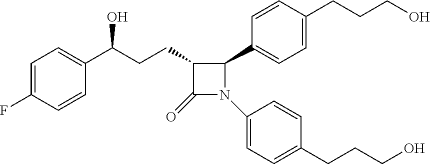

(1S)-3-{(2S,3R)-1,2-bis[4-(3-hydroxypropyl)phenyl]-4-oxoazetidin-3-yl}-1-(4-fluorophenyl)propyl acetate, from Step B (5 mg; 0.0089 mmol) was dissolved in ethanol (0.5 ml) and potassium trimethylsilanoate (1.7 mg; 0.0134 mmol) added and the reaction mixture stirred at room temperature for about 8 hours. The reaction mixture was purified by reversed phase preparative lc-ms collecting on m/z=514.3. The aqueous acetonitrile product fractions containing the desired product were concentrated down to afford the title compound.

¹H-NMR (400 MHz, CD₃OD) δ: 1.72-2.10 (complex, 8H), 2.60 (t, J=8 Hz, 2H), 2.69 (t, J=8 Hz, 2H), 3.06 (broad m, 1H), 3.52 (t, J=6.5 Hz, 2H), 3.56 (t, J=6.5 Hz, 2H), 4.61 (broad t, J=6 Hz, 1H), 4.81 (d, J=1.5 Hz, 1H), 7.00-7.35 (complex, 12H). m/z (ES) 514.3 (M+Na)⁺.

### Example 23

### (3R,4S)-3-[(3S)-3-(4-fluorophenyl)-3-hydroxypropyl]-1,4-bis[4-(4-hydroxybutyl)phenyl]azetidin-2-one

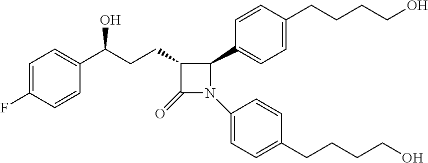

The title compound was prepared from the appropriate starting materials using the procedures described in Examples 22. m/z (ES) 542.3 (M+Na)⁺.

### Example 24

### (3R,4S)-4-{4-[3,4-dihydroxy-3-(hydroxymethyl)butyl]phenyl}-3-[(3S)-3-(4-fluorophenyl)-3-hydroxypropyl]-1-{4-[5-hydroxy-4-(hydroxymethyl)pentyl]phenyl}azetidin-2-one Step A: Preparation of 2-Prop-2-yn-1-ylpropane-1,3-diol

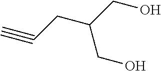

A solution of dimethyl prop-2-yn-1-ylmalonate (1 g; 5.877 mmol) in THF (5 ml) was added gradually to a stirred suspension of lithium aluminum hydride (LAH) (560 mg) in dry THF (5 ml) at 50° C. under a nitrogen atmosphere. The reaction mixture was stirred 24 h and then a 1M solution of LAH in THF (6 ml) added and the reaction stirred at 50° C. for a further 6.5 hours. The reaction mixture was set aside to cool to ambient temperature and then the excess LAH destroyed by the addition of a saturated aqueous Na₂SO₄ solution. Celite® and sodium sulfate were both added to thicken the slurry into moist solid lumps. The lumps were transferred into a Buchner funnel and the product washed off the solid with diethyl ether. The ethereal filtrate was concentrated down under reduced pressure on a rotary evaporator to afford the crude product. The crude diol was purified by preparative tlc on silica gel plates eluted with CH₂Cl₂/MeOH (9:1 v/v) to afford the title compound. ¹H-NMR (400 MHz, CDCl₃) δ: 2.00 (m., 1H), 2.02 (t, J=2.5 Hz, 1H), 2.34 (dd, J=2.5 & 7 Hz, 2H), 3.83 (doublet of ABq, J=11 & 6 Hz, 4H).

### Step B: Preparation of (1S)-1-(4-fluorophenyl)-3-[(3R,4S)-1-{4-[5-hydroxy-4-(hydroxymethyl)pent-1-yn-1-yl]phenyl}-2-oxo-4-(4-{[(trifluoromethyl)sulfonyl]oxy}phenyl)azetidin-3-yl]propyl acetate

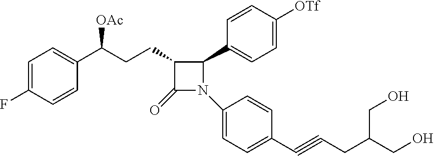

The title compound was prepared from (1S)-1-(4-fluorophenyl)-3-[(3R,4S)-1-(4-iodophenyl)-2-oxo-4-(4-{[(trifluoromethyl)sulfonyl]oxy}phenyl)azetidin-3-yl]propyl acetate (see Example 10, Step A) and 2-prop-2-yn-1-ylpropane-1,3-diol according to the procedure for Example 1, step A. m/z (ES) 700.2 (M+Na)⁺, 618.3 (M-OAc)⁺.

### Step C: Preparation of (1S)-3-((2S,3R)-2-(4-{[5-(acetyloxy)-2,2-dimethyl-1,3-dioxan-5-yl]ethynyl}phenyl)-1-{4-[5-hydroxy-4-(hydroxymethyl)pent-1-yn-1-yl]phenyl}-4-oxoazetidin-3-yl)-1-(4-fluorophenyl)propyl acetate

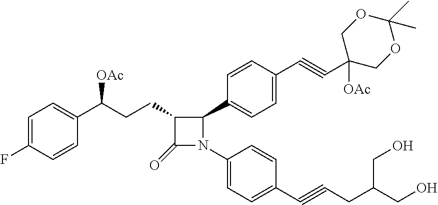

The title compound was prepared from (1S)-1-(4-fluorophenyl)-3-[(3R,4S)-1-{4-[5-hydroxy-4-(hydroxymethyl)pent-1-yn-1-yl]phenyl}-2-oxo-4-(4-{[(trifluoromethyl)sulfonyl]oxy}phenyl)azetidin-3-yl]propyl acetate (from Step B) and 5-ethynyl-2,2-dimethyl-1,3-dioxan-5-yl acetate (i-6) according to the procedure for Example 1, step D. m/z (ES) 666.3 (M-OAc)⁺.

### Step D: Preparation of (1S)-3-((2S,3R)-2-(4-{2-[5-(acetyloxy)-2,2-dimethyl-1,3-dioxan-5-yl]ethyl}phenyl)-1-{4-[5-hydroxy-4-(hydroxymethyl)pentyl]phenyl}-4-oxoazetidin-3-yl)-1-(4-fluorophenyl)propyl acetate

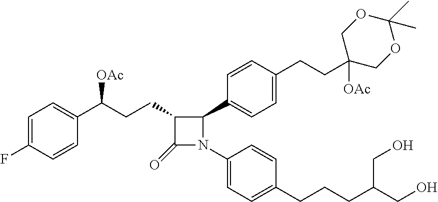

The title compound was prepared from (1S)-3-((2S,3R)-2-(4-{[5-(acetyloxy)-2,2-dimethyl-1,3-dioxan-5-yl]ethynyl}phenyl)-1-{4-[5-hydroxy-4-(hydroxymethyl)pent-1-yn-1-yl]phenyl}-4-oxoazetidin-3-yl)-1-(4-fluorophenyl)propyl acetate (from Step C) according to the procedure for Example 22, step B. m/z (ES) 774.4 (M-OAc)⁺.

### Step E: Preparation of (3R,4S)-4-{4-[3,4-dihydroxy-3-(hydroxymethyl)butyl]phenyl}-3-[(3S)-3-(4-fluorophenyl)-3-hydroxypropyl]-1-{4-[5-hydroxy-4-(hydroxymethyl)pentyl]phenyl}azetidin-2-one

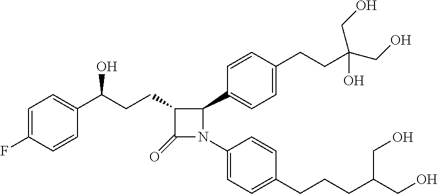

The title compound was prepared from (1S)-3-((2S,3R)-2-(4-{2-[5-(acetyloxy)-2,2-dimethyl-1,3-dioxan-5-yl]ethyl}phenyl)-1-{4-[5-hydroxy-4-(hydroxymethyl)pentyl]phenyl}-4-oxoazetidin-3-yl)-1-(4-fluorophenyl)propyl acetate (from Step D) according to the procedures for Example 1, Steps F and G. m/z (ES) 592.4 (M-OH)⁺, 532.4 (M+Na)⁺.

### Example 25

### (3R,4S)-1,4-bis{4-[3,4-dihydroxy-3-(hydroxymethyl)butyl]phenyl}-3-[(3S)-3-(4-fluorophenyl)-3-hydroxypropyl]azetidin-2-one

### Step A: Preparation of {(2S,3R)-3-[(3S)-3-(acetyloxy)-3-(4-fluorophenyl)propyl]-4-oxoazetidine-1,2-diyl}bis(4,1-phenyleneethyne-2,1-diyl-2,2-dimethyl-1,3-dioxane-5,5-diyl) diacetate

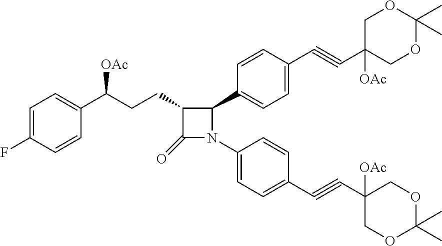

The title compound was prepared from (1S)-1-(4-fluorophenyl)-3-[(3R,4S)-1-(4-iodophenyl)-2-oxo-4-(4-{[(trifluoromethyl)sulfonyl]oxy}phenyl)azetidin-3-yl]propyl acetate (see Example 10, Step A) and 5-ethynyl-2,2-dimethyl-1,3-dioxan-5-yl acetate (i-6) according to the procedure for Example 1, step D. m/z (ES) 833.3 (M+Na)⁺, 750.4 (M-OAc)⁺.

### Step B: Preparation of 1(2S,3R)-3-[(3S)-3-(acetyloxy)-3-(4-fluorophenyl)propyl]-4-oxoazetidine-1,2-diyl}bis(4,1-phenyleneethane-2,1-diyl-2,2-dimethyl-1,3-dioxane-5,5-diyl)diacetate

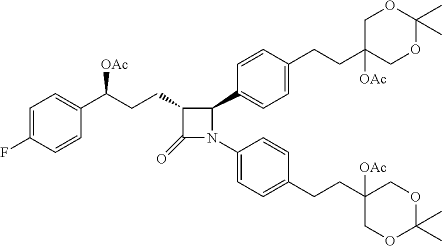

The bis-acetylene compound (310 mg) from Example 25, Step A was dissolved in ethanol (10 ml) and 20% palladium hydroxide on carbon (30 mg) was added to the solution. After three vacuum then flush with hydrogen cycles, the ethanol solution was hydrogenated at atmospheric pressure and at room temperature with hydrogen gas contained in a balloon. After 10 hours of hydrogenation, the reaction was judged to be essentially over by lc-ms. The spent hydrogenation catalyst was removed by filtering through a 0.45-micron Acrodisk syringe filter and the filtrates obtained concentrated down. The crude product was purified by preparative tlc on silica gel plates eluted with CH₂Cl₂ with MeOH (97:3 v/v) to afford the title compound. m/z (ES) 758.4 (M-OAc)⁺.

### Step C: Preparation of (3R,4S)-1,4-bis{-4-[3,4-dihydroxy-3-(hydroxymethyl)butyl]phenyl}-3-[(3S)-3-(4-fluorophenyl)-3-hydroxypropyl]azetidin-2-one

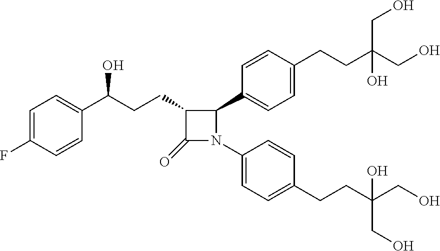

The title compound was prepared from ({(2S,3R)-3-[(3S)-3-(acetyloxy)-3-(4-fluorophenyl)propyl]-4-oxoazetidine-1,2-diyl}bis(4,1-phenyleneethane-2,1-diyl-2,2-dimethyl-1,3-dioxane-5,5-diyl)diacetate (Example 25, Step B) according to the procedures for Example 1, Steps F and G. ¹H-NMR (400 MHz, CD₃OD) δ: 1.67 (complex, 2H), 1.75 (complex, 2H), 1.93 (complex, 2H) 2.63 (t, J=7.5 Hz, 2H), 2.71 (t, J=7.5 Hz, 2H), 3.05 (broad m, 1H), 3.53 (s, 4H), 3.57 (s, 4H), 4.62 (broaden t, J=6 Hz, 1H), 4.80 (d, J=2 Hz, 1H), 7.00-7.37 (complex, 12H). m/z (ES) m/z=634.4 (M+Na)⁺; 771.8 (M-OH)⁺.

### Example 26

### N,N′-[2-[3-[4-[(2S,3R)-3-[(3S)-3-(4-fluorophenyl)-3-hydroxypropyl]-2-[4-[5-hydroxy-4-(hydroxymethyl)pentyl]phenyl]-4-oxo-1-azetidinyl]phenyl]propyl]-1,3-propanediyl]bis-methanesulfonamide

### Step A: Preparation of tert-Butyl (methylsulfonyl)carbamate

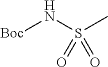

Methanesulfonamide (4.4 g; 46.26 mmol), triethylamine (7.1 ml; 50.88 mmol) and DMAP (565 mg; 4.626 mmol) were dissolved in dry CH₂Cl₂ (50 ml) and stirred at room temperature. A solution of di-tert-butyl dicarbonate (11.61 g; 53.196 mmol) in dry CH₂Cl₂ (100 ml) was slowly added drop by drop over 10 minutes. After the addition was complete the reaction mixture was stirred a further 1 hour, then the volatiles were removed under reduced pressure. The residues were carefully partitioned with 2N hydrochloric acid (150 ml) and diethyl ether (2×150 ml). The ether extracts were combined and washed with brine (150 ml) and the extract dried over anhydrous MgSO₄ powder, filtered and the filtrates concentrated under reduced pressure. The solid residues obtained from evaporation was triturated with hexanes, the hexane layer was filtered off and discarded. The remaining solid was crystallized from hexane and diethyl ether to give the title compound. ¹H-NMR (400 MHz, CDCl₃) δ: 1.53 (s, 9H), 3.28 (s., 3H).

### Step B: Preparation of N,N′-[2-(2-propynyl)-1,3-propanediyl]bis-methanesulfonamide

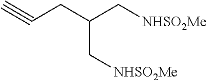

A solution comprising the diol, 2-prop-2-yn-1-ylpropane-1,3-diol (0.5 g; 4.381 mmol) from (Example 24, Step A), triphenylphosphine (2.53 g; 9.637 mmol) and tert-butyl (methylsulfonyl)carbamate (1.8 g; 9.199 mmol) in dry CH₂Cl₂ (30 ml) was stirred at 0° C. in an ice-water bath. Neat di-isopropylazodicarboxylate (2.16 ml; 10.951 mmol) was added drop by drop to the solution over 10 minutes and the reaction mixture stirred for 4 h during which time the ice-water bath had melted and had risen to room temperature. The progress of the reaction was checked by lc-ms (m/z=491.1 M+Na). The reaction solution was then evaporated under reduced pressure on a rotary evaporator. The crude product was purified by preparative tlc on silica gel plates eluted with EtOAc:Hex 1:4 v/v to give an oil. The product by NMR was still contaminated with di-isopropyl hydrazine-1,2-dicarboxylate. The oil was triturated with EtOAc:Hex (5:95 v/v) and crystallization induced by scratching. A copious white precipitate appeared. The solid was filtered off and the filtrates thus obtained (mixture of the desired bis-methanesulfonate and hydrazine-1,2-dicarboxylate (approx. 1:1) was concentrated under reduced pressure and the residues taken on to the deprotection step. The oil was dissolved in dry CH₂Cl₂ (5 ml) and anisole (1 ml) was added followed by trifluoroacetic acid (5 ml). The reaction mixture was stirred as a solution overnight at room temperature. After 18 hours, the volatiles were removed on a rotary evaporator under reduced pressure to leave an oil. The oil was partitioned with 1N aqueous sodium hydroxide solution (50 ml) and ether (2×50 ml). The ethereal extracts were combined and dried over anhydrous MgSO₄ powder, filtered and the filtrates concentrated under reduced pressure. The oil residue from evaporation was purified by preparative tlc on silica gel plates that were eluted with EtOAc:Hexanes (1:3 v/v) to afford the title compound. ¹H-NMR (400 MHz, CDCl₃) δ: 2.08 (complex., 2H), 2.30 (dd, J=2.5 Hz and 7 Hz 2H), 2.99 (s, 6H), 3.25 (m, 2H), 3.34 (m, 2H), 4.90 (broad t, 2H)

### Step C: (1S)-1-(4-fluorophenyl)-3-[(3R,4S)-1-[4-(5-[(methylsulfonyl)amino]-4-{[(methylsulfonyl)amino]methyl}pent-1-yn-1-yl)phenyl]-2-oxo-4-(4-{[(trifluoromethyl)sulfonyl]oxy}phenyl)azetidin-3-yl]propyl acetate

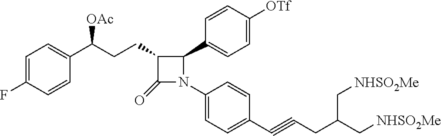

The title compound was prepared from the product of Step B and (1S)-1-(4-fluorophenyl)-3-[(3R,4S)-1-(4-iodophenyl)-2-oxo-4-(4-{[(trifluoromethyl)sulfonyl]-oxy}phenyl)azetidin-3-yl]propyl acetate (see Example 10, Step A) according to the procedure for Example 1, step A. m/z (ES) m/z=771.8 (M-OAc)⁺.

### Step D: (1S)-1-(4-fluorophenyl)-3-{(2S,3R)-2-{4-[5-hydroxy-4-(hydroxymethyl)pent-1-yn-1-yl]phenyl}-1-[4-(5-[(methylsulfonyl)amino]-4-{[(methylsulfonyl)amino]methyl}pent-1-yn-1-yl)phenyl]-4-oxoazetidin-3-yl}propyl acetate.

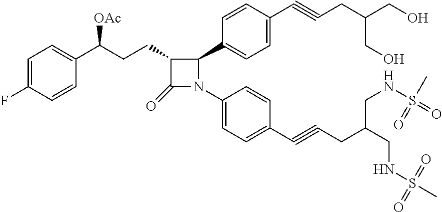

The title compound was prepared from the product from Step C and 2-prop-2-yn-1-ylpropane-1,3-diol (see Example 24, Step A) according to the procedure for Example 1, step D. m/z=736.1 (M-OAc)⁺.

### Step E: (1S)-1-(4-fluorophenyl)-3-{(2S,3R)-2-{4-[5-hydroxy-4-(hydroxymethyl)pentyl]phenyl}-1-[4-(5-[(methylsulfonyl)amino]-4-{[(methylsulfonyl)amino]methyl}pentyl)phenyl]-4-oxoazetidin-3-yl}propyl acetate

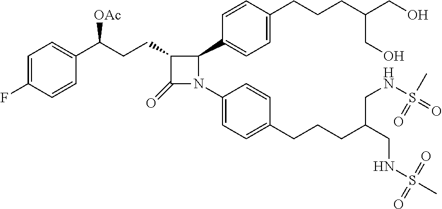

The title compound was prepared from the product of Step D) according to the procedure for Example 25, Step B. m/z (ES) 744.1 (M-OAc)⁺.

### Step F: N,N′-[2-[3-[4-[(2S,3R)-3-[(3S)-3-(4-fluorophenyl)-3-hydroxypropyl]-2-[4-[5-hydroxy-4-(hydroxymethyl)pentyl]phenyl]-4-oxo-1-azetidinyl]phenyl]propyl]-1,3-propanediyl]bis-methanesulfonamide

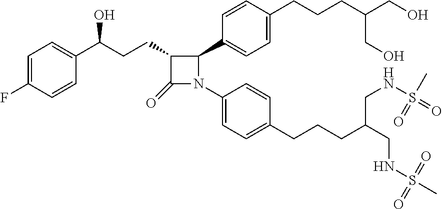

The title compound was prepared from the product of Step E (according to the procedure for Example 1, Step G. ¹H-NMR (500 MHz, CD₃OD) δ: 1.39 (complex, 4H), 1.65 (complex, 6H), 1.89 (complex, 4H), 2.53 (t, J=7.5 Hz, 2H), 2.60 (t, J=7.5 Hz, 2H), 2.88 (broad s, 6H), 3.02 (d, J=2 Hz, 4H), 3.53 (d, J=6 Hz, 4H), 4.59 (broad t, J=6 Hz, 1H), 4.77 (d, J=2 Hz, 1H), 6.90-7.32 (complex, 12H). m/z (ES) 744.2 (M-OAc)⁺.

Employing procedures similar to those described in Examples 26, the following compounds in Table 6 were prepared from their appropriate starting materials:

TABLE 6

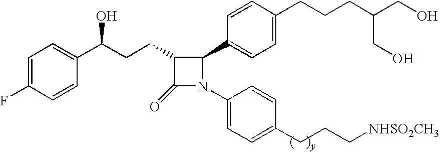

| Example | | |
|---|---|---|
| # | y | m/z (ES) |
| 27 | 3 | 595 (MH—H₂O)⁺ |
| 28 | 4 | 609 (MH—H₂O)⁺ |

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the mammal being treated for any of the indications for the active agents used in the instant invention as indicated above. Likewise, the specific pharmacological responses observed may vary according to and depending upon the particular active compound selected or whether there are present pharmaceutical carriers, as well as the type of formulation employed, and such expected variations or differences in the results are contemplated in accordance with the objects and practices of the present invention. It is intended, therefore, that the invention be defined by the scope of the claims which follow and that such claims be interpreted as broadly as is reasonable.

1. A compound of structural Formula Ia 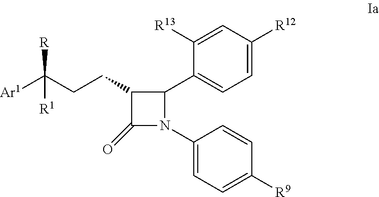
and the pharmaceutically acceptable salts thereof, wherein
Ar¹ is selected from the group consisting of aryl and R⁴-substituted aryl;
R is selected from the group consisting of —OR⁶, —O(CO)R⁶, —O(CO)OR⁸, —O(CO)NR⁶R⁷, a sugar residue, a disugar residue, a trisugar residue and a tetrasugar residue;
R¹ is selected from the group consisting of —H, —C₁₋₆alkyl and aryl;
R⁴ is 1-5 substituents independently selected at each occurrence from the group consisting of: —OR⁵, —O(CO)R⁵, —O(CO)OR⁸, —O—C₁₋₅alkyl-OR⁵, —O(CO)NR⁵R⁶, —NR⁵R⁶, —NR⁵(CO)R⁶, —NR⁵(CO)OR⁸, —NR⁵(CO)NR⁶R⁷, —NR⁵SO₂R⁸, —COOR⁵, —CONR⁵R⁶, —COR⁵, —SO₂NR⁵R⁶, —S(O)ₜR⁸, —O—C₁₋₁₀ alkyl-COOR⁵, —O—C₁₋₁₀alkyl-CONR⁵R⁶ and fluoro;
t is an integer selected from 0, 1 and 2;
R⁵, R⁶ and R⁷ are independently selected at each occurrence from the group consisting of —H, —C₁₋₆alkyl, aryl and aryl-substituted —C₁₋₆alkyl;
R⁸ is selected from the group consisting of —C₁₋₆alkyl, aryl and aryl-substituted —C₁₋₆alkyl;
R⁹ is selected from the group consisting of chloro, fluoro,
—C≡C—C₁₋₆alkyl-NR¹⁰R¹¹,
—(CH₂)ₓCH═CH—C₁₋₆alkyl-NR¹⁰R¹¹,
—C₁₋₈alkyl-NR¹⁰R¹¹,
—C≡C—C₁₋₄alkyl-CH—(CH₂—NR¹⁰R¹¹)₂,
—(CH₂)ₓCH═CH—C₁₋₄alkyl-CH—CH₂—NR¹⁰R¹¹)₂,
—C₁₋₆alkyl-CH—(CH₂—NR¹⁰R¹¹)₂,
—C≡C—C₁₋₆alkyl-R^{11a},
—(CH₂)ₓCH═CH—C₁₋₆alkyl-R^{11a},
—C₁₋₈ alkyl-R^{11a},
—C≡C—C₁₋₆alkyl,
—(CH₂)ₓCH═CH—C₁₋₆alkyl,
—C₁₋₈alkyl,
—C₂₋₁₅alkynyl mono- or poly-substituted with —OH and optionally substituted with R¹⁴,
—C₂₋₁₅alkenyl mono- or poly-substituted with —H and optionally substituted with R¹⁴,
—C₁₋₁₅alkyl mono- or poly-substituted with —OH and optionally substituted with R¹⁴,
and
x is an integer selected from 0, 1 and 2;
R¹⁰ is independently selected at each occurrence from the group consisting of —H and —C₁₋₃alkyl;
R¹¹ is independently selected at each occurrence from the group consisting of —H, —C₁₋₃alkyl, —C(O)—C₁₋₃alkyl, —C(O)—NR¹⁰R¹⁰, —SO₂—C₁₋₃alkyl and —SO₂-phenyl;
R^{11a} is selected from the group consisting of —C(O)—NR¹⁰R¹⁰, —SO₂—C₁₋₃alkyl, and —SO₂-phenyl;
R¹² is selected from the group consisting of —C₂₋₁₅alkynyl mono- or poly-substituted with —OH and optionally substituted with R¹⁴, —C₂₋₁₅alkenyl mono- or poly-substituted with —OH and optionally substituted with R¹⁴, —C₁₋₁₅alkyl mono- or poly-substituted with —OH and optionally substituted with R¹⁴;
R¹³ is selected from the group consisting of —H and —OH; and
R¹⁴ is a sugar residue optionally substituted with —COOH, —COOC₁₋₃alkyl and —C₁₋₃alkyl-OH;
provided that when R⁹ is selected from the group consisting of —C≡C—(CH₂)₁₋₆—NR¹⁰R¹¹, —CH═CH—(CH₂)₁₋₆—NR¹⁰R¹¹ and —(CH₂)₁₋₈—NR¹⁰R¹¹, then R¹² is not selected from the group consisting of —C₁₋₁₅alkyl mono- or poly-substituted with —OH, —CH═CH—C₁₋₁₃alkyl mono- or poly-substituted with —OH, —C≡C—C₁₋₁₃alkyl mono- or poly-substituted with —OH, and 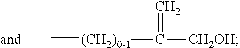
and excluding (3R,4S)-4-{4-[3,4-dihydroxy-3-(hydroxymethyl)butyl]phenyl}-3-[(3S)-3-(4-fluorophenyl)-3-hydroxypropyl]-1-[4-(3-hydroxypropyl)phenyl]azetidin-2-one.

2. The compound of claim 1 wherein Ar¹ is selected from the group consisting of aryl and R⁴-substituted aryl wherein R⁴ is 1-2 substituents independently selected at each occurrence from the group consisting of: —OR⁵, —O(CO)R⁵, —O(CO)OR⁸, —O—C₁₋₅alkyl-OR⁵, —O(CO)NR⁵R⁶, —NR⁵R⁶, —NR⁵(CO)R⁶, —NR⁵(CO)OR⁸, —NR⁵(CO)NR⁶R⁷, —NR⁵SO₂R⁸, —COOR⁵, —CONR⁵R⁶, —COR⁵, —SO₂NR⁵R⁶, —S(O)ₜR⁸, —O—C₁₋₁₀alkyl-COOR⁵, —O—C₁₋₁₀alkyl-CONR⁵R⁶ and fluoro.

3. The compound of claim 2 wherein R is —OR⁶ and R¹ is —H.

4. The compound of claim 1 having structural Formula Ib 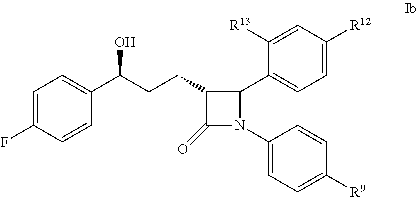
and the pharmaceutically acceptable salts thereof.

5. The compound of claim 4 selected from the group consisting of:
1) N-(5-[4-((2S,3R)-3-[(3S)-3-(4-fluorophenyl)-3-hydroxypropyl]-2-{4-[5-hydroxy-4-(hydroxymethyl)pentyl]phenyl}-4-oxoazetidin-1-yl)phenyl]-2-{[(methylsulfonyl)amino]methyl}pentyl)methanesulfonamide;
2) (3R,4S)-1,4-bis{4-[3,4-dihydroxy-3-(hydroxymethyl)butyl]phenyl}-3-[(3S)-3-(4-fluorophenyl)-3-hydroxypropyl]azetidin-2-one;
3) (3R,4S)-4-(4-[3,4-dihydroxy-3-(hydroxymethyl)butyl]phenyl}-3-[(3S)-3-(4-fluorophenyl)-3-hydroxypropyl]-1-{4-[5-hydroxy-4-(hydroxymethyl)pentyl]-phenyl}azetidin-2-one;
4) (3R,4S)-3-[(3S)-3-(4-fluorophenyl)-3-hydroxypropyl]-1,4-bis[4-(3-hydroxypropyl)phenyl]azetidin-2-one;
5) (3R,4S)-3-[(3S)−)-3-(4-fluorophenyl)-3-hydroxypropyl]-1,4-bis[4-(4-hydroxybutyl)phenyl]azetidin-2-one;
6) (3R,4S)-4-{4-[3,4-dihydroxy-3-(hydroxymethyl)butyl]phenyl}-1-[4-(2,3-dihydroxypropyl)phenyl]-3-[(3S—)-3-(4-fluorophenyl)-3-hydroxypropyl]azetidin-2-one;
7) (3R,4S)-1-[4-(1,2-dihydroxyethyl)phenyl]-4-{4-[3,4-dihydroxy-3-(hydroxymethyl)butyl]phenyl}-3-[(3S)-3-(4-fluorophenyl)-3-hydroxypropyl]azetidin-2-one
8) (3R,4S)-4-{4-[3,4-dihydroxy-3-(hydroxymethyl)butyl]phenyl}-3-[(3S)-3-(4-fluorophenyl)-3-hydroxypropyl]-1-(4-propylphenyl)azetidin-2-one;
9) (3R,4S)-4-(4-[3,4-dihydroxy-3-(hydroxymethyl)butyl]phenyl}-3-[(3S)-3-(4-fluorophenyl)-3-hydroxypropyl]-1-{4-[4-(methylsulfonyl)butyl]phenyl}azetidin-2-one;
10) (3R,4S)-4-{4-[3,4-dihydroxy-3-(hydroxymethyl)butyl]phenyl}-3-[(3S)-3-(4-fluorophenyl)-3-hydroxypropyl]1-{4-[6-(methylsulfonyl)hexyl]phenyl}azetidin-2-one;
11) methyl (2S,3S,4S,5R)-6-[4-{4-[(2S,3R)-3-[(3S)-3-(4-fluorophenyl)-3-hydroxypropyl]-1-(4-{3-[(methylsulfonyl)amino]propyl}phenyl)-4-oxoazetidin-2-yl]phenyl}-2-hydroxy-2-(hydroxymethyl)butoxy]-3,4,5-trihydroxytetrahydro-2H-pyran-2-carboxylate; and
12) (2S,3S,4,S,5R)-6-[4-{4-[(2S,3R)-3-[(3S)-3-(4-fluorophenyl)-3-hydroxypropyl]-1-(4-{3-[(methylsulfonyl)amino]propyl}phenyl)-4-oxoazetidin-2-yl]phenyl}-2-hydroxy-2-(hydroxymethyl)butoxy]-3,4,5-trihydroxytetrahydro-2H-pyran-2-carboxylic acid;
and the pharmaceutically acceptable salts thereof.

6. A method of reducing plasma LDL-cholesterol levels comprising administering a therapeutically effective amount of a compound of claim 1 to a patient in need of such treatment.

7. The method of claim 6 further comprising administering a therapeutically effective amount of a cholesterol biosynthesis inhibitor to a patient in need of such treatment.

8. A method of treating hypercholesterolemia comprising administering a therapeutically effective amount of a compound of claim 1 to a patient in need of such treatment.

9. A method of treating or reducing the risk for developing atherosclerosis comprising administering a therapeutically effective amount of a compound of claim 1 to a patient in need of such treatment.

10. A method of reducing the risk for having an atherosclerotic disease event comprising administering a prophylactically effective amount of a compound of claim 1 to a patient in at risk for such an event.

11. A pharmaceutical composition comprising the compound of claim 1 and a pharmaceutically acceptable carrier.

12. The pharmaceutical composition of claim 11 further comprising a therapeutically effective amount of a cholesterol biosynthesis inhibitor.

13. A compound selected from the group consisting of:
1) N-[4-(4-{(2S,3R)-2-{4-[3,4-dihydroxy-3-(hydroxymethyl)but-1-yn-1-yl]phenyl}-3-[(3S)-3-(4-fluorophenyl)-3-hydroxypropyl]-4-oxoazetidin-1-yl}phenyl)but-3-yn-1-yl]methanesulfonamide;
2) N-[5-(4-{(2S,3R)-2-{4-[3,4-dihydroxy-3-(hydroxymethyl)but-1-yn-1-yl]phenyl}-3-[(3S)-3-(4-fluorophenyl)-3-hydroxypropyl]-4-oxoazetidin-1-yl}phenyl)pent-4-yn-1-yl]methanesulfonamide;
3) N-[4-(4-{(2S,3R)-2-{4-[3,4-dihydroxy-3-(hydroxymethyl)butyl]phenyl}-3-[(3S)-3-(4-fluorophenyl)-3-hydroxypropyl]-4-oxoazetidin-1-yl}phenyl)butyl]methanesulfonamide;
4) N-[5-(4-{(2S,3R)-2-{4-[3,4-dihydroxy-3-(hydroxymethyl)butyl]phenyl}-3-[(3S)-3-(4-fluorophenyl)-3-hydroxypropyl]-4-oxoazetidin-1-yl}phenyl)pentyl]methanesulfonamide;
5) N-[6-(4-((2S,3R)-2-{4-[3,4-dihydroxy-3-(hydroxymethyl)butyl]phenyl}-3-[(3S)-3-(4-fluorophenyl)-3-hydroxypropyl]-4-oxoazetidin-1-yl}phenyl)hexyl]methanesulfonamide;
6) N-[4-(4-{(2S,3R)-3-[(3S)-3-(4-fluorophenyl)-3-hydroxypropyl]-2-[4-(7-hydroxyheptyl)phenyl]-4-oxoazetidin-1-yl}phenyl)butyl]methanesulfonamide;
7) N-[4-(4-((2S,3R)-3-[(3S)-3-(4-fluorophenyl)-3-hydroxypropyl]-2-{4-[5-hydroxy-4-(hydroxymethyl)pent-1-yn-1-yl]phenyl}-4-oxoazetidin-1-yl)phenyl]but-3-yn-1-yl}methanesulfonamide;
8) N-[5-(4-((2S,3R)-3-[(3S)-3-(4-fluorophenyl)-3-hydroxypropyl]-2-{4-[5-hydroxy-4-(hydroxymethyl)pent-1-yn-1-yl]phenyl}-4-oxoazetidin-1-yl)phenyl]pent-4-yn-1-yl}methanesulfonamide;
9) N-{6-[4-((2S,3R)-3-[(3S)-3-(4-fluorophenyl)-3-hydroxypropyl]-2-{4-[5-hydroxy-4-(hydroxymethyl)pent-1-yn-1-yl]phenyl}-4-oxoazetidin-1-yl)phenyl]hex-5-yn-1-yl}methanesulfonamide;
10) N-{5-[4-((2S,3R)-3-[(3S)-3-(4-fluorophenyl)-3-hydroxypropyl]-2-{4-[5-hydroxy-4-(hydroxymethyl)pentyl]phenyl}-4-oxoazetidin-1-yl)phenyl]pentyl)methanesulfonamide;
11) N-{6-[4-((2,S, 3R)-3-[(3S)-3-(4-fluorophenyl)-3-hydroxypropyl]-2-{4-[5-hydroxy-4-(hydroxymethyl)pentyl]phenyl)}-4-oxoazetidin-1-yl)phenyl]hexyl)methanesulfonamide;
12) N-[3-(4-{(2S,3R)-2-{4-[1,2-dihydroxy-1-(hydroxymethyl)ethyl]phenyl}-3-[(3S)-3-(4-fluorophenyl)-3-hydroxypropyl]-4-oxoazetidin-1-yl}phenyl)propyl]methanesulfonamide;
13) N-[3-(4-{(2S,3R)-2-{4-[4,5-dihydroxy-4-(hydroxymethyl)pentyl]phenyl}-3-[(3S)-3-(4-fluorophenyl)-3-hydroxypropyl]-4-oxoazetidin-1-yl}phenyl)propyl]methanesulfonamide;
14) N-[3-(4-{(2S,3R)-2-{4-[2,3-dihydroxy-2-(hydroxymethyl)propyl]phenyl}-3-[(3S)-3-(4-fluorophenyl)-3-hydroxypropyl]-4-oxoazetidin-1-yl}phenyl)propyl]methanesulfonamide;
15) N-[3-(4-{(2S,3R)-2-{4-[5,6-dihydroxy-5-(hydroxymethyl)hexyl]phenyl)-3-[(3S)-3-(4-fluorophenyl)-3-hydroxypropyl]-4-oxoazetidin-1-yl}phenyl)propyl]methanesuslfonamide; and
16) N-{3-[4-((3R,4S)-3-[(3S)-3-(4-fluorophenyl)-3-hydroxypropyl]-2-oxo-4-{4-[1,2,5,6-tetrahydroxy-5-(hydroxymethyl)hexyl]phenyl}azetidin-1-yl)phenyl]propyl}methanesulfonamide;
and the pharmaceutically acceptable salts thereof.

14. A pharmaceutical composition comprising the compound of claim 13 and a pharmaceutically acceptable carrier.

15. The pharmaceutical composition of claim 14 further comprising a therapeutically effective amount of a cholesterol biosynthesis inhibitor.

16. A method of reducing plasma LDL-cholesterol levels comprising administering a therapeutically effective amount of a compound of claim 13 to a patient in need of such treatment.

17. The method of claim 16 further comprising administering a therapeutically effective amount of a cholesterol biosynthesis inhibitor to a patient in need of such treatment.
